(19) United States
(12) Patent Application Publication
(10) Pub. No.: US 2010/0285149 A1
(43) Pub. Date: Nov. 11, 2010

(54) NOVEL TETRAHYDROPYRIDOTHIOPHENES

(76) Inventors: Klaus Pekari, Mittelbiberach (DE); Mathias Schmidt, Konstanz (DE); Astrid Zimmermann, Konstanz (DE); Volker Gekeler, Konstanz (DE); Thomas Beckers, Konstanz (DE); Thomas Bär, Reichenau (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/377,475

(22) PCT Filed: Aug. 15, 2007
(86) PCT No.: PCT/EP07/58432

§ 371 Date: Aug. 2, 2010

(30) Foreign Application Priority Data
Aug. 16, 2006 (EP) 06119034.4

(51) Int. Cl. A61K 33/24; A61K 31/5377; A61K 31/4365; C07D 413/14; C07D 495/04; A61P 35/02; A61P 35/00

(52) U.S. Cl. 424/649; 514/233.8; 514/301; 544/127; 546/114

(57) ABSTRACT

Compounds of a certain formula I, in which Ra and Rb have the meanings indicated in the description, are novel effective compounds with anti-proliferative and apoptosis inducing activity.

### FIELD OF APPLICATION OF THE INVENTION

The invention relates to tetrahydropyridothiophene derivatives, which can be used in the pharmaceutical industry for the production of pharmaceutical compositions.

The invention further relates to the contribution made to the art by the finding that said tetrahydro-pyridothiophene derivatives display cell-cycle dependent, anti-proliferative and apoptosis inducing activity.

The invention also relates to the use of these compounds for the therapy of hyperproliferative diseases, in particular human cancer.

### KNOWN TECHNICAL BACKGROUND

Cancer chemotherapy was established with the alkylating agent Cyclophosphamide (Endoxan®), an oxazaphosphorin pro-drug activated preferentially in the tumor. The target of alkylating agents like Cyclophosphamide is DNA and the concept, that cancer cells with uncontrolled proliferation and a high mitotic index are killed preferentially, proved to be very sucessfull. Standard cancer chemotherapeutic drugs finally kill cancer cells upon induction of programmed cell death (“apoptosis”) by targeting basic cellular processes and molecules. These basic cellular processes and molecules include RNA/DNA (alkylating and carbamylating agents, platin analogs and topoisomerase inhibitors), metabolism (drugs of this class are named anti-metabolites and examples are folic acid, purin and pyrimidine antagonist) as well as the mitotic spindle apparatus with αβ-tubulin heterodimers as the essential component (drugs are categorized into stabilizing and destabilizing tubulin inhibitors; examples are Taxol/Paclitaxel®, Docetaxel/Taxotere® and vinca alkaloids).

A subgroup of proapoptotic anticancer agents target cells preferentially in mitosis. In general these agents do not induce apoptosis in non-dividing cells, arrested in the G0, G1 or G2 phase of the cell division cycle. In contrast, dividing cells going through mitosis (M-phase of the cell division cycle), are killed efficiently by induction of apoptosis by this subgroup agents. Therefore, this subgroup or class of anti-cancer agents is described as cell-cycle specific or cell-cycle dependent. Tubulin inhibitors, with Taxol (Paclitaxel®) as a prominent example, belong to this class of cell-cycle specific, apoptosis inducing anti-cancer agents.

The international application WO2004024065 describes, inter alia, tetrahydropyridothiophene derivatives as glucagons antagonists for the treatment of diabetes.

The german document DE4039734 describes, inter alia, N-alkylated tetrahydropyridothiophene derivatives as components of herbicidal agents.

The german document DD272078 describes, inter alia, N-alkylated tetrahydropyridothiophene derivatives with antianaphylactic and antihistaminergic properties.

The international application WO2005033102 describes thiophene-based compounds exhibiting ATP-utilizing enzyme inhibitory activity.

The international application WO2004092156 describes substituted 3-cyanothiophene acetamides as glucagon receptor antagonists.

The international application WO9946267 describes 2-aminothiophene derivatives as modulators of protein tyrosine phosphatases.

The international application WO2005060711 describes a method of treating diseases mediated by sirtuin, e.g. SirT1 mediated deacetylation, using substituted thiophene compounds.

The international application WO2005033102 describes a method of combating phytopathogenic diseases on plants using 2-aminothiophene derivatives.

The international application WO2004069149 describes aminosulfonyl-substituted thienopyridine derivatives which are said to be capable of inhibiting the interactions between effector cell adhesion molecules and glycosaminoglycans and thus useful for treating diseases related to cell adhesion and cell migration.

The international applications WO2005118071, WO2005118592 and WO2005120642 describe tetra-hydropyridothiophenes with anti-proliferative and/or apoptosis inducing activity for use in the treatment of cancer.

### DESCRIPTION OF THE INVENTION

It has now been found that the tetrahydropyridothiophene derivatives, which are described in greater details below, differ from prior art compounds by unanticipated and originative structural alterations and have surprising and particularly advantageous properties.

Thus, for example, the compounds according to this invention are potent and highly efficacious inhibitors of cellular (hyper)proliferation and/or cell-cycle specific inducers of apoptosis in cancer cells.

Therefore, unanticipatedly, these compounds can be useful for treating (hyper)proliferative diseases and/or disorders responsive to the induction of apoptosis, in particular cancer. By having a cell-cycle specific mode of action, these derivates should have a higher therapeutic index compared to standard chemotherapeutic drugs targeting basic cellular molecules like DNA.

Thus, for example, the compounds according to this invention are expected to be useful in targeted cancer therapy.

The invention thus relates to compounds of formula I

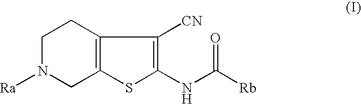

wherein
Ra is —C(O)—N(R11)-R1, in which
R1 is 1-4C-alkyl, 3-7C-cycloalkyl, HetA, phenyl, HarA, 1-4C-alkyl substituted by Raa, or 2-4C-alkyl substituted by Rab and Rac on different carbon atoms,
   wherein said 3-7C-cycloalkyl may be optionally substituted by one or two substituents independently selected from R12, and
   wherein each of said phenyl and HarA may be optionally substituted by one, two or three substituents independently selected from R13,
R11 is hydrogen, 1-4C-alkyl, 3-7C-cycloalkyl or 3-7C-cycloalkyl-1-4C-alkyl,
or R1 and R11 together and with inclusion of the nitrogen atom, to which they are attached, form a heterocyclic radical HET, in which
   either
HET is optionally substituted by one or two substituents independently selected from R12, and is piperidin-1-yl, pyrrolidin-1-yl, morpholin-4-yl, thiomorpholin-4-yl or 4N-(1-4C-alkylcarbonyl)-piperazin-1-yl,
   or
HET is optionally substituted by one or two substituents independently selected from R13, and is pyrrol-1-yl, imidazol-1-yl, pyrazol-1-yl or triazol-1-yl,
Rb is -T-Q, in which
T is a ethane-1,2-diyl, cyclopropane-1,2-diyl, or propane-1,2-diyl bridge, and
   either
Q is optionally substituted by Rba and/or Rbb, and is phenyl,
   or
Q is optionally substituted by Rca and/or Rcb, and is pyridyl,
   or
Q is optionally substituted by Rda and/or Rdb, and is furyl or thienyl,
   or
Q is optionally substituted by Rea and/or Reb, and is 3-7C-cycloalkyl,
   wherein
Raa is selected from the group consisting of:
   3-7C-cycloalkyl, phenyl,
   halogen, trifluoromethyl, cyano, hydroxyl,
   HarB, HetB, HetC, morpholino,
   —C(O)R2, —C(O)OR3, —C(O)N(R4)R5,
   —N(R4)R5, —N(R6)C(O)R7, —OC(O)R8,
   completely or predominantly fluorine-substituted 1-4C-alkoxy, and
   —OR9,
   wherein said 3-7C-cycloalkyl may be optionally substituted by one or two substituents independently selected from R12, and
   wherein each of said phenyl and HarB may be optionally substituted by one, two or three substituents independently selected from R13,
   in which
R2, R3, R4, R5, R6, R7 and R8 may be the same or different and are independently selected from the group consisting of:
   hydrogen and 1-4C-alkyl,
R9 is selected from the group consisting of:
   1-4C-alkyl, 3-7C-cycloalkyl, 3-7C-cycloalkyl-1-4C-alkyl, hydroxy-2-4C-alkyl, 1-4C-alkoxy-2-4C-alkyl, phenyl-1-4C-alkyl, pyridyl-1-4C-alkyl, and (1-4C-alkoxy-2-4C-alkoxy)-2-4C-alkyl,
      either
HarA is bonded to the parent molecular group via a ring carbon atom, and is a 5-membered monocyclic partially unsaturated or aromatic heterocyclic ring comprising one to four heteroatoms independently selected from nitrogen, oxygen and sulphur,
   or
HarA is bonded to the parent molecular group via a ring carbon atom, and is a 6-membered monocyclic partially unsaturated or aromatic heterocyclic ring comprising one or two nitrogen atoms,
   or
HarA is bonded to the parent molecular group via a ring carbon atom, and is a 5-membered mono-cyclic partially unsaturated or aromatic heterocyclic ring comprising one to three heteroatoms independently selected from nitrogen, oxygen and sulphur, which heterocyclic ring is substituted by one oxo group,
   or
HarA is bonded to the parent molecular group via a ring carbon atom, and is a 6-membered mono-cyclic partially unsaturated or aromatic heterocyclic ring comprising one or two nitrogen atoms, which heterocyclic ring is substituted by one oxo group,
   either
HarB is bonded to the parent molecular group via a ring carbon or a ring nitrogen atom, and is a 5-membered monocyclic partially unsaturated or aromatic heterocyclic ring comprising one to four heteroatoms independently selected from nitrogen, oxygen and sulphur,
   or
HarB is bonded to the parent molecular group via a ring carbon atom, and is a 6-membered mono-cyclic partially unsaturated or aromatic heterocyclic ring comprising one or two nitrogen atoms,
   or
HarB is bonded to the parent molecular group via a ring carbon or a ring nitrogen atom, and is a 5-membered monocyclic partially unsaturated or aromatic heterocyclic ring comprising one to three heteroatoms independently selected from nitrogen, oxygen and sulphur, which heterocyclic ring is substituted by one oxo group,
   or
HarB is bonded to the parent molecular group via a ring carbon or a ring nitrogen atom, and is a 6-membered monocyclic partially unsaturated or aromatic heterocyclic ring comprising one or two nitrogen atoms, which heterocyclic ring is substituted by one oxo group,
each R12 may be the same or different and is independently selected from the group consisting of:
   1-4C-alkyl, halogen, hydroxyl, and 1-4C-alkoxy,
each R13 may be the same or different and is independently selected from the group consisting of:
   1-4C-alkyl, halogen, hydroxyl, 1-4C-alkoxy, amino, amino-1-4C-alkyl, mono- or di-1-4C-alkyl-amino, hydroxy-2-4C-alkoxy, 1-4C-alkoxy-2-4C-alkoxy, hydroxy-1-4C-alkyl, and 1-4C-alkoxy-1-4C-alkyl,
HetA is bonded to the parent molecular group via a ring carbon atom, and is tetrahydropyranyl, tetrahydrofuryl, 1N-(1-4C-alkylcarbonyl)-piperidinyl, 1N-(1-4C-alkylcarbonyl)-pyrrolidinyl, 1N-(formyl)-piperidinyl, 1N-(formyl)-pyrrolidinyl, tetrahydrothiapyranyl, tetrahydrothienyl, 1N—(R14)-piperidin-2-onyl, 1N—(R14)-pyrrolidin-2-onyl, tetrahydropyran-2-onyl, tetrahydrofuran-2-onyl, 3N—(R14)-oxazolidin-2-onyl, or 1N—(R14)-3N—(R15)-imidazolidin-2-onyl, wherein each of said HetA may be optionally substituted by one or two substituents independently selected from R16,
HetB is bonded to the parent molecular group via a ring nitrogen atom, and is piperidin-2-on-1-yl, pyrrolidin-2-on-1-yl, oxazolidin-2-on-1-yl, or 3N—(R15)-imidazolidin-2-on-1-yl, wherein each of said HetB may be optionally substituted by one or two substituents independently selected from R16,
HetC is bonded to the parent molecular group via a ring carbon atom, and is tetrahydropyranyl, tetrahydrofuryl, 1N-(1-4C-alkylcarbonyl)-piperidinyl, 1N-(1-4C-alkylcarbonyl)-pyrrolidinyl, 1N-(formyl)-piperidinyl, 1N-(formyl)-pyrrolidinyl, tetrahydrothiapyranyl, tetrahydrothienyl, 1N—(R14)-piperidin-2-onyl, 1N—(R14)-pyrrolidin-2-onyl, tetrahydropyran-2-onyl, tetrahydrofuran-2-onyl, 3N—(R14)-oxazolidin-2-onyl, or 1N—(R14)-3N—(R15)-imidazolidin-2-onyl,
   wherein each of said HetC may be optionally substituted by one or two substituents independently selected from R16, in which
R14 is hydrogen or 1-4C-alkyl,
R15 is hydrogen or 1-4C-alkyl,
each R16 may be the same or different and is independently selected from the group consisting of:
   1-4C-alkyl, halogen, hydroxyl, and 1-4C-alkoxy,
Rab is hydroxyl,
Rac is hydroxyl,
or Rab and Rac bonded to adjacent carbon atoms form together an 1-2C-alkylenedioxy bridge which is optionally substituted by one or two substituents independently selected from fluorine and methyl,
or Rab and Rac bonded to carbon atoms two bonds distant from each other form together a methylenedioxy bridge which is optionally substituted by one or two substituents independently selected from fluorine and methyl,
Rba is 1-4C-alkyl, 1-4C-alkoxy or halogen,
Rbb is 1-4C-alkyl, 1-4C-alkoxy or halogen,
Rca is 1-4C-alkyl, 1-4C-alkoxy or halogen,
Rcb is 1-4C-alkyl, 1-4C-alkoxy or halogen,
Rda is 1-4C-alkyl or halogen,
Rdb is 1-4C-alkyl or halogen,
Rea is 1-4C-alkyl, 1-4C-alkoxy, halogen or hydroxyl,
Reb is 1-4C-alkyl, 1-4C-alkoxy, halogen or hydroxyl,
   and the salts, as well as the stereoisomers and salts of the stereoisomers thereof.

As used herein, “alkyl” alone or as part of another group refers to both branched and straight chain saturated aliphatic hydrocarbon groups having the specified numbers of carbon atoms, such as for example:

1-4C-Alkyl is a straight-chain or branched alkyl radical having 1 to 4 carbon atoms. Examples are the butyl, isobutyl, sec-butyl, tert-butyl, propyl, isopropyl, ethyl and methyl radicals, of which propyl, isopropyl, ethyl and methyl are more worthy to be mentioned.
2-4C-Alkyl is a straight-chain or branched alkyl radical having 2 to 4 carbon atoms. Examples are the butyl, isobutyl, sec-butyl, tert-butyl, propyl, isopropyl and ethyl radicals, of which propyl, isopropyl and ethyl are more worthy to be mentioned.

Ethane-1,2-diyl stands for the ethylene (—CH₂—CH₂—) radical.

Cyclopropane-1,2-diyl stands for the 1,2-cyclopropylene radical, preferably the trans isomer thereof.

Propane-1,2-diyl stands for the 1,2-propylene (2-methylethylene) radical [—CH₂—CH(CH₃)—] including (R)-1,2-propylene and (S)-1,2-propylene, whereby it is to be understood, that, when T is from formula —CH₂—CH(CH₃)—, said radical is attached with its right terminus to the moiety Q.

1-4C-alkyl substituted by Raa stands for one of the abovementioned 1-4C-alkyl radicals which is substituted by a Raa radical as defined herein, such as e.g. (Raa)-methyl [(Raa)-CH₂—], 2-(Raa)-ethyl [(Raa)-CH₂—CH₂—], 3-(Raa)-propyl [(Raa)-CH₂—CH₂—CH₂—], or 1-(Raa)-ethyl [(Raa)-C(CH₃)H—] including (S)-1-(Raa)-ethyl and (R)-1-(Raa)-ethyl.

The term “cycloalkyl” alone or as part of another group refers to a monocyclic saturated aliphatic hydrocarbon group having the specified numbers of ring carbon atoms, such as for example:

3-7C-Cycloalkyl stands for cyclopropyl, cyclobutyl, cyclopentyl, cyclohexyl and cycloheptyl.
3-7C-Cycloalkyl-1-4C-alkyl stands for one of the abovementioned 1-4C-alkyl radicals, which is substituted by one of the abovementioned 3-7C-cycloalkyl radicals. Examples which may be mentioned are the 2-(3-7C-cycloalkyl)ethyl and, particularly, 3-7C-cycloalkylmethyl radicals, e.g. the 2-cyclohexylethyl or the cyclopropylmethyl, cyclobutylmethyl or cyclopentylmethyl radical, particularly the cyclo-propylmethyl radical.

Phenyl-1-4C-alkyl represents one of the abovementioned 1-4C-alkyl radicals, which is substituted by a phenyl radical. Examples which may be mentioned are the phenethyl and the benzyl radicals.

Pyridyl-1-4C-alkyl represents one of the abovementioned 1-4C-alkyl radicals, which is substituted by a pyridyl radical. Examples which may be mentioned are the 2-pyridyl-ethyl and the pyridylmethyl radicals.

Pyridyl includes pyridin-2-yl, pyridin-3-yl and pyridin-4-yl.

Halogen within the meaning of the present invention is iodine, or, particularly, bromine, chlorine and fluorine.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy, ethoxy and methoxy radicals, of which propoxy, isopropoxy, and, particularly, ethoxy and methoxy are more worthy to be mentioned.

2-4C-Alkoxy represents radicals which, in addition to the oxygen atom, contain a straight-chain or branched alkyl radical having 2 to 4 carbon atoms. Examples which may be mentioned are the butoxy, isobutoxy, sec-butoxy, tert-butoxy, propoxy, isopropoxy and ethoxy radicals, of which propoxy, isopropoxy, and, particularly, ethoxy are more worthy to be mentioned.

1-2C-Alkylenedioxy represents, for example, the methylenedioxy[-O—CH₂—O—] and the ethylenedioxy [—O—CH₂—CH₂—O—] radicals.

An 1-2C-alkylenedioxy bridge which is optionally substituted by one or two substituents independently selected from fluorine and methyl refers, for example, to the methylenedioxy[-O—CH₂—O—], the ethylenedioxy [—O—CH₂—CH₂—O—], the dimethylmethylenedioxy[-O—C(CH₃)₂—O—] or the difluoromethylenedioxy [—O—CF₂—O—] radicals.

A methylenedioxy bridge which is optionally substituted by one or two substituents independently selected from fluorine and methyl refers, for example, to the methylenedioxy[-O—CH₂—O—], the dimethylmethylenedioxy[-O—C(CH₃)₂—O—] or the difluoromethylenedioxy[-O—CF₂—O—] radicals.

As completely or predominantly fluorine-substituted 1-4C-alkoxy, for example, the 2,2,3,3,3-pentafluoro-propoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals may be mentioned. “Predominantly” in this connection means that more than half of the hydrogen atoms of the 1-4C-alkoxy radicals are replaced by fluorine atoms.

1-4C-Alkoxy-1-4C-alkyl represents one of the abovementioned 1-4C-alkyl radicals, which is substituted by one of the abovementioned 1-4C-alkoxy radicals. Examples which may be mentioned are the methoxymethyl, ethoxymethyl, isopropoxymethyl, 2-methoxyethyl, 2-ethoxyethyl and the 2-isopro-poxyethyl radicals.

1-4C-Alkoxy-2-4C-alkyl represents one of the abovementioned 2-4C-alkyl radicals, which is substituted by one of the abovementioned 1-4C-alkoxy radicals. Examples which may be mentioned are the 2-methoxyethyl, 2-ethoxyethyl and the 2-isopropoxyethyl radicals.

1-4C-alkoxy-2-4C-alkoxy represents one of the abovementioned 2-4C-alkoxy radicals, which is substituted one of the abovementioned 1-4C-alkoxy radicals. Examples which may be mentioned are the 2-methoxyethoxy, 2-ethoxyethoxy and the 2-isopropoxyethoxy radicals.

(1-4C-Alkoxy-2-4C-alkoxy)-2-4C-alkyl represents 2-4C-alkyl radicals, which are substituted by one of the abovementioned 1-4C-alkoxy-2-4C-alkoxy radicals. Examples which may be mentioned are the 2-(2-methoxyethoxy)-ethyl and the 2-(2-ethoxyethoxy)-ethyl radicals.

Hydroxy-1-4C-alkyl represents one of the abovementioned 1-4C-alkyl radicals, which is substituted by a hydroxyl group. Examples which may be mentioned are the hydroxymethyl, 2-hydroxyethyl and the 3-hydroxypropyl radicals, of which the hydroxymethyl radical is more worthy to be mentioned.

Hydroxy-2-4C-alkyl represents one of the abovementioned 2-4C-alkyl radicals, which is substituted by a hydroxyl group. Examples which may be mentioned are the 2-hydroxyethyl and the 3-hydroxypropyl radicals.

Hydroxy-2-4C-alkoxy represents one of the abovementioned 2-4C-alkoxy radicals, which is substituted by a hydroxyl group. Examples which may be mentioned are the 2-hydroxyethoxy and the 3-hydroxy-propoxy radicals.

1-4C-Alkylcarbonyl is a carbonyl group to which one of the abovementioned 1-4C-alkyl radicals is bonded. An example is the acetyl radical (CH₃CO—).

In addition to the nitrogen atom, mono- or di-1-4C-alkylamino radicals contain one or two of the abovementioned 1-4C-alkyl radicals. Mono-1-4C-alkylamino is to be mentioned and here, in particular, methyl-, ethyl- or isopropylamino. Di-1-4C-alkylamino is also to be mentioned and here, in particular, dimethyl-, diethyl- or diisopropylamino.

Amino-1-4C-alkyl stands for one of the abovementioned 1-4C-alkyl radicals, particularly 1-2C-alkyl, which is substituted by the amino radical. Examples, which may be mentioned, are the 2-aminoethyl and the aminomethyl radical.

4N-(1-4C-alkylcarbonyl)-piperazin-1-yl refers to the piperazin-1-yl radical, which is substituted on the nitrogen in 4-position by one of the aforementioned 1-4C-alkylcarbonyl radicals, such as e.g. 4-acetyl-piperazin-1-yl.

1N-(1-4C-alkylcarbonyl)-piperidinyl or 1N-(formyl)-piperidinyl, or 1N-(1-4C-alkylcarbonyl)-pyrrolidinyl or 1N-(formyl)-pyrrolidinyl refers to the piperidinyl or pyrrolidinyl radical, respectively, each of which is substituted on the nitrogen in 1-position by one of the aforementioned 1-4C-alkylcarbonyl radicals or formyl, respectively, such as e.g. 1N-(acetyl)-piperidinyl (e.g. 1-acetyl-piperidin-2-yl, 1-acetyl-piperidin-3-yl or 1-acetyl-piperidin-4-yl) or 1-formyl-piperidin-2-yl, 1-formyl-piperidin-3-yl or 1-formyl-piperidin-4-yl, or 1N-(acetyl)-pyrrolidinyl (e.g. 1-acetyl-pyrrolidin-2-yl or 1-acetyl-pyrrolidin-3-yl) or 1-formyl-pyrrolidin-2-yl or 1-formyl-pyrrolidin-3-yl.

1N—(R14)-piperidin-2-ony refers to any of the following radicals:

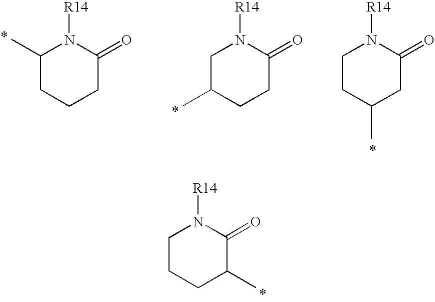

1N—(R14)-pyrrolidin-2-onyl refers to any of the following radicals:

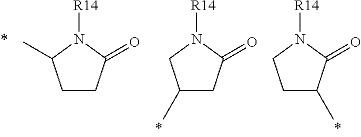

1N—(R14)-3N—(R15)-imidazolidin-2-onyl refers to any of the following radicals:

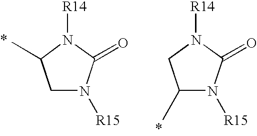

3N—(R14)-oxazolidin-2-onyl refers to any of the following radicals:

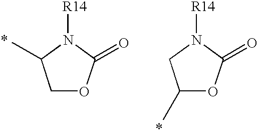

Tetrahydropyran-2-onyl refers to any of the following radicals:

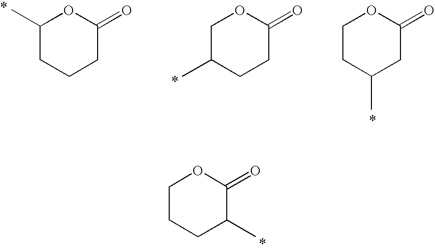

Tetrahydrofuran-2-onyl refers to any of the following radicals:

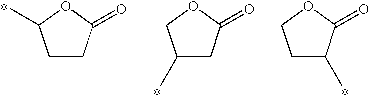

The asterisk in any of the above formulae marks the bond which connects the respective radical to the remainder of the molecule.

The term “radical” as used herein has the same meaning as the term “moiety” or “substituent”. Thus, the term “radical” is used in a formalistic way and relates to atoms and/or atom groups which can be attached at certain positions to the remainder of a molecule.

In a first embodiment HarA is bonded to the parent molecular group via a ring carbon atom, and is a 5-membered monocyclic partially unsaturated or aromatic heterocyclic ring comprising one, two, three or four heteroatoms independently selected from nitrogen, oxygen and sulphur.

Examples for HarA according to this first embodiment may include, but are not limited to, the heteroaryl derivatives thereof such as furanyl, thiophenyl, pyrrolyl, oxazolyl, isoxazolyl, thiazolyl, isothiazolyl, imidazolyl, pyrazolyl, triazolyl (including 1,2,3-triazolyl, 1,2,4-triazolyl and 1,3,4-triazolyl), thiadiazolyl (including 1,2,3-thiadiazolyl, 1,2,4-thiadiazolyl, 1,2,5-thiadiazolyl and 1,3,4-thiadiazolyl) or oxadiazolyl (including 1,2,3-oxadiazolyl, 1,2,4-oxadiazolyl, 1,2,5-oxadiazolyl and 1,3,4-oxadiazolyl). Further examples for HarA according to this first embodiment may include, but are not limited to, the partially unsaturated derivatives thereof such as 4,5-dihydro-oxazolyl (e.g. 4,5-dihydro-oxazol-2-yl or 4,5-dihydro-oxazol-4-yl) or 4,5-dihydro-thiazolyl (e.g. 4,5-dihydro-thiazol-2-yl or 4,5-dihydro-thiazol-4-yl).

In a second embodiment HarA is bonded to the parent molecular group via a ring carbon atom, and is a 6-membered monocyclic partially unsaturated or aromatic heterocyclic ring comprising one or two nitrogen atoms.

Examples for HarA according to this second embodiment may include, but are not limited to, the heteroaryl derivatives thereof such as pyridyl, pyrimidyl, pyrazinyl or pyridazinyl.

In a third embodiment HarA is bonded to the parent molecular group via a ring carbon atom, and is a 5-membered monocyclic partially unsaturated or aromatic heterocyclic ring comprising one, two or three heteroatoms independently selected from nitrogen, oxygen and sulphur, which heterocyclic ring is substituted by one oxo group.

Examples for HarA according to this third embodiment may include, but are not limited to, oxo-substituted derivatives of the above-mentioned examples of the first embodiment of HarA, such as e.g. oxazol-2-onyl, thiazol-2-onyl, imidazol-2-onyl, 1,3,4-oxadiazol-2-onyl, 1,2,4-oxadiazol-5-onyl, 1,2,4-oxadiazol-3-onyl, 1,3,4-triazol-2-onyl, 1,2,4-triazol-3-onyl, 1,2,4-triazol-5-onyl, 1,3,4-thiadiazol-2-onyl, 1,2,4-thiadiazol-5-onyl or 1,2,4-thiadiazol-3-onyl; or 4,5-dihydro-oxazol-5-onyl (e.g. 5-oxo-4,5-dihydro-5-oxo-oxazol-2-yl) or 4,5-dihydro-thiazol-5-onyl (e.g. 5-oxo-4,5-dihydro-thiazol-2-yl).

In a fourth embodiment HarA is bonded to the parent molecular group via a ring carbon atom, and is a 6-membered monocyclic partially unsaturated or aromatic heterocyclic ring comprising one or two nitrogen, which heterocyclic ring is substituted by one oxo group.

Examples for HarA according to this fourth embodiment may include, but are not limited to, oxo-substituted derivatives of the above-mentioned examples of the second embodiment of HarA, such as e.g. pyridin-2-onyl (2-pyridonyl), pyridin-4-onyl (4-pyridonyl), pyridazin-3-onyl, or pyrimidin-2-onyl.

In a first embodiment HarB is bonded to the parent molecular group via a ring carbon or a ring nitrogen atom, and is a 5-membered monocyclic partially unsaturated or aromatic heterocyclic ring comprising one, two, three or four heteroatoms independently selected from nitrogen, oxygen and sulphur.

Examples for HarB according to this first embodiment may include, but are not limited to, the heteroaryl derivatives thereof such as furanyl, thiophenyl, pyrrolyl, oxazolyl, isoxazolyl, thiazolyl, isothiazolyl, imidazolyl, pyrazolyl, triazolyl (including 1,2,3-triazolyl, 1,2,4-triazolyl and 1,3,4-triazolyl), thiadiazolyl (including 1,2,3-thiadiazolyl, 1,2,4-thiadiazolyl, 1,2,5-thiadiazolyl and 1,3,4-thiadiazolyl) or oxadiazolyl (including 1,2,3-oxadiazolyl, 1,2,4-oxadiazolyl, 1,2,5-oxadiazolyl and 1,3,4-oxadiazolyl), from which oxazolyl, thiazolyl, isoxazolyl, isothiazolyl, imidazolyl, pyrazolyl, oxadiazolyl (e.g. 1,3,4-oxadiazolyl), thiadiazolyl or triazolyl (e.g. 1,2,4-triazolyl) are more worthy to be mentioned.

Further examples for HarB according to this first embodiment may include, but are not limited to, the partially unsaturated derivatives thereof such as 4,5-dihydro-oxazolyl (e.g. 4,5-dihydro-oxazol-2-yl or 4,5-dihydro-oxazol-4-yl) or 4,5-dihydro-thiazolyl (e.g. 4,5-dihydro-thiazol-2-yl or 4,5-dihydro-thiazol-4-yl).

A more detailed example for HarB according to this first embodiment includes imidazolyl.

A further more detailed example for HarB according to this first embodiment includes imidazol-1-yl.

Another further more detailed example for HarB according to this first embodiment includes 1H-imidazolyl, e.g. imidazol-4-yl, imidazol-5-yl and imidazol-2-yl.

Another more detailed example for HarB according to this first embodiment includes isoxazolyl.

A further more detailed example for HarB according to this first embodiment includes isoxazol-3-yl.

Another further more detailed example for HarB according to this first embodiment includes isoxazol-4-yl.

Another further more detailed example for HarB according to this first embodiment includes isoxazol-5-yl.

Another more detailed example for HarB according to this first embodiment includes isothiazolyl.

A further more detailed example for HarB according to this first embodiment includes isothiazol-3-yl.

Another further more detailed example for HarB according to this first embodiment includes isothiazol-4-yl.

Another further more detailed example for HarB according to this first embodiment includes isothiazol-5-yl.

Another more detailed example for HarB according to this first embodiment includes thiazolyl.

A further more detailed example for HarB according to this first embodiment includes thiazol-2-yl.

Another further more detailed example for HarB according to this first embodiment includes thiazol-4-yl.

Another more detailed example for HarB according to this first embodiment includes oxazolyl.

A further more detailed example for HarB according to this first embodiment includes oxazol-2-yl.

Another further more detailed example for HarB according to this first embodiment includes oxazol-4-yl.

Another more detailed example for HarB according to this first embodiment includes oxadiazolyl, e.g. 1,3,4-oxadiazolyl.

A further more detailed example for HarB according to this first embodiment includes 1,3,4-oxadiazol-2-yl.

Another more detailed example for HarB according to this first embodiment includes triazolyl, e.g. 1,2,4-triazolyl.

A further more detailed example for HarB according to this first embodiment includes triazol-1-yl.

Another further more detailed example for HarB according to this first embodiment includes 1H-triazolyl, e.g. 1,2,4-triazol-5-yl.

Another more detailed example for HarB according to this first embodiment includes pyrazolyl.

A further more detailed example for HarB according to this first embodiment includes pyrazol-1-yl.

Another further more detailed example for HarB according to this first embodiment includes 1H-pyrazolyl, e.g. pyrazol-4-yl and pyrazol-5-yl.

Another more detailed example for HarB according to this first embodiment includes 4,5-dihydro-oxazolyl.

A further more detailed example for HarB according to this first embodiment includes 4,5-dihydro-oxazol-2-yl or 4,5-dihydro-oxazol-4-yl.

In a second embodiment HarB is bonded to the parent molecular group via a ring carbon atom, and is a 6-membered monocyclic partially unsaturated or aromatic heterocyclic ring comprising one or two nitrogen atoms.

Examples for HarB according to this second embodiment may include, but are not limited to, the heteroaryl derivatives thereof such as pyridyl, pyrimidyl, pyrazinyl or pyridazinyl.

A more detailed example for HarB according to this second embodiment includes pyridyl.

A further more detailed example for HarB according to this second embodiment includes pyridin-2-yl.

Another further more detailed example for HarB according to this second embodiment includes pyridin-3-yl.

Another further more detailed example for HarB according to this second embodiment includes pyridin-4-yl.

In a third embodiment HarB is bonded to the parent molecular group via a ring carbon or ring nitrogen atom, and is a 5-membered monocyclic partially unsaturated or aromatic heterocyclic ring comprising one, two or three heteroatoms independently selected from nitrogen, oxygen and sulphur, which heterocyclic ring is substituted by one oxo group.

Examples for HarB according to this third embodiment may include, but are not limited to, oxo-substituted derivatives of the above-mentioned examples of the first embodiment of HarB, such as e.g. oxazol-2-onyl, thiazol-2-onyl, imidazol-2-onyl, 1,3,4-oxadiazol-2-onyl, 1,2,4-oxadiazol-5-onyl, 1,2,4-oxadiazol-3-onyl, 1,3,4-triazol-2-onyl, 1,2,4-triazol-3-onyl, 1,2,4-triazol-5-onyl, 1,3,4-thiadiazol-2-onyl, 1,2,4-thiadiazol-5-onyl or 1,2,4-thiadiazol-3-onyl; or 4,5-dihydro-oxazol-5-onyl (e.g. 5-oxo-4,5-dihydro-5-oxo-oxazol-2-yl) or 4,5-dihydro-thiazol-5-onyl (e.g. 5-oxo-4,5-dihydro-thiazol-2-yl).

In a fourth embodiment HarB is bonded to the parent molecular group via a ring carbon or ring nitrogen atom, and is a 6-membered monocyclic partially unsaturated or aromatic heterocyclic ring comprising one or two nitrogen, which heterocyclic ring is substituted by one oxo group.

Examples for HarB according to this fourth embodiment may include, but are not limited to, oxo-substituted derivatives of the above-mentioned examples of the second embodiment of HarB, such as e.g. pyridin-2-onyl (2-pyridonyl), pyridin-4-onyl (4-pyridonyl), pyridazin-3-onyl, or pyrimidin-2-onyl.

The following expressions illustrate the moiety HarA or HarB, each of which is substituted by one, two or three substituents independently selected from R13:

Mono- or di-(1-4C-alkyl)-substituted imidazol-1-yl, pyrazol-1-yl or triazol-1-yl, respectively, stands for an imidazol-1-yl, pyrazol-1-yl or triazol-1-yl radical, respectively, which is substituted independently by one or two 1-4C-alkyl radicals as given above, such as mono- or di-methyl-substituted imidazol-1-yl, pyrazol-1-yl or triazol-1-yl, respectively, like 2-methyl-imidazol-1-yl, 4-methyl-imidazol-1-yl or 5-methyl-imidazol-1-yl, or 2,4-dimethyl-imidazol-1-yl; in particular 4-methyl-imidazol-1-yl.
Mono- or di-(1-4C-alkyl)-substituted isoxazolyl, oxazolyl, thiazolyl, thiadiazolyl, oxadiazolyl, isothiazolyl, 4,5-dihydro-oxazolyl or 4,5-dihydro-thiazolyl, respectively, stands for an isoxazolyl, oxazolyl, thiazolyl, thiadiazolyl, oxadiazolyl, isothiazolyl, 4,5-dihydro-oxazolyl or 4,5-dihydro-thiazolyl radical, respectively, which is substituted independently by one or two 1-4C-alkyl radicals as given above, such as mono- or di-methyl-substituted isoxazolyl, oxazolyl, thiazolyl, thiadiazolyl, oxadiazolyl, isothiazolyl, 4,5-dihydro-oxazolyl or 4,5-dihydro-thiazolyl, respectively, like methyl-substituted isoxazol-3-yl, methyl-substituted isoxazol-4-yl or methyl-substituted isoxazol-5-yl (e.g. 3-methyl-isoxazol-4-yl, 5-methyl-isoxazol-4-yl, 3-methyl-isoxazol-5-yl or 5-methyl-isoxazol-3-yl), methyl-substituted thiazol-4-yl or methyl-substituted thiazol-2-yl (e.g. 2-methyl-thiazol-4-yl or 4-methyl-thiazol-2-yl), methyl-substituted oxazol-4-yl or methyl-substituted oxazol-2-yl (e.g. 2-methyl-oxazol-4-yl or 4-methyl-oxazol-2-yl), methyl-substituted thiadiazolyl, e.g. methyl-substituted 1,3,4-thiadiazol-2-yl (e.g. 5-methyl-1,3,4-thiadiazol-2-yl), methyl-substituted oxadiazolyl, e.g. methyl-substituted 1,3,4-oxadiazol-2-yl (e.g. 5-methyl-1,3,4-oxadiazol-2-yl), methyl-substituted isothiazol-3-yl, methyl-substituted isothiazol-4-yl or methyl-substituted isothiazol-5-yl (e.g. 3-methyl-isothiazol-4-yl, 5-methyl-isothiazol-4-yl, 3-methyl-isothiazol-5-yl or 5-methyl-isothiazol-3-yl), methyl-substituted 4,5-dihydro-oxazol-2-yl or methyl-substituted 4,5-dihydro-oxazol-4-yl (e.g. 4-methyl-4,5-dihydro-oxazol-2-yl or 2-methyl-4,5-dihydro-oxazol-4-yl), or methyl-substituted 4,5-dihydro-thiazol-2-yl or methyl-substituted 4,5-dihydro-thiazol-4-yl (e.g. 4-methyl-4,5-dihydro-thiazol-2-yl or 2-methyl-4,5-dihydro-thiazol-4-yl).
1N-(1-4C-alkyl)-imidazolyl, 1N-(1-4C-alkyl)-pyrazolyl, 1N-(1-4C-alkyl)-triazolyl or 1N-(1-4C-alkyl)-pyrrolyl refers to imidazolyl, pyrazolyl, triazolyl or pyrrolyl, respectively, which is substituted by 1-4C-alkyl on the nitrogen atom in position 1, such as e.g. 1N-methyl-imidazolyl, 1N-ethyl-imidazolyl, 1N-methyl-pyrazolyl, 1N-ethyl-pyrazolyl, 1N-methyl-triazolyl, 1N-ethyl-triazolyl, 1N-methyl-pyrrolyl or 1N-ethyl-pyrrolyl, e.g. 1-methyl-imidazol-2-yl, 1-methyl-imidazol-5-yl, 1-ethyl-imidazol-2-yl, 1-methyl-imidazol-4-yl, 1-methyl-pyrazol-4-yl, 1-methyl-pyrazol-5-yl, 1-ethyl-pyrazol-5-yl, 1-methyl-1,2,4-triazol-5-yl, 1-ethyl-1,2,4-triazol-5-yl, 1-methyl-pyrrol-2-yl or 1-ethyl-pyrrol-2-yl.

1-4C-alkyl-substituted 1N-(1-4C-alkyl)-imidazolyl, 1-4C-alkyl-substituted 1N-(1-4C-alkyl)-pyrazolyl, 1-4C-alkyl-substituted 1N-(1-4C-alkyl)-triazolyl or 1-4C-alkyl-substituted 1N-(1-4C-alkyl)-pyrrolyl may include, for example, 1N-(1-4C-alkyl)-imidazolyl, 1N-(1-4C-alkyl)-pyrazolyl, 1N-(1-4C-alkyl)-triazolyl or 1N-(1-4C-alkyl)-pyrrolyl, each as defined afore and each of which is substituted by methyl or ethyl, like methyl-substituted 1N-methyl-imidazolyl (e.g. 1,4-dimethyl-imidazol-2-yl or 1,5-dimethylimidazol-2-yl), or methyl-substituted 1N-methyl-pyrazolyl (e.g. 1,3-dimethyl-pyrazol-5-yl or 1,3-dimethyl-pyrazol-4-yl).

1-4C-alkyl-substituted 1N—(H)-imidazolyl, 1-4C-alkyl-substituted 1N—(H)-pyrazolyl, 1-4C-alkyl-substituted 1N—(H)-triazolyl or 1-4C-alkyl-substituted 1N—(H)-pyrrolyl may include, for example, 1N—(H)-imidazolyl, 1N—(H)-pyrazolyl, 1N—(H)-triazolyl or 1N—(H)-pyrrolyl each as defined below and each of which is substituted on a ring carbon atom by methyl or ethyl, like methyl-substituted 1N—(H)-imidazolyl (e.g. 4-methyl-1H-imidazol-2-yl or 5-methyl-1H-imidazol-2-yl), or methyl-substituted 1N—(H)-pyrazolyl (e.g. 3-methyl-1H-pyrazol-4-yl).

1N—(H)-imidazolyl, 1N—(H)-pyrazolyl, 1N—(H)-triazolyl or 1N—(H)-pyrrolyl refers to imidazolyl, pyrazolyl, triazolyl or pyrrolyl, respectively, which is substituted by hydrogen on the nitrogen atom in position 1, such as e.g. 1H-imidazol-2-yl, 1H-imidazol-5-yl, 1H-imidazol-4-yl, 1H-pyrazol-4-yl or 1H-pyrazol-5-yl.

The term “oxo” as used herein refers to a doubly carbon-bonded oxygen atom, forming together with the carbon atom to which it is attached a carbonyl or keto group (C═O). An oxo group which is a substituent of a (hetero)aromatic ring results in a replacement of ═C(—H)— by —O(═O)— at its binding position. It will be apparent that the introduction of an oxo substituent on an (hetero)aromatic ring destroys the (hetero)aromaticity.

The term (Raa)-methyl stands for methyl which is substituted by Raa. The term 2-(Raa)-ethyl stands for ethyl which is substituted in 2-position by Raa. The term 3-(Raa)-propyl stands for propyl which is substituted in 3-position by Raa. The term 1-(Raa)-ethyl stands for ethyl which is substituted in 1-position by Raa (including (S)-1-(Raa)-ethyl and (R)-1-(Raa)-ethyl).

In general and unless otherwise mentioned, the heterocyclic radicals include all the possible isomeric forms thereof, e.g. the positional isomers thereof. Thus, for some illustrative non-restricting example, the term pyridinyl or pyridyl includes pyridin-2-yl, pyridin-3-yl and pyridin-4-yl; or the term thiophenyl or thienyl includes thiophen-2-yl and thiophen-3-yl; or the term 1N—(R14)-piperidin-2-onyl includes 1N—(R14)-piperidin-2-on-3-yl, 1N—(R14)-piperidin-2-on-4-yl, 1N—(R14)-piperidin-2-on-5-yl and 1N—(R14)-piperidin-2-on-6-yl, or the term triazol-1-yl includes [1,2,3]triazol-1-yl, [1,3,4]triazol-1-yl and [1,2,4]triazol-1-yl.

The heterocyclic groups mentioned herein refer, unless otherwise noted, to all of the possible tautomers, e.g. the keto/enol tautomers, thereof, in pure form as well as any mixtures thereof. Thus, for example, pyridine compounds which are substituted by a hydroxyl or an oxo group in the 2- or 4-position of the pyridine ring can exist in different tautomeric forms, i.e. the enol and the keto form, which are both contemplated by the present invention in pure form as well as in any mixtures thereof.

Constituents which are optionally substituted as stated herein, may be substituted, unless otherwise noted, at any possible position.

Unless otherwise noted, the carbocyclic radicals mentioned herein may be substituted by its substituents or parent molecular groups at any possible position.

The heterocyclic groups mentioned herein may be substituted by their given substituents or parent molecular groups, unless otherwise noted, at any possible position, such as e.g. at any substitutable ring carbon or ring nitrogen atom.

Unless otherwise noted, rings containing quaternizable amino- or imino-type ring nitrogen atoms (—N═) may be preferably not quaternized on these amino- or imino-type ring nitrogen atoms by the mentioned substituents or parent molecular groups.

Unless otherwise noted, any heteroatom of a heterocyclic ring with unsatisfied valences mentioned herein is assumed to have the hydrogen atom(s) to satisfy the valences.

When any variable occurs more than one time in any constituent, each definition is independent.

The person skilled in the art is aware on account of his/her expert knowledge that certain combinations of the variable characteristics mentioned in the description of this invention lead to chemically les stable compounds. This can apply, for example, to certain compounds, in which—in a manner being disadvantageous for chemical stability—two heteroatoms (S, N or O) would directly meet or would only be separated by one carbon atom. Those compounds according to this invention, in which the combination of the abovementioned variable substituents does not lead to chemically less stable compounds, are therefore preferred.

Suitable salts for compounds of formula I according to this invention—depending on substitution—are all acid addition salts or all salts with bases. Particular mention may be made of the pharmacologically tolerable inorganic and organic acids and bases customarily used in pharmacy. Those suitable are, on the one hand, water-insoluble and, particularly, water-soluble acid addition salts with acids such as, for example, hydrochloric acid (to obtain hydrochlorides), hydrobromic acid (hydrobromides), phosphoric acid (phosphates), nitric acid (nitrates), sulphuric acid (sulfates), acetic acid (acetates), citric acid (citrates), D-gluconic acid (D-gluconates), benzoic acid (benzoates), 2-(4-hydroxybenzoyl)benzoic acid [2-(4-hydroxybenzoyl)benzoates], butyric acid (butyrates), sulphosalicylic acid (sulfosalicylates), maleic acid (maleates), lauric acid (laurates), malic acid (maleates), fumaric acid (fumarates), succinic acid (succinates), oxalic acid (oxalates), tartaric acid (tartrates), embonic acid (embonates), stearic acid (stearates), toluenesulphonic acid (toluenesulfonates), methanesulphonic acid (methanesulfonates) or 3-hydroxy-2-naphthoic acid(3-hydroxy-2-naphthoates), the acids being employed in salt preparation—depending on whether a mono- or polybasic acid is concerned and depending on which salt is desired—in an equimolar quantitative ratio or one differing therefrom.

On the other hand, salts with bases are—depending on substitution—also suitable. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

Salts which are unsuitable for pharmaceutical uses but which can be employed, for example, for the isolation or purification of free compounds of formula I or their pharmaceutically acceptable salts, are also included.

Pharmacologically unacceptable salts, which can be obtained, for example, as process products during the preparation of the compounds according to this invention on an industrial scale, are converted into pharmacologically acceptable salts by processes known to the person skilled in the art.

According to expert's knowledge the compounds of formula I according to this invention as well as their salts may contain, e.g. when isolated in crystalline form, varying amounts of solvents. Included within the scope of the invention are therefore all solvates and in particular all hydrates of the compounds of formula I according to this invention as well as all solvates and in particular all hydrates of the salts of the compounds of formula I according to this invention.

In one embodiment of this invention, salts of compounds of formula I include a salt of a compound of formula I with hydrochloric acid (a hydrochloride salt).

Furthermore, the invention includes all conceivable tautomeric forms of the compounds of the present invention in pure form as well as any mixtures thereof.

In the context of this invention, hyperproliferation and analogous terms are used to describe aberrant/dysregulated cellular growth, a hallmark of diseases like cancer. This hyperproliferation might be caused by single or multiple cellular/molecular alterations in respective cells and can be, in context of a whole organism, of benign or malignant behaviour. Inhibition of cell proliferation and analogous terms is used herein to denote an ability of the compound to retard the growth of and/or kill a cell contacted with that compound as compared to cells not contacted with that compound. Most preferable this inhibition of cell proliferation is 100%, meaning that proliferation of all cells is stopped and/or cells undergo programmed cell death. In some preffered embodiments the contacted cell is a neoplastic cell. A neoplastic cell is defined as a cell with aberrant cell proliferation and/or the potential to metastasize to different tissues or organs. A benign neoplasia is described by hyperproliferation of cells, incapable of forming an aggressive, metastasizing tumor in-vivo. In contrast, a malignant neoplasia is described by cells with different cellular and biochemical abnormalities, e.g. capable of forming tumor metastasis. The acquired functional abnormalities of malignant neoplastic cells (also defined as “hallmarks of cancer”) are limitless replicative potential, self-sufficiency in growth signals, insensitivity to anti-growth signals, evasion from apoptosis, sustained angiogenesis and tissue invasion and metastasis.

Inducer of apoptosis and analogous terms are used herein to identify a compound which induces programmed cell death in cells contacted with that compound. Apoptosis is defined by complex biochemical events within the contacted cell, such as the activation of cystein specific proteinases (“caspases”) and the fragmentation of chromatin. Induction of apoptosis in cells contacted with the compound might not necessarily be coupled with inhibition of cell proliferation. Preferably, the inhibition of cell proliferation and/or induction of apoptosis is specific to cells with aberrant cell growth (hyperproliferation). Thus, compared to cells with aberrant cell growth, normal proliferating or arrested cells are less sensitive or even insensitive to the proliferation inhibiting or apoptosis inducing activity of the compound. Finally, cytotoxic is used in a more general sense to identify compounds which kill cells by various mechanisms, including the induction of apoptosis/programmed cell death in a cell cycle dependent or cell-cycle independent manner.

Cell cycle specific and analogous terms are used herein to identify a compound as inducing apoptosis/killing only in proliferating cells actively passing a specific phase of the cell cycle, but not in resting, non-dividing cells. Continuously proliferating cells are typical for diseases like cancer and characterized by cells passing all phases of the cell division cycle, namely in the G (“gap”) 1, S (“DNA synthesis”), G2 and M (“mitosis”) phase.

Compounds according to the present invention more worthy to be mentioned include those compounds of formula I

wherein
Ra is —C(O)—N(R11)-R1, in which
R1 is 1-4C-alkyl, 3-6C-cycloalkyl, HetA, phenyl, HarA, 1-3C-alkyl, such as e.g. methyl, ethyl or propyl, which is substituted by Raa, or 3-4C-alkyl, such as e.g. propyl or butyl, which is substituted by Rab and Rac on different carbon atoms,
   wherein said 3-6C-cycloalkyl may be optionally substituted by one or two substituents independently selected from R12, and
   wherein each of said phenyl and HarA may be optionally substituted by one, two or three substituents independently selected from R13,
R11 is hydrogen or methyl,
or R1 and R11 together and with inclusion of the nitrogen atom, to which they are attached, form a heterocyclic radical HET, in which
   either
HET is piperidin-1-yl, pyrrolidin-1-yl, morpholin-4-yl, thiomorpholin-4-yl or 4N-(1-2C-alkylcarbonyl)-piperazin-1-yl,
   or
HET is pyrrol-1-yl, imidazol-1-yl, pyrazol-1-yl or triazol-1-yl,
Rb is -T-Q, in which
T is a ethane-1,2-diyl, trans-cyclopropane-1,2-diyl, or propane-1,2-diyl bridge, and
   either
Q is optionally substituted by Rba and/or Rbb, and is phenyl,
   or
Q is optionally substituted by Rca and/or Rcb, and is pyridyl,
   or
Q is optionally substituted by Rda and/or Rdb, and is furyl or thienyl,
   or
Q is optionally substituted by Rea and/or Reb, and is cyclohexyl or cyclopentyl,
   wherein
Raa is selected from the group consisting of:
   3-6C-cycloalkyl, phenyl,
   hydroxyl,
   HarB, HetB, HetC, morpholino,
   —C(O)OR3, —N(R4)R5,
   —OC(O)R8, and
   —OR9,
   wherein said 3-6C-cycloalkyl may be optionally substituted by one or two substituents independently selected from R12, and
   wherein each of said phenyl and HarB may be optionally substituted by one, two or three substituents independently selected from R13,
   in which
R3, R4 and R5 may be the same or different and are independently selected from the group consisting of: hydrogen, and 1-4C-alkyl such as e.g. methyl or ethyl,
R8 is 1-4C-alkyl such as e.g. methyl,
R9 is selected from the group consisting of:
   1-4C-alkyl such as e.g. methyl, ethyl, propyl or isopropyl,
   phenyl-1-2C-alkyl such as e.g. benzyl,
   1-2C-alkoxy-2-3C-alkyl such as e.g. 2-methoxyethyl, and
   (1-4C-alkoxy-2-4C-alkoxy)-2-4C-alkyl such as e.g. 2-(2-methoxyethoxy)-ethyl,
      either
HarA is bonded to the parent molecular group via a ring carbon atom, and is a 5-membered monocyclic partially unsaturated or aromatic heterocyclic ring comprising one to four heteroatoms independently selected from nitrogen, oxygen and sulphur,
   or
HarA is bonded to the parent molecular group via a ring carbon atom, and is a 6-membered monocyclic partially unsaturated or aromatic heterocyclic ring comprising one or two nitrogen atoms,
   or
HarA is bonded to the parent molecular group via a ring carbon atom, and is a 5-membered monocyclic partially unsaturated or aromatic heterocyclic ring comprising one to three heteroatoms independently selected from nitrogen, oxygen and sulphur, which heterocyclic ring is substituted by one oxo group,
   or
HarA is bonded to the parent molecular group via a ring carbon atom, and is a 6-membered monocyclic partially unsaturated or aromatic heterocyclic ring comprising one or two nitrogen atoms, which heterocyclic ring is substituted by one oxo group,
   either
HarB is bonded to the parent molecular group via a ring carbon or a ring nitrogen atom, and is a 5-membered monocyclic partially unsaturated or aromatic heterocyclic ring comprising one to four heteroatoms independently selected from nitrogen, oxygen and sulphur,
   or
HarB is bonded to the parent molecular group via a ring carbon atom, and is a 6-membered monocyclic partially unsaturated or aromatic heterocyclic ring comprising one or two nitrogen atoms,
   or
HarB is bonded to the parent molecular group via a ring carbon or a ring nitrogen atom, and is a 5-membered monocyclic partially unsaturated or aromatic heterocyclic ring comprising one to three heteroatoms independently selected from nitrogen, oxygen and sulphur, which heterocyclic ring is substituted by one oxo group,
   or
HarB is bonded to the parent molecular group via a ring carbon or a ring nitrogen atom, and is a 6-membered monocyclic partially unsaturated or aromatic heterocyclic ring comprising one or two nitrogen atoms, which heterocyclic ring is substituted by one oxo group,
each R12 may be the same or different and is independently selected from the group consisting of:
   methyl, ethyl, halogen, hydroxyl, methoxy, and ethoxy,
each R13 may be the same or different and is independently selected from the group consisting of:
   methyl, ethyl, halogen, hydroxyl, methoxy, ethoxy, amino, aminomethyl, mono- or di-1-2C-alkylamino, hydroxy-2-3C-alkoxy, 1-3C-alkoxy-2-3C-alkoxy, hydroxy-1-2C-alkyl, and 1-3C-alkoxy-1-2C-alkyl,
HetA is bonded to the parent molecular group via a ring carbon atom, and is tetrahydropyranyl, tetrahydrofuryl, 1N-(1-2C-alkylcarbonyl)-piperidinyl, 1N-(1-2C-alkylcarbonyl)-pyrrolidinyl, 1N-(formyl)-piperidinyl, 1N-(formyl)-pyrrolidinyl, tetrahydrothiapyranyl, tetrahydrothienyl, 1N—(R14)-piperidin-2-onyl, 1N—(R14)-pyrrolidin-2-onyl, tetrahydropyran-2-onyl, tetrahydrofuran-2-onyl, 3N—(R14)-oxazolidin-2-onyl, or 1N—(R14)-3N—(R15)-imidazolidin-2-onyl, wherein each of said HetA may be optionally substituted by one or two substituents independently selected from R16,
HetB is bonded to the parent molecular group via a ring nitrogen atom, and is piperidin-2-on-1-yl, pyrrolidin-2-on-1-yl, oxazolidin-2-on-1-yl, or 3N—(R15)-imidazolidin-2-on-1-yl, wherein each of said HetB may be optionally substituted by one or two substituents independently selected from R16,
HetC is bonded to the parent molecular group via a ring carbon atom, and is tetrahydropyranyl, tetrahydrofuryl, 1N-(1-2C-alkylcarbonyl)-piperidinyl, 1N-(1-2C-alkylcarbonyl)-pyrrolidinyl, 1N-(formyl)-piperidinyl, 1N-(formyl)-pyrrolidinyl, tetrahydrothiapyranyl, tetrahydrothienyl, 1N—(R14)-piperidin-2-onyl, 1N—(R14)-pyrrolidin-2-onyl, tetrahydropyran-2-onyl, tetrahydrofuran-2-onyl, 3N—(R14)-oxazolidin-2-onyl, or 1N—(R14)-3N—(R15)-imidazolidin-2-onyl, wherein each of said HetC may be optionally substituted by one or two substituents independently selected from R16, in which
R14 is hydrogen, methyl, ethyl, propyl or isopropyl,
R15 is hydrogen, methyl, ethyl, propyl or isopropyl,
each R16 may be the same or different and is independently selected from the group consisting of:
   methyl, ethyl, halogen, hydroxyl, methoxy, and ethoxy,
Rab is hydroxyl,
Rac is hydroxyl,
or Rab and Rac bonded to adjacent carbon atoms form together a dimethylmethylenedioxy bridge,
Rba is methyl, ethyl, methoxy, ethoxy or halogen,
Rbb is methyl, ethyl, methoxy, ethoxy or halogen,
Rca is methyl, ethyl, methoxy, ethoxy or halogen,
Rcb is methyl, ethyl, methoxy, ethoxy or halogen,
Rda is methyl, ethyl or halogen,
Rdb is methyl, ethyl or halogen,
Rea is methyl, ethyl, methoxy, ethoxy, halogen or hydroxyl,
Reb is methyl, ethyl, methoxy, ethoxy, halogen or hydroxyl;
   in particular
   either
Q is phenyl,
   or
Q is 2-methoxyphenyl, 2-ethoxyphenyl, 3-methoxyphenyl, 2-methoxy-5-methyl-phenyl or 2-ethoxy-5-methyl-phenyl,
   or
Q is pyridin-2-yl or pyridin-3-yl,
   or
Q is furan-2-yl, furan-3-yl, thiophen-2-yl or thiophen-3-yl,
   or
Q is cyclohexyl or cyclopentyl;
   in more particular
   either
Q is phenyl,
   or
Q is 2-methoxyphenyl, 2-ethoxyphenyl or 3-methoxyphenyl,
   or
Q is pyridin-2-yl or pyridin-3-yl,
   or
Q is furan-2-yl,
   or
Q is cyclohexyl or cyclopentyl; and the salts, as well as the stereoisomers and salts of the stereoisomers thereof.

Compounds according to the present invention in particular worthy to be mentioned include those compounds of formula I

wherein
Ra is —C(O)—N(R11)-R1, in which
   either
R1 is methyl, ethyl, propyl, isopropyl or isobutyl,
   or
R1 is cyclopropyl, cyclobutyl, cyclopentyl or cyclohexyl,
wherein each of said cyclopropyl, cyclobutyl, cyclopentyl and cyclohexyl may be optionally substituted by one or two substituents independently selected from R12,
   or
R1 is phenyl,
wherein said phenyl may be optionally substituted by one or two substituents independently selected from R13,
   or
R1 is HarA, in which
   either
HarA is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
   or
HarA is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
   or
HarA is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
   or
HarA is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
   or
HarA is pyridyl or pyrimidinyl,
wherein each of said HarA may be optionally substituted by one or two substituents independently selected from R13,
   or
R1 is HetA, in which
HetA is tetrahydropyranyl, tetrahydrofuryl, tetrahydropyran-2-onyl, tetrahydrofuran-2-onyl, 1N-(acetyl)-piperidinyl, 1N-(acetyl)-pyrrolidinyl, 1N-(formyl)-piperidinyl, 1N-(formyl)-pyrrolidinyl, 1N-(methyl)-piperidin-2-onyl, 1N-(methyl)-pyrrolidin-2-onyl, 1N—(H)-piperidin-2-onyl or 1N—(H)-pyrrolidin-2-onyl, wherein each of said tetrahydropyranyl and tetrahydrofuranyl may be optionally substituted by one or two substituents independently selected from R16,
   or
R1 is (Raa)-methyl, 2-(Raa)-ethyl or 1-(Raa)-ethyl, in which
Raa is cyclopropyl, cyclobutyl, cyclopentyl or cyclohexyl,
wherein each of said cyclopropyl, cyclobutyl, cyclopentyl and cyclohexyl may be optionally substituted by one or two substituents independently selected from R12,
   or
R1 is (Raa)-methyl, 2-(Raa)-ethyl or 1-(Raa)-ethyl, in which
Raa is phenyl,
wherein said phenyl may be optionally substituted by one or two substituents independently selected from R13,
   or
R1 is (Raa)-methyl, 2-(Raa)-ethyl or 1-(Raa)-ethyl, in which
Raa is HarB, in which
   either
HarB is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
   or
HarB is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
   or
HarB is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
   or
HarB is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
   or
HarB is pyridyl or pyrimidinyl,
wherein each of said HarB may be optionally substituted by one or two substituents independently selected from R13,
   or
R1 is (Raa)-methyl, 2-(Raa)-ethyl or 1-(Raa)-ethyl, in which
Raa is HetC, in which
HetC is tetrahydropyranyl, tetrahydrofuryl, tetrahydropyran-2-onyl, tetrahydrofuran-2-onyl, 1N-(acetyl)-piperidinyl, 1N-(acetyl)-pyrrolidinyl, 1N-(formyl)-piperidinyl, 1N-(formyl)-pyrrolidinyl, 1N-(methyl)-piperidin-2-onyl, 1N-(methyl)-pyrrolidin-2-onyl, 1N—(H)-piperidin-2-onyl, 1N—(H)-pyrrolidin-2-onyl, 3N-(methyl)-oxazolidin-2-onyl, 3N—(H)-oxazolidin-2-onyl, 1N-(methyl)-3N—(H)-imidazolidin-2-onyl, 1N-(methyl)-3N-(methyl)-imidazolidin-2-onyl, or 1N—(H)-3N—(H)-imidazolidin-2-onyl,
wherein each of said tetrahydropyranyl and tetrahydrofuranyl may be optionally substituted by one or two substituents independently selected from R16,
   or
R1 is 2-(Raa)-ethyl, in which
Raa is hydroxyl or —OR9, in which
R9 is methyl, ethyl, 2-methoxyethyl or 2-(2-methoxyethoxy)-ethyl,
   or
R1 is 2-(Raa)-ethyl, in which
Raa is HarB, in which
HarB is imidazol-1-yl, pyrazol-1-yl, triazol-1-yl, mono- or di-(1-2C-alkyl)-substituted imidazol-1-yl, mono- or di-(1-2C-alkyl)-substituted pyrazol-1-yl, or mono- or di-(1-2C-alkyl)-substituted triazol-1-yl, wherein each of said HarB may be optionally substituted by one or two substituents independently selected from R13,
   or
R1 is 2,3-dihydroxy-propyl,
R11 is hydrogen,
Rb is -T-Q, in which
T is a ethane-1,2-diyl, trans-cyclopropane-1,2-diyl, or propane-1,2-diyl bridge, and
   either
Q is optionally substituted by Rba and/or Rbb, and is phenyl,
   or
Q is optionally substituted by Rca and/or Rcb, and is pyridyl,
   or
Q is optionally substituted by Rda and/or Rdb, and is furyl or thienyl,
   or
Q is optionally substituted by Rea and/or Reb, and is cyclohexyl or cyclopentyl,
   wherein
each R12 may be the same or different and is independently selected from the group consisting of:
   methyl, ethyl, fluorine, chlorine, hydroxyl, and methoxy,
each R13 may be the same or different and is independently selected from the group consisting of:
   methyl, ethyl, fluorine, chlorine, hydroxyl, methoxy, amino, aminomethyl, mono- or dimethylamino, 2-hydroxy-ethoxy, 2-(1-2C-alkoxy)-ethoxy, hydroxy-1-2C-alkyl, and (1-2C-alkoxy)-1-2C-alkyl,
each R16 may be the same or different and is independently selected from the group consisting of:
   methyl, ethyl, fluorine, chlorine, hydroxyl, and methoxy,
Rba is methyl, methoxy, ethoxy, fluorine, chlorine or bromine,
Rbb is methyl, methoxy, ethoxy, fluorine, chlorine or bromine,
Rca is methyl, methoxy, ethoxy, fluorine, chlorine or bromine,
Rcb is methyl, methoxy, ethoxy, fluorine, chlorine or bromine,
Rda is methyl, fluorine, chlorine or bromine,
Rdb is methyl, fluorine, chlorine or bromine,
Rea is methyl, methoxy, ethoxy, fluorine, chlorine, or hydroxyl,
Reb is methyl, methoxy, ethoxy, fluorine, chlorine or hydroxyl;
   in particular
   either
Q is phenyl,
   or
Q is 2-methoxyphenyl, 2-ethoxyphenyl, 3-methoxyphenyl, 2-methoxy-5-methyl-phenyl or 2-ethoxy-5-methyl-phenyl,
   or
Q is pyridin-2-yl or pyridin-3-yl,
   or
Q is furan-2-yl, furan-3-yl, thiophen-2-yl or thiophen-3-yl,
   or
Q is cyclohexyl or cyclopentyl;
   in more particular
   either
Q is phenyl,
   or
Q is 2-methoxyphenyl, 2-ethoxyphenyl or 3-methoxyphenyl,
   or
Q is pyridin-2-yl or pyridin-3-yl,
   or
Q is furan-2-yl,
   or
Q is cyclohexyl or cyclopentyl; and the salts, as well as the stereoisomers and salts of the stereoisomers thereof.

Compounds according to the present invention in more particular worthy to be mentioned include those compounds of formula I

wherein
Ra is —C(O)—N(R11)-R1, in which
   either
R1 is methyl, ethyl, propyl, isopropyl or isobutyl,
   or
R1 is cyclopropyl, cyclobutyl, cyclopentyl or cyclohexyl,
wherein each of said cyclopropyl, cyclobutyl, cyclopentyl and cyclohexyl may be optionally substituted by one or two substituents independently selected from R12,
   or
R1 is HarA, in which
   either
HarA is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
   or
HarA is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
   or
HarA is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
   or
HarA is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
   or
HarA is pyridyl,
wherein said pyridyl may be optionally substituted by one or two substituents independently selected from R13,
   or
R1 is HetA, in which
HetA is tetrahydropyranyl, tetrahydrofuryl, tetrahydropyran-2-onyl, tetrahydrofuran-2-onyl, 1N-(acetyl)-piperidinyl, 1N-(acetyl)-pyrrolidinyl, 1N-(formyl)-piperidinyl, 1N-(formyl)-pyrrolidinyl, 1N-(methyl)-piperidin-2-onyl, 1N-(methyl)-pyrrolidin-2-onyl, 1N—(H)-piperidin-2-onyl or 1N—(H)-pyrrolidin-2-onyl,
   or
R1 is (Raa)-methyl, 2-(Raa)-ethyl or 1-(Raa)-ethyl, in which
Raa is cyclopropyl, cyclobutyl, cyclopentyl or cyclohexyl,
wherein each of said cyclopropyl, cyclobutyl, cyclopentyl and cyclohexyl may be optionally substituted by one or two substituents independently selected from R12,
   or
R1 is (Raa)-methyl, 2-(Raa)-ethyl or 1-(Raa)-ethyl, in which
Raa is phenyl,
wherein said phenyl may be optionally substituted by one or two substituents independently selected from R13,
   or
R1 is (Raa)-methyl, 2-(Raa)-ethyl or 1-(Raa)-ethyl, in which
Raa is HarB, in which
   either
HarB is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
   or
HarB is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
   or
HarB is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
   or
HarB is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
   or
HarB is pyridyl,
   or
R1 is (Raa)-methyl, 2-(Raa)-ethyl or 1-(Raa)-ethyl, in which
Raa is HetC, in which
HetC is tetrahydropyranyl, tetrahydrofuryl, tetrahydropyran-2-onyl, tetrahydrofuran-2-onyl, 1N-(acetyl)-piperidinyl, 1N-(acetyl)-pyrrolidinyl, 1N-(formyl)-piperidinyl, 1N-(formyl)-pyrrolidinyl, 1N-(methyl)-piperidin-2-onyl, 1N-(methyl)-pyrrolidin-2-onyl, 1N—(H)-piperidin-2-onyl, 1N—(H)-pyrrolidin-2-onyl, 3N-(methyl)-oxazolidin-2-onyl or 3N—(H)-oxazolidin-2-onyl,
   or
R1 is 2-(Raa)-ethyl, in which
Raa is hydroxyl or —OR9, in which
R9 is methyl, ethyl or 2-methoxyethyl,
   or
R1 is 2-(Raa)-ethyl, in which
Raa is HarB, in which
HarB is imidazol-1-yl, pyrazol-1-yl, triazol-1-yl, mono- or di-(1-2C-alkyl)-substituted imidazol-1-yl, mono- or di-(1-2C-alkyl)-substituted pyrazol-1-yl, or mono- or di-(1-2C-alkyl)-substituted triazol-1-yl,
   or
R1 is 2,3-dihydroxy-propyl,
R11 is hydrogen,
Rb is -T-Q, in which
T is a ethane-1,2-diyl, trans-cyclopropane-1,2-diyl, or propane-1,2-diyl bridge, and
   either
Q is optionally substituted by Rba and/or Rbb, and is phenyl,
   or
Q is optionally substituted by Rca and/or Rcb, and is pyridyl,
   or
Q is optionally substituted by Rda and/or Rdb, and is furyl or thienyl,
   or
Q is optionally substituted by Rea and/or Reb, and is cyclohexyl or cyclopentyl,
   wherein
each R12 may be the same or different and is independently selected from the group consisting of:
   methyl, fluorine, hydroxyl, and methoxy,
each R13 may be the same or different and is independently selected from the group consisting of:
   methyl, fluorine, hydroxyl, and methoxy,
Rba is methyl, methoxy, ethoxy, fluorine, chlorine or bromine,
Rbb is methyl, methoxy, ethoxy, fluorine, chlorine or bromine,
Rca is methyl, methoxy, ethoxy, fluorine, chlorine or bromine,
Rcb is methyl, methoxy, ethoxy, fluorine, chlorine or bromine,
Rda is methyl, fluorine, chlorine or bromine,
Rdb is methyl, fluorine, chlorine or bromine,
Rea is methyl, methoxy, ethoxy, fluorine, chlorine or hydroxyl,
Reb is methyl, methoxy, ethoxy, fluorine, chlorine or hydroxyl;
   in particular
   either
Q is phenyl,
   or
Q is 2-methoxyphenyl, 2-ethoxyphenyl, 3-methoxyphenyl, 2-methoxy-5-methyl-phenyl or 2-ethoxy-5-methyl-phenyl,
   or
Q is pyridin-2-yl or pyridin-3-yl,
   or
Q is furan-2-yl, furan-3-yl, thiophen-2-yl or thiophen-3-yl,
   or
Q is cyclohexyl or cyclopentyl;
   in more particular
   either
Q is phenyl,
   or
Q is 2-methoxyphenyl, 2-ethoxyphenyl or 3-methoxyphenyl,
   or
Q is pyridin-2-yl or pyridin-3-yl,
   or
Q is furan-2-yl,
   or
Q is cyclohexyl or cyclopentyl;
and the salts, as well as the stereoisomers and salts of the stereoisomers thereof.
   Compounds according to the present invention to be emphasized include those compounds of formula I
   wherein
Ra is —C(O)—N(R11)-R1, in which
   either
R1 is methyl or ethyl,
   or
R1 is cyclopropyl, cyclobutyl, cyclopentyl or cyclohexyl,
   or
R1 is HetA, in which
HetA is tetrahydropyranyl or tetrahydrofuryl,
   or
R1 is (Raa)-methyl, 2-(Raa)-ethyl or 1-(Raa)-ethyl, in which
Raa is cyclopropyl, cyclobutyl, cyclopentyl or cyclohexyl,
   or
R1 is (Raa)-methyl, 2-(Raa)-ethyl or 1-(Raa)-ethyl, in which
Raa is phenyl,
   or
R1 is (Raa)-methyl, 2-(Raa)-ethyl or 1-(Raa)-ethyl, in which
Raa is HarB, in which
   either
HarB is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
   or
HarB is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
   or
HarB is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
   or
HarB is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
   or
HarB is pyridyl,
   or
R1 is (Raa)-methyl, 2-(Raa)-ethyl or 1-(Raa)-ethyl, in which
Raa is HetC, in which
HetC is tetrahydropyranyl, tetrahydrofuryl, tetrahydropyran-2-onyl, tetrahydrofuran-2-onyl, 1N-(acetyl)-piperidinyl, 1N-(acetyl)-pyrrolidinyl, 1N-(formyl)-piperidinyl, 1N-(formyl)-pyrrolidinyl, 1N-(methyl)-piperidin-2-onyl, 1N-(methyl)-pyrrolidin-2-onyl, 1N—(H)-piperidin-2-onyl, 1N—(H)-pyrrolidin-2-onyl, 3N-(methyl)-oxazolidin-2-onyl or 3N—(H)-oxazolidin-2-onyl,
   or
R1 is 2-(Raa)-ethyl, in which
Raa is hydroxyl or —OR9, in which
R9 is methyl, ethyl or 2-methoxyethyl,
   or
R1 is 2-(Raa)-ethyl, in which
Raa is HarB, in which
HarB is imidazol-1-yl, pyrazol-1-yl, triazol-1-yl, mono- or di-(1-2C-alkyl)-substituted imidazol-1-yl, mono- or di-(1-2C-alkyl)-substituted pyrazol-1-yl, or mono- or di-(1-2C-alkyl)-substituted triazol-1-yl,
   or
R1 is 2,3-dihydroxy-propyl,
R11 is hydrogen,
Rb is -T-Q, in which
T is a ethane-1,2-diyl, trans-cyclopropane-1,2-diyl, or propane-1,2-diyl bridge, and
   either
Q is phenyl,
   or
Q is 2-methoxyphenyl, 2-ethoxyphenyl, 3-methoxyphenyl, 2-methoxy-5-methyl-phenyl or 2-ethoxy-5-methyl-phenyl,
   or
Q is pyridin-2-yl or pyridin-3-yl,
   or
Q is furan-2-yl, furan-3-yl, thiophen-2-yl or thiophen-3-yl,
   or
Q is cyclohexyl or cyclopentyl;
   in particular
   either
Q is phenyl,
   or
Q is 2-methoxyphenyl, 2-ethoxyphenyl or 3-methoxyphenyl,
   or
Q is pyridin-2-yl or pyridin-3-yl,
   or
Q is furan-2-yl,
   or
Q is cyclohexyl;
   and the salts, as well as the stereoisomers and salts of the stereoisomers thereof.

In the compounds of formula I according to the present invention (as well as in the salts, stereoisomers and salts of the stereoisomers thereof), the significances of the following special embodiments are of concern individually or in any possible single or multiple combination thereof:

A special embodiment of the compounds of formula I according to this invention refers to those compounds of formula I, in which Ra is —C(O)—N(H)—R1.

Another special embodiment of the compounds of formula I according to this invention refers to those compounds of formula I, in which Ra is —C(O)—N(H)—CH₃.

Another special embodiment of the compounds of formula I according to this invention refers to those compounds of formula I, in which Ra is —C(O)—N(H)—CH₂CH₃.

Another special embodiment of the compounds of formula I according to this invention refers to those compounds of formula I, in which Ra is —C(O)—N(H)-cyclopropyl.

Another special embodiment of the compounds of formula I according to this invention refers to those compounds of formula I, in which Ra is —C(O)—N(H)—CH₂-cyclopropyl.

Another special embodiment of the compounds of formula I according to this invention refers to those compounds of formula I, in which Ra is —C(O)—N(H)—R1, in which
R1 is (Raa)-methyl, 2-(Raa)-ethyl or 1-(Raa)-ethyl, in which
Raa is HarB, in which
   either
HarB is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
   or
HarB is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
   or
HarB is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
   or
HarB is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
   or
HarB is pyridyl.

Another special embodiment of the compounds of formula I according to this invention refers to those compounds of formula I, in which Ra is any one of the meanings indicated in Table 1 given below.

Another special embodiment of the compounds of formula I according to this invention refers to those compounds of formula Ia, in which Q is 2-ethoxy-phenyl.

Another special embodiment of the compounds of formula I according to this invention refers to those compounds of formula Ia, in which Q is 2-methoxy-phenyl.

Another special embodiment of the compounds of formula I according to this invention refers to those compounds of formula Ia, in which Q is 3-methoxy-phenyl.

Another special embodiment of the compounds of formula I according to this invention refers to those compounds of formula Ia, in which Q is 2-methoxy-5-methyl-phenyl.

Another special embodiment of the compounds of formula I according to this invention refers to those compounds of formula Ia, in which Q is pyridin-3-yl.

Another special embodiment of the compounds of formula I according to this invention refers to those compounds of formula Ia, in which Q is pyridin-2-yl.

Another special embodiment of the compounds of formula I according to this invention refers to those compounds of formula Ia, in which Q is furan-2-yl.

Another special embodiment of the compounds of formula I according to this invention refers to those compounds of formula Ia, in which Q is phenyl.

Another special embodiment of the compounds of formula I according to this invention refers to those compounds of formula Ia, in which Q is cyclohexyl.

Another special embodiment of the compounds of formula I according to this invention refers to those compounds of formula Ib, in which Q is 2-ethoxy-phenyl.

Another special embodiment of the compounds of formula I according to this invention refers to those compounds of formula Ib, in which Q is 2-methoxy-phenyl.

Another special embodiment of the compounds of formula I according to this invention refers to those compounds of formula Ib, in which Q is 3-methoxy-phenyl.

Another special embodiment of the compounds of formula I according to this invention refers to those compounds of formula Ib, in which Q is pyridin-3-yl.

Another special embodiment of the compounds of formula I according to this invention refers to those compounds of formula Ib, in which Q is pyridin-2-yl.

Another special embodiment of the compounds of formula I according to this invention refers to those compounds of formula Ib, in which Q is furan-2-yl.

Another special embodiment of the compounds of formula I according to this invention refers to those compounds of formula Ib, in which Q is phenyl.

Another special embodiment of the compounds of formula I according to this invention refers to those compounds of formula Ic, in which Q is 2-ethoxy-phenyl.

Another special embodiment of the compounds of formula I according to this invention refers to those compounds of formula Ic, in which Q is 2-methoxy-phenyl.

Another special embodiment of the compounds of formula I according to this invention refers to those compounds of formula Ic, in which Q is 3-methoxy-phenyl.

Another special embodiment of the compounds of formula I according to this invention refers to those compounds of formula Ic, in which Q is pyridin-3-yl.

Another special embodiment of the compounds of formula I according to this invention refers to those compounds of formula Ic, in which Q is pyridin-2-yl

Another special embodiment of the compounds of formula I according to this invention refers to those compounds of formula Ic, in which Q is furan-2-yl.

Another special embodiment of the compounds of formula I according to this invention refers to those compounds of formula Ic, in which Q is phenyl.

Another special embodiment of the compounds of formula I according to this invention refers to those compounds of formula Ic, in which Q is cyclohexyl.

It is to be understood that the present invention includes any or all possible combinations and subsets of the special embodiments defined hereinabove.
Numbering:

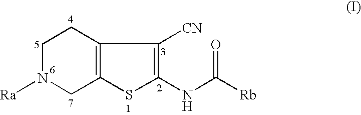

An embodimental variant (variant a1) of the compounds of formula I according to this invention includes those compounds of formula I, which are from formula Ia, and the salts, stereoisomers and salts of the stereoisomers thereof:

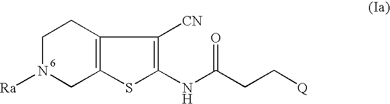

A further embodimental variant (variant b1) of the compounds of formula I according to this invention includes those compounds of formula I, which are from formula Ib, and the salts, stereoisomers and salts of the stereoisomers thereof:

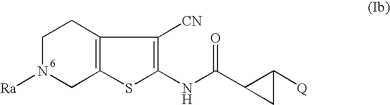

In the context of variant b1, one subvariant of variant b1 includes compounds of formula Ib, in which the radicals —N(H)—C(O)— and Q are located at the opposite side of the plane defined by the cyclopropane ring (trans configuration). A more precise subvariant of variant b1 includes compounds of formula Ib*, another more precise subvariant of variant b includes compounds of formula Ib**, as well as the salts thereof:

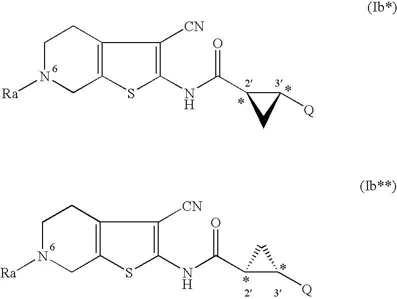

If, for example, in compounds of formula Ib* Q is optionally substituted phenyl, pyridyl, furyl or thienyl as defined above, then the configuration—according the rules of Cahn, Ingold and Prelog—is R in the position 2′ and R in the position 3′ as indicated in formula Ib* above.

If, for example, in compounds of formula Ib** Q is optionally substituted phenyl, pyridyl, furyl or thienyl as defined above, then the configuration—according the rules of Cahn, Ingold and Prelog—is S in the position 2′ and S in the position 3′ as indicated in formula Ib** above.

A yet further embodimental variant (variant c1) of the compounds of formula I according to this invention includes those compounds of formula I, which are from formula Ic, and the salts, stereoisomers and salts of the stereosimers thereof:

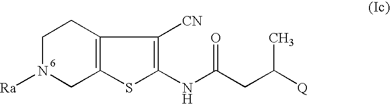

In the context of variant c1, one subvariant of variant c1 includes compounds of formula Ic*, another subvariant of variant c includes compounds of formula Ic**, as well as the salts thereof:

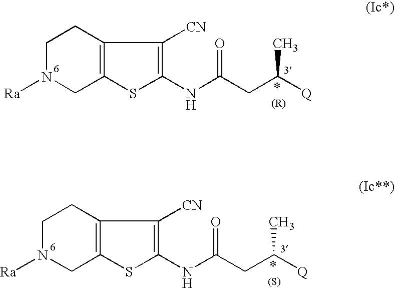

If, for example, in compounds of formula Ic* Q has one of the meanings given above, then the configuration—according the rules of Cahn, Ingold and Prelog—is R in the position 3′ as indicated in formula Ic* above.

If, for example, in compounds of formula Ic** Q has one of the meanings given above, then the configuration—according the rules of Cahn, Ingold and Prelog—is S in the position 3′ as indicated in formula Ic** above.

When the compounds of formula I are chiral compounds (e.g. by having one or more chiral centers), the invention includes all conceivable stereoisomers of the compounds of this invention, like e.g. diastereomers and enantiomers, in substantially pure form as well as in any mixing ratio, including the racemates, as well as the salts thereof.

Thus, substantially pure stereoisomers of the compounds according to this invention, particularly substantially pure stereoisomers of the following examples, are all part of the present invention and may be obtained according to procedures customary to the skilled person, e.g. by separation of corresponding mixtures, by using stereochemically pure starting materials and/or by stereoselective synthesis.

Accordingly, the stereoisomers of formula Ic* and of formula Ic** and the salts thereof are part of the invention. Likewise, the stereoisomers of formula Ib* and of formula Ib** and the salts thereof are part of the invention.

In general, enantiomerically pure compounds of this invention may be prepared according to art-known processes, such as e.g. via asymmetric syntheses, for example by preparation and separation of appropriate diastereoisomeric compounds/intermediates, which can be separated by known methods (e.g. by chromatographic separation or (fractional) crystallization from a suitable solvent), or by using chiral synthons or chiral reagents; by chromatographic separation of the corresponding racemic compounds on chiral separating columns; by means of diastereomeric salt formation of the racemic compounds with optically active acids (such as e.g. those mentioned below) or bases, subsequent resolution of the salts and release of the desired compound from the salt; by derivatization of the racemic compounds with chiral auxiliary reagents, subsequent diastereomer separation and removal of the chiral auxiliary group; by resolution via diastereomeric inclusion compounds (e.g. complexes or clathrates); by kinetic resolution of a racemate (e.g. by enzymatic resolution); by enantioselective (preferential) crystallization (or crystallization by entrainment) from a conglomerate of enantiomorphous crystals under suitable conditions; or by (fractional) crystallization from a suitable solvent in the presence of a chiral auxiliary.

Thus, e.g. one possible alternative for enantiomer separation may be carried out at the stage of the compounds of formula I or of the starting compounds having a protonatable group. Hereby, separation of the enantiomers may be carried out, for example, by means of salt formation of the racemic compounds with optically active acids, especially carboxylic acids, subsequent resolution of the salts and release of the desired compound from the salt. Examples of optically active acids which may be mentioned in this connection, without being restricted thereto, are the enantiomeric forms of mandelic acid, tartaric acid, O,O′-dibenzoyltartaric acid, camphoric acid, quinic acid, glutamic acid, pyroglutamic acid, malic acid, camphorsulfonic acid, 3-bromocamphorsulfonic acid, α-methoxyphenylacetic acid, α-methoxy-α-trifluoromethylphenylacetic acid or 2-phenylpropionic acid or the like.

Another possible alternative for enantiomer separation may be carried out by chromatographic separation of a racemic mixture of compounds of formula I or of starting compounds thereof on a chiral separating column using the appropriate separation conditions.

As illustrative compounds according to this invention the following compounds of formula Ia, in which Q is 2-ethoxy-phenyl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ia, in which Q is 3-methoxy-phenyl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ia, in which Q is pyridin-3-yl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ia, in which Q is phenyl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ia, in which Q is 2-methoxy-phenyl, and the salts as well as the stereoisomers and salts of the stereo-isomers thereof, may be mentioned by means of the substituent meanings 1) to 72) for Ra indicated in Table 1 given below.

As other illustrative compounds according to this invention the following compounds of formula Ia, in which Q is cyclohexyl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ia, in which Q is furan-2-yl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ia, in which Q is pyridin-2-yl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ia, in which Q is 2-methoxy-5-methyl-phenyl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ib, in which Q is 2-methoxy-phenyl, and the salts as well as the stereoisomers and salts of the stereo-isomers thereof, may be mentioned by means of the substituent meanings 1) to 72) for Ra indicated in Table 1 given below.

As other illustrative compounds according to this invention the following compounds of formula Ib, in which Q is 2-ethoxy-phenyl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ib, in which Q is 3-methoxy-phenyl, and the salts as well as the stereoisomers and salts of the stereo-isomers thereof, may be mentioned by means of the substituent meanings 1) to 72) for Ra indicated in Table 1 given below.

As other illustrative compounds according to this invention the following compounds of formula Ib, in which Q is phenyl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ib, in which Q is pyridin-2-yl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ib, in which Q is pyridin-3-yl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ib, in which Q is furan-2-yl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ic, in which Q is phenyl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ic, in which Q is 3-methoxy-phenyl, and the salts as well as the stereoisomers and salts of the stereo-isomers thereof, may be mentioned by means of the substituent meanings 1) to 72) for Ra indicated in Table 1 given below.

As other illustrative compounds according to this invention the following compounds of formula Ic, in which Q is pyridin-2-yl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ic, in which Q is pyridin-3-yl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ic, in which Q is 2-methoxy-phenyl, and the salts as well as the stereoisomers and salts of the stereo-isomers thereof, may be mentioned by means of the substituent meanings 1) to 72) for Ra indicated in Table 1 given below.

As other illustrative compounds according to this invention the following compounds of formula Ic, in which Q is 2-ethoxy-phenyl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ic, in which Q is furan-2-yl, and the salts as well as the stereoisomers and salts of the stereoisomers thereof, may be mentioned by means of the substituent meanings 1) to 72) for Ra indicated in Table 1 given below.

As other illustrative compounds according to this invention the following compounds of formula Ic, in which Q is cyclohexyl, and the salts as well as the stereoisomers and salts of the stereoisomers thereof, may be mentioned by means of the substituent meanings 1) to 72) for Ra indicated in Table 1 given below.

TABLE 1

| | Ra |
|---|---|
| 1) | 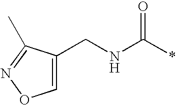 |
| 2) | 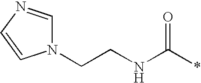 |
| 3) | 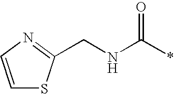 |
| 4) | 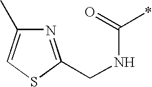 |
| 5) | 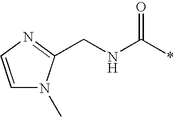 |
| 6) | 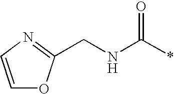 |
| 7) | 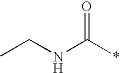 |
| 8) | 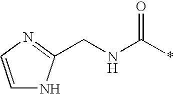 |
| 9) | 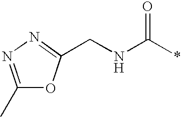 |
| 10) | 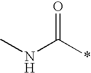 |
| 11) | 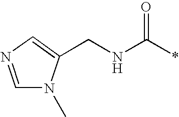 |
| 12) | 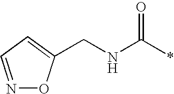 |
| 13) | 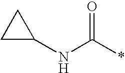 |
| 14) | 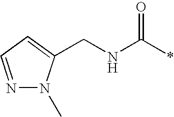 |
| 15) | 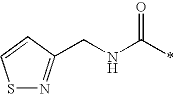 |
| 16) | 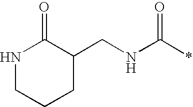 |
| 17) | 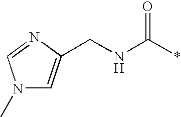 |
| 18) | 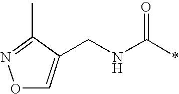 |
| 19) | 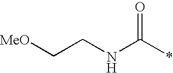 |
| 20) | 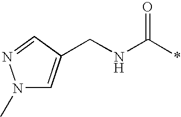 |
| 21) | 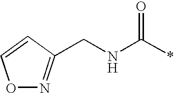 |
| 22) | 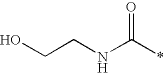 |
| 23) | 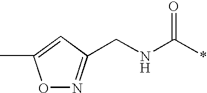 |
| 24) | 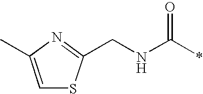 |
| 25) | 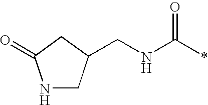 |
| 26) | 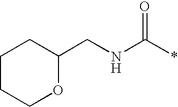 |
| 27) | 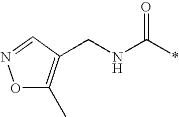 |
| 28) | 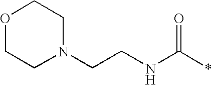 |
| 29) | 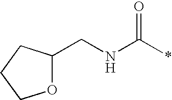 |
| 30) | 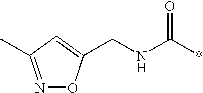 |
| 31) | 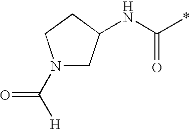 |
| 32) | 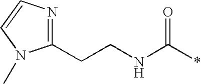 |
| 33) | 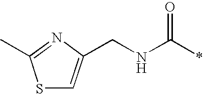 |
| 34) | 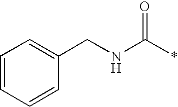 |
| 35) | 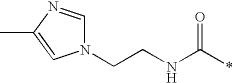 |
| 36) | 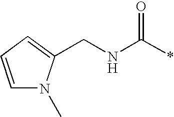 |
| 37) | 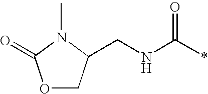 |
| 38) | 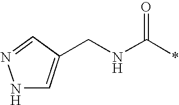 |
| 39) | 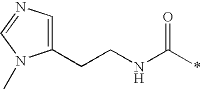 |
| 40) | 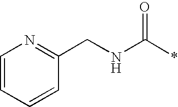 |
| 41) | 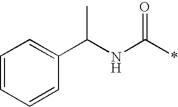 |
| 42) | 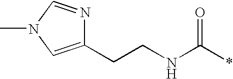 |
| 43) | 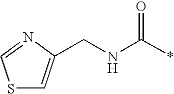 |
| 44) | 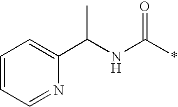 |
| 45) | 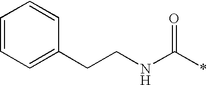 |
| 46) | 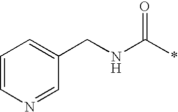 |
| 47) | 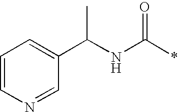 |
| 48) | 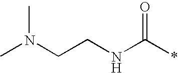 |
| 49) | 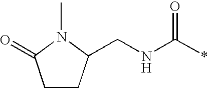 |
| 50) | 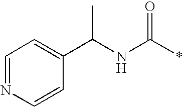 |
| 51) | 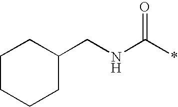 |
| 52) | 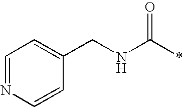 |
| 53) | 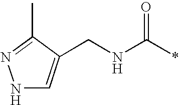 |
| 54) | 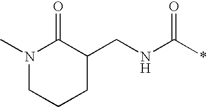 |
| 55) | 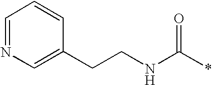 |
| 56) | 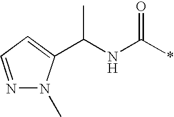 |
| 57) | 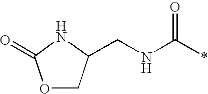 |
| 58) | 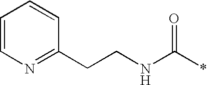 |
| 59) | 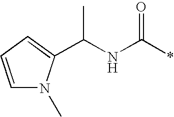 |
| 60) | 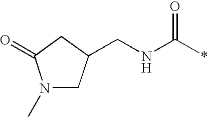 |
| 61) | 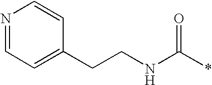 |
| 62) | 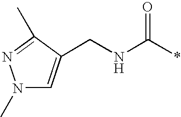 |
| 63) | 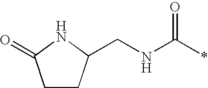 |
| 64) | 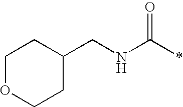 |
| 65) | 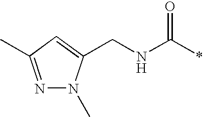 |
| 66) | 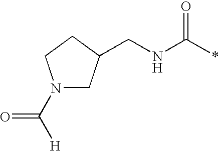 |
| 67) | 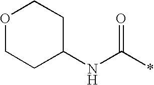 |
| 68) | 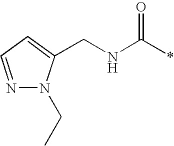 |
| 69) | 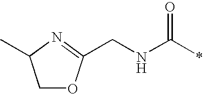 |
| 70) | 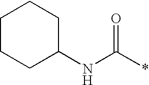 |
| 71) | 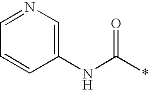 |
| 72) | 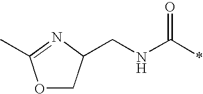 |

Compounds of formula I according to the present invention can be prepared as described below or as shown in the following reaction schemes or similarly or analogously thereto according to preparation procedures or synthesis strategies familiar to the person skilled in the art. Accordingly, compounds of formula I according to the present invention can be obtained as specified by way of example in the following examples, or similarly or analogously thereto.

Thus, as shown in reaction scheme 1 below, a compound of formula VI, in which PG is a suitable temporary protective group, such as for example tertbutoxycarbonyl (Boc) or one of those mentioned in “Protective Groups in Organic Synthesis” by T. Greene and P. Wuts (John Wiley & Sons, Inc. 1999, 3^{rd} Ed.) or in “Protecting Groups (Thieme Foundations Organic Chemistry Series N Group” by P. Kocienski (Thieme Medical Publishers, 2000), can be condensed with malonitrile in the presence of sulfur and a suitable base, such as for example an amine (e.g. diethyl amine or morpholine), to give corresponding compounds of formula V in a manner known to the person skilled in the art (e.g. according to a Gewald reaction) or as described in the following examples.

Compounds of formula VI are known or can be obtained in an art-known manner.

Compounds of formula V can be acylated with compounds of formula Rb—C(O)—X, in which Rb has the meanings mentioned above and X is a suitable leaving group, preferably a chlorine atom, in an acylation reaction under conditions habitual per se. Subsequent deprotection of the protective group PG in a manner customary per se for the skilled person gives compounds of formula IV, in which Rb has the meanings as mentioned above.

Alternatively, compounds of the formula IV can also be prepared from the corresponding compounds of formula V and corresponding compounds of formula Rb—C(O)—X, in which X is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide, diisopropylcarbodiimide or, preferably, 1-ethyl-3-(3-dimethyl-aminopropyl)carbodiimide hydrochloride (EDC)), azodicarboxylic acid derivatives (e.g. diethyl azodi-carboxylate), uronium salts [e.g. O-(benzotriazol-1-yl)-N,N,N′,N′-tetramethyluronium tetrafluoroborate or O-(benzotriazol-1yl)-N,N,N′,N′-tetramthyl-uronium-hexafluorophosphate] and N,N′-carbonyldiimidazole. Optionally, this amide bond formation may be obtained under microwave assistance. Subsequent removal of the temporary protecting group PG gives the desired compounds of formula IV.

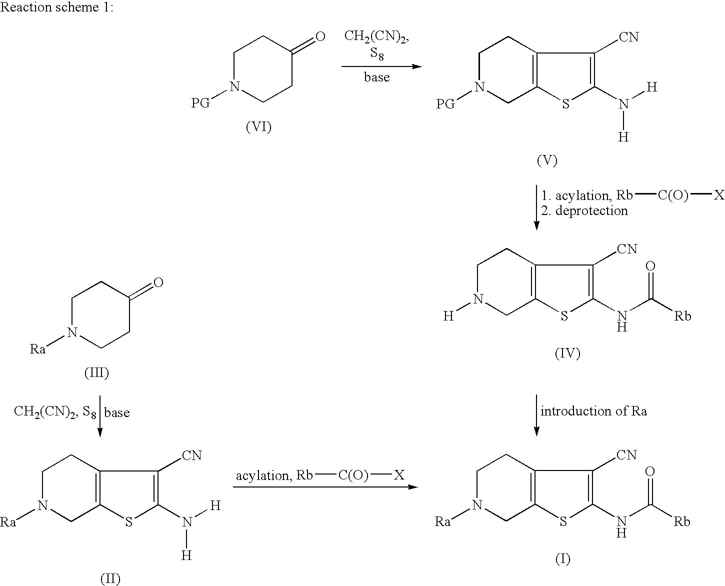

Compounds of formula IV can be converted into desired compounds of formula I by introduction of the group Ra via urea formation reaction. This urea formation reaction can be carried out in a manner as described afore, or analogously to the methods known to the person skilled in the art, or as described by way of example in the following examples. The appropriate starting compounds for this urea formation reaction are art-known or can be obtained according to art-known procedures or analogously or similarly as disclosed for known compounds.

Thus, in more detail, compounds of formula I are obtained from compounds of formula IV by reacting in a first step compounds of formula IV with phosgene or phosgene equivalents, e.g. carbonyl diimidazole, under basic conditions (e.g. an excess of amine of formula IV) in aprotic solvents (e.g. dichloro-methane). In a second step, the resulting activated intermediate is treated with the corresponding amine of formula R1-YH, in which Y is NR11 and R1 and R11 have the meanings mentioned above, in presence of a suitable base (e.g. triethylamine) in aprotic solvents.

In an alternative synthesis route, compounds of formula III, in which Ra has the meanings given above, may be condensed with malonitrile in the presence of sulfur and a suitable base as described above to give corresponding compounds of formula II.

Compounds of formula II can be reacted with compounds of formula Rb—C(O)—X in an acylation reaction analogously as described above to give the desired compounds of formula I, in which Ra and Rb have the meanings given above.

Compounds of formula III are known or can be obtained according to known procedures, or analogously or similarly thereto.

Acid derivatives of formula Rb—C(O)—X are known, commercially available or can be prepared as it is known for the skilled person, e.g. from the corresponding carboxylic acids.

Carboxylic acids of formula Rb—C(O)—OH are known, commercially available or can be obtained as it is habitual for the skilled person, e.g. analogously or similarly to standard procedures.

Thus, for example, carboxylic acids of formula Rb—C(O)—OH, in which Rb is —CH₂—CH₂-Q or —CH₂—CH(CH₃)-Q, in which Q has the meanings given above, can be obtained via CC-coupling reactions, such as e.g. by Heck or Knoevenagel reaction or, in particular, starting from aldehydes of the formula Q-CHO or ketones, especially methylketones, of the formula Q-C(O)CH₃, respectively, by Horner-Wadsworth-Emmons reaction, and then hydration reaction and, if necessary, hydrolysis of the corresponding esters obtained.

β-Methyl-propionic acids can be also obtained as given in J. Org. Chem. 61, 16, 1996, 5510-5516 and Tetrahedron Lett. 37, 10, 1996, 1683-1686 and subsequent hydration, such as e.g. described in the following examples, or analogously or similarly thereto.

In this context, there are several options for the synthesis of enantiomerically pure β-methyl-propionic acids known in literature, e.g.:
asymmetric addition of phenylboronic acids to α-,β-unsaturated esters using chiral catalysts (see e.g. S. Sakuma, M. Sakai, R. Itooka, N. Miyaura J. Org. Chem. 2000, 65, 5951-5955),
asymmetric Michael addition to α-,β-unsaturated esters using chiral auxiliaries (see e.g. J. Ezquerra, L. Prieto, C. Avendano, J. L. Martos, E. dela Cuesta, Tetrahedr. Lett. 1999, 40, 1575-1578),
asymmetric hydrogenation of α-,β-unsaturated esters and acids (see e.g. T. Uemura, X. Zhang, K. Matsumura, et al., J. Org. Chem. 1996, 61, 5510-5516; or W. Tang, W. Wang, X. Zhang Angew. Chem. Int. Ed 2003, 42(8), 943-946); or F. Menges, A. Pfaltz, Adv. Synth. Catal. 2002, 344, 40-44, or
asymmetric hydrosilylation of α-,β-unsaturated esters (see e.g. B. Lipshutz, J. M. Servesko, B. R. Taft: J. Am. Chem. Soc. 2004, 126(27), 8352-8353).

For more specific example of preparation of propionic or butyric acids of formula Rb—C(O)—OH, 3-(2-methoxyphenyl)propanoic acid is described e.g. in U.S. Pat. No. 4,567,053 or in J. Org. Chem. 69, 11, 2004, 3610-3619; 3-(3-methoxyphenyl)propanoic acid is described e.g. in J. Heterocycl. Chem. 26, 1989, 365-369; 3-(2-ethoxyphenyl)propanoic acid is described e.g. in Justus Liebigs Ann. Chem., 226, 1884, 351; 3-(3-ethoxyphenyl)propanoic acid is described e.g. in Justus Liebigs Ann. Chem. 736, 1970, 110-125; 3-(2-methoxy-phenyl)-butyric acid is described e.g. in J. Am. Chem. Soc., 61, 1939, 3039; and 3-(3-methoxy-phenyl)-butyric acid is described e.g. in J. Chem. Soc. Perkin Trans. 1, 1972, 1186, 1190.

Further on, for example, carboxylic acids of formula Rb—C(O)—OH, in which Rb is -T-Q, in which T is 1,2-cyclopropylene and Q has the meanings given above, can be obtained, starting from aldehydes of the formula Q-CHO, via Knoevenagel or Horner-Wadsworth-Emmons reaction, and then cyclopropanation reaction of the double bond (e.g. by Simmons-Smith reaction or, in particular, by Corey-Chaykovsky cyclopropanation reaction using dimethylsulfoxonium methylide) and, if necessary, hydrolysis of the corresponding esters obtained.

In this context, there are several options for asymmetric cyclopropanation known in literature, which may be used for the synthesis of enantiomerically pure cyclopropanecarboxylic acids, e.g.:
asymmetric addition of a metal (e.g. Cu, Rh, Ru, Co) carbene or carbenoid complex to an alkene (see e.g. Organic Letters 2004 Vol. 6, No. 5, 855-857),
catalytic asymmetric cyclopropanation using diazomethane or a derivative thereof and a chiral transition metal (e.g. Cu) complex (see e.g. Tetrahedron Asymmetry 2003, 14, 867-872),
asymmetric Simmons-Smith cyclopropanation, or
asymmetric Michael-initiated ring closure (MIRC) using ylides, e.g. reaction of chiral sulfonium ylides with acrylic acid derivatives (see e.g. Synlett 2005, 10, 1621-1623).

Aldehydes of formula Q-CHO and methylketones of formula Q-C(O)CH₃, in which Q has the meanings given above, are known or can be obtained in a manner customary for the skilled person analogously or similarly to known compounds.

The amino building blocks of formula R1-YH, in which Y is NR11 and R1 and R11 have the meanings given above, are known or can be obtained according to known procedures or as described herein, or analogously or similarly thereto.

Thus e.g. these amines can be obtained from the corresponding alcohols via activation of the hydroxyl radical with a suitable leaving group (e.g. Ms, Ts, Br, Cl or the like), nucleophilic substitution with an amine or azide and, in the case of azide, reduction of the azido group to obtain primary amines. Primary amines can be converted into secondary amines as it is habitual for the skilled person (e.g. by reductive amination reaction). Alternatively, amines can be obtained from the corresponding aldehydes or ketones by reductive amination reaction. Yet alternatively, amines or azides can be obtained by nucleophilic substitution reaction from the corresponding halo-alkyl compounds, which can be prepared from the corresponding alcohols as mentioned afore or from the corresponding alkyl compounds (e.g. HarB-alkyl compounds) by halogenation reaction (e.g. chlorination or bromination).

The alcohol building blocks of formula R1-YH, in which Y is O and R1 has the meanings given above, are known or can be obtained according to known procedures or as described herein, or analogously or similarly thereto.

Thus, for example, alcohol building blocks can be obtained from the corresponding aldehydes, carboxylic acids or carboxylic acid esters (which are known or which can be obtained according to known procedures) by standard reduction reactions.

When HarB-substituted alcohols, in which HarB has the meanings given above, are used as building blocks, these alcohols can be also obtained via CC-coupling reaction or nucleophilic substitution reaction of appropriate building blocks. Thus, e.g. HarB-CH₂—OH or HarB-CH₂—CH₂—OH, respectively, can be obtained from the corresponding heteroaromatic compounds by hydroxymethylation (e.g. metallation/reaction with formaldehyde or the like) or hydroxyethylation (e.g. metallation/reaction with ethylene oxide or the like), respectively.

Compounds of formula HarB-CH₂—OH or HarB-C(CH₃)H—OH, in which HarB is attached via a ring carbon atom to the methylene or ethylidene moiety, respectively, and has the meanings given above (e.g. substituted or unsubstituted pyridyl, 1N-methyl-imidazolyl or the like), can be obtained from the corresponding aldehydes (or acids or acid esters) or ketones of the formula HarB-CHO (or HarB-CO₂R) or HarB-C(O)CH₃, respectively, by art-known reduction reaction.

Aldehydes of the formula HarB-CHO are known or can be obtained as it is known for the skilled person, such as e.g. from the corresponding heteroaromatic compounds by formylation reaction or from the corresponding methyl-substituted derivatives of formula HarB-CH₃ by oxidation reaction.

Some aldehydes can be obtained as described e.g. for 4-methoxy-pyridin-2-carbaldehyde in Ashimori et al, Chem Pharm Bull 38, 2446-2458 (1990) or analogously or similarly thereto.

Compounds of formula HarB-CH₂—CH₂—NH₂, in which HarB is attached via a ring carbon atom to the ethylene moiety and has the meanings given above (e.g. substituted or unsubstituted pyridyl, 1N-methyl-imidazolyl or the like), can be obtained by CC-coupling reaction such as e.g. starting from aldehydes of the formula HarB-CHO by nitro aldol condensation and then hydrogenation (reduction) of the double bond and the nitro group, or starting from the corresponding compounds of formula HarB-CH₂—X, in which X is a suitable leaving group (such as e.g. OMs, OTs, Br, Cl or the like), by nucleophilic substitution with cyanide and then reduction of the cyano group.

Compounds of formula HetB-CH₂—CH₂—NH₂ or HarB-CH₂—CH₂—NH₂, in which HetB and HarB are attached via a ring nitrogen atom to the ethylene moiety and have the meanings given above (e.g. azol-1-yl such as imidazol-1-yl or the like), can be obtained by nucleophilic substitution reaction of corresponding compounds of formula HetB or HarB (e.g. azoles), respectively, with compounds of formula X—CH₂—CH₂—NH₂, in which X is a suitable leaving group, such as e.g. chlorine or bromine, (if necessary, the free amino group can be protected by a temporary protecting group), or, in the case of HarB, with α-halo-carboxamides of the formula X—CH₂—C(O)NH₂ (X is Cl or Br) or the like to give compounds of formula HarB-CH₂—C(O)NH₂, which are reduced to obtain compounds of formula HarB-CH₂—CH₂—NH₂.

In more detail, some of the amino- or alcohol building blocks of formula R1-YH, in which Y is O or NR11 and R1 and R11 have the meanings given above, can be purchased from one or more of the following companies: Sigma-Aldrich, Acros Organics, Fluorochem Ltd, ABCR GmbH KG, Maybridge plc, Apollo Scientific Ltd, ASDI Inc., Anichem LLC, MicroChemistry Ltd, Rare Chemicals GmbH, J & W PharmLab LLC, Oakwood Products Inc, Ambinter SARL, Aurora Fine Chemicals, Matrix Scientific, AKos Consulting and Solutions GmbH, Interchim, ChemPacific, Beta Pharma Inc., Wako Pure Chemicals Industries Ltd, Chemstep and Lancaster Synthesis Ltd.

Alternatively, the amino- or alcohol building blocks of formula R1-YH, in which Y is O or NR11 and R1 and R11 have the meanings given above, can be synthesized by methods known in the literature, or analogously or similarly thereto. Some methods are mentioned in “Science of Synthesis: Houben-Weyl methods of molecular transformations”, Eds. D. Bellus et al. (Thieme, 2002). As examples, the following building blocks may be synthesized by processes that are published in the indicated literature: 5-isoxazolyl-methylamine (D. G. Barrett et al., Bioorg. & Med. Chem. Lett. 2004, 14, 2543-2546), muscimol (P. Pevarello, M. Varasi, Synth. Commun. 1992, 22, 1939-48), (5-methyl-4-isoxazolyl)-methylamine, (3-methyl-4-isoxazolyl)-methylamine (M. Yamada et al., JP 03246283 A2 19911101 (1991)), 2-thiazolylmethylamine (A. Dondoni et al., Synthesis 1996, 641-646), (1-methyl-1H-imidazol-2-yl)-methylamine (S. Price et al., Tetrahedron Lett. 2004, 45, 5581-5583), 4-aminomethyltetrahydropyran (S, Nishino et al., WO 2005028410 A1 20050331 (2005)), 4-aminotetrahydropyran (M. Allegretti et al., J. Med. Chem. 2005, 48, 4312-4331), 4-aminomethyltetrahydropyran (R. Partch, Tetrahedron Lett. 1966, 1361-4), 3-aminotetrahydropyran (F. Alcudia et al., Anales de Quimica, Serie C: Quimica Organica y Bioquimica 1988, 84, 148-55), 2-imidazol-1-yl-ethylamine (W. B. Wright Jr. et al., J. Med. Chem. 1986, 29, 523-30), 3-aminomethylisothiazole or 4-aminomethyl-3-methylisothiazole (U.S. Pat. No. 3,838,161), 2-amino-4,5-dihydro-oxazoles (A. Gissibl et al., Org. Lett. 2005, 7, 2325-2328).

Yet alternatively, selected amino- or alcohol building blocks of formula HarB-(CH₂)ₘ₊₁—YH, in which Y is O or NH and HarB is bonded to the parent molecular group via a ring carbon atom and has the meanings given above and m is 0 or 1, may be synthesized by methods outlined in reaction scheme 2, or analogously or similarly thereto.

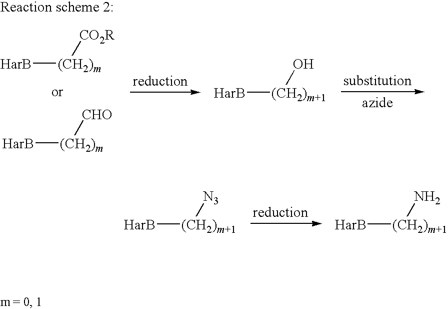

In reaction scheme 2, the carboxylic acids or carboxylic acid esters (particularly the methyl or ethyl esters) of formula HarB-(CH₂)ₘ—CO₂R (which are commercially available or are accessible by standard heterocyclic chemistry or as described herein) are reduced to the corresponding alcohols of formula HarB-(CH₂)ₘ₊₁—OH using standard reducing agents, e.g. lithium aluminium hydride. The alcohols of formula HarB-(CH₂)ₘ₊₁—OH can be transformed into the azide of formula HarB-(CH₂)ₘ₊₁—N₃ by activation of the hydroxyl group followed by substitution of azide. The activation can be achieved using a sulfonyl chloride (e.g. mesyl chloride) in combination with a base (e.g. triethyl amine) or by halogenation using an appropriate halogenation agent (e.g. sulfuryl chloride). The azide substitution can be achieved using an azide salt, e.g. sodium azide. Alternatively, the alcohols can be converted into the azides using a phosphoryl azide (e.g. diphenylphosphoryl azide) in the presence of a strong base (e.g. 1,8-diazabicyclo[5.4.0]undec-7-ene). The latter method is preferred. Finally, amines of formula HarB-(CH₂)ₘ₊₁—NH₂ can be accessed by reduction of the corresponding azides using, for example, hydrogen and catalytic amounts of palladium on charcoal. Following the above described methodology, the following building blocks may be synthesized: (5-methyl-4-isoxazolyl)-methanol, (3-methyl-4-isoxazolyl)-methanol, (5-methyl-3-isoxazolyl)-methanol, (1-methyl-1H-imidazol-5-yl)-methanol, (2,4-dimethyl-thiazol-5-yl)-methanol, (5-methyl-4-isoxazolyl)-methylamine, (3-methyl-4-isoxazolyl)-methylamine, (5-methyl-3-isoxazolyl)-methylamine, (1-methyl-1H-imidazol-5-yl)-methylamine, (2,4-dimethyl-thiazol-5-yl)-methylamine.

The alcohols of formula HarB-(CH₂)ₘ₊₁—OH, which are then further transformed as described above, may be also obtained from the corresponding aldehydes of formula HarB-(CH₂)ₘ—CHO using an appropriate reducing agent, preferably sodium borohydride or lithium aluminium hydride. The aldehydes of formula HarB-CHO can be obtained from the corresponding heterocyclic compounds of formula HarB by formylation reaction under standard formylation conditions, e.g. treatment with strong base, e.g. n-butyl lithium, followed by addition of dimethylformamide or treatment with phosphoryl chloride in the presence of dimethylformamide. Following this methodology, the following building blocks may be synthesized: (2-methyl-2H-pyrazol-3-yl)-methanol, (2-ethyl-2H-pyrazol-3-yl)-methanol, (1-methyl-1H-imidazol-2-yl)-methanol, (1-methyl-1H-pyrazol-4-yl)-methanol, (2-methyl-2H-pyrazol-3-yl)-methylamine, (2-ethyl-2H-pyrazol-3-yl)-methylamine, (1-methyl-1H-imidazol-2-yl)-methylamine, (1-methyl-1H-pyrazol-4-yl)-methylamine.

The aldehydes of formula HarB-CHO, which are then further transformed as described above, may be also obtained from the corresponding halogen compounds of formula HarB-X, in which X is chlorine, bromine or iodine, by lithium-halogen exchange. Typical reaction conditions for this transformation are treatment of this halogen compounds of formula HarB-X with t-butyl lithium at low temperature (−70° C.-80° C.), followed by addition of dimethylformamide. Following this methodology, the following building blocks may be synthesized: 2-thiazolyl-methanol, 2-thiazolyl-methylamine.

The halo-methyl compounds of formula HarB-CH₂—X, in which X is bromine or chlorine, which are then further transformed as described above, may be obtained from the corresponding methyl compounds of formula HarB-CH₃ by halogenation reaction using an appropriate halogenating agent, e.g. N-bromo-succinimide or N-chlorosuccinimide. Following this methodology, the following building blocks may be synthesized: 5-isoxazyl-methanol, 3-isoxazyl-methanol, 5-isoxazyl-methylamine, 3-isoxazyl-methylamine

Yet alternatively, selected amino building blocks of formula HarB-CH₂CH₂—NH₂, in which HarB is bonded to the parent molecular group via a ring carbon atom and has the meanings given above, may be synthesized by methods outlined in reaction scheme 3, or analogously or similarly thereto.

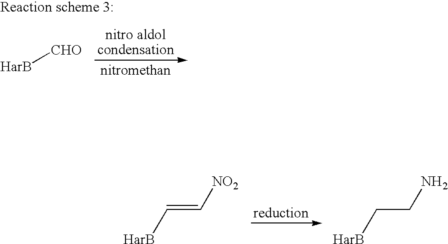

Yet alternatively, selected amino building blocks of formula HarB-CH₂CH₂—NH₂, in which HarB is bonded to the parent molecular group via a ring carbon atom and has the meanings given above, may be synthesized by methods outlined in reaction scheme 4, or analogously or similarly thereto.

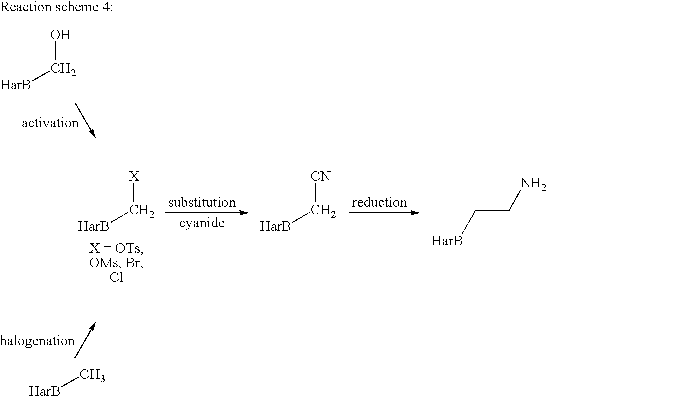

Yet alternatively, selected amino- or alcohol building blocks of formula HarB-C(CH₃)H—YH, in which Y is O or NH and HarB is bonded to the parent molecular group via a ring carbon atom and has the meanings given above, may be synthesized by methods outlined in reaction scheme 5, or analogously or similarly thereto.

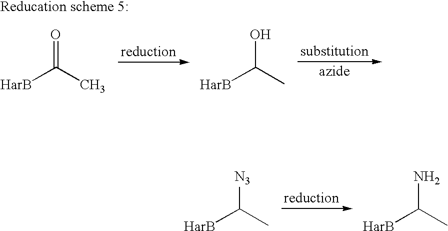

Yet alternatively, selected amino building blocks of formula HarB-CH₂CH₂—NH₂, in which HarB is bonded to the parent molecular group via a ring nitrogen atom and has the meanings given above, may be synthesized by methods outlined in reaction scheme 6, or analogously or similarly thereto.

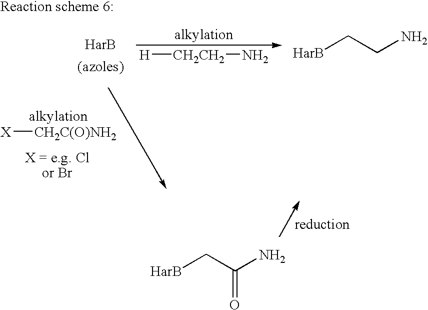

In reaction scheme 6, compounds of formula HarB (e.g. azoles), which have an alkylatable nitrogen (NH) atom, can be reacted with α-halo-carboxamides of formula X—CH₂C(O)NH₂, in which X is chlorine or bromine, (e.g. 2-bromoacetamide) in the presence of an appropriate base (e.g. sodium hydride) to give rise to corresponding compounds of formula HarB-CH₂C(O)NH₂. The amides of formula HarB-CH₂C(O)NH₂ can be reduced to the corresponding amines of formula HarB-CH₂CH₂—NH₂ using an appropriate reducing agent, e.g. lithium aluminium hydride. Alternatively, precursors of formula HarB can be transformed directly to amines of formula HarB-CH₂CH₂—NH₂ by reaction with compounds of formula X—CH₂CH₂—NH₂, in which X is a suitable leaving group (e.g. Cl or Br), e.g. 2-chloroethylamine, under basic conditions (if necessary, the free amino group can be protected by a temporary protecting group). Following this methodology, the following building blocks can be synthesized: 2-imidazol-1-yl-ethylamine, 2-(4-methyl-imidazol-1-yl)-ethylamine.

Yet alternatively, selected amino building blocks of formula HarA-NH₂, in which HarA has the meanings given above, may be synthesized from the corresponding alcohols of formula HarA-OH by substitution with azide and then reduction of the azide to the amine.

Abovementioned precursors and compounds of formula HarB, HarB-CH₃, HarB-(CH₂)ₘ—CO₂R, HarB-C(O)CH₃, HarB-(CH₂)ₘ—CHO, HarB-X or HarA-OH are known, commercially available or can be obtained according to known procedures, e.g. by standard heterocyclic chemistry.

Yet alternatively, selected alcohol building blocks of formula HetA-OH or HetB-(CH₂)ₘ—OH, in which HetA and HetB are 1N-(1-4C-alkylcarbonyl)-piperidinyl, 1N-(1-4C-alkylcarbonyl)-pyrrolidinyl, 1N-(formyl)-piperidinyl or 1N-(formyl)-pyrrolidinyl and m is 1 or 2, may be obtained from the correspondding cyclic NH-amines of formula HetA-OH or HetB-(CH₂)ₘ—OH, in which HetA and HetB are 1N—(H)-piperidinyl or 1N—(H)-pyrrolidinyl, (which cyclic NH-amines are known or can be obtained according to known procedures), by standard N-acylation reactions.

Yet alternatively, selected alcohol building blocks of formula HetA-OH or HetC-(CH₂)ₘ—OH, in which HetA and HetC are 1N-(1-4C-alkyl)-piperidin-2-onyl, 1N-(1-4C-alkyl)-pyrrolidin-2-onyl, 3N-(1-4C-alkyl)-oxazolidin-2-onyl, 1N-(1-4C-alkyl)-3N-(1-4C-alkyl)-imidazolidin-2-onyl or 1N—(H)-3N-(1-4C-alkyl)-imidazolidin-2-onyl and m is 1 or 2, may be obtained from the corresponding cyclic NH-amides of formula HetA-OH or HetB-(CH₂)ₘ—OH, in which HetA and HetC are 1N—(H)-piperidin-2-onyl, 1N—(H)-pyrrolidin-2-onyl, 3N—(H)-oxazolidin-2-onyl or 1N—(H)-3N—(H)-imidazolidin-2-onyl, (which cyclic NH-amides are known or can be obtained according to known procedures), by standard N-alkylation reactions (if necessary, the free hydroxyl group can be protected by a suitable temporary protecting group during this N-alkylation reaction).

Cyclic NH-amides of formula HetA-OH or HetC-(CH₂)ₘ—OH, in which HetA and HetC are 1N—(H)-piperidin-2-onyl, 1N—(H)-pyrrolidin-2-onyl, 3N—(H)-oxazolidin-2-onyl or 1N—(H)-3N—(H)-imidazolidin-2-onyl and m is 0 or 1, may be be prepared as described e.g. in K. J. Lidstrom et al. Synth. Commun. 1990, 20, 2335-2337; S. Klutchko et al. J. Med. Chem. 1981, 24, 104-109; N. I. Carruthers et al. J. Chem. Res., Synopses 1996, 430-431; M. P. Sibi et al. Synlett 2004, 1211-1214; S. Hanessian et al. J. Org. Chem. 1993, 58, 5032-5034; A. Otto et al. Tetrahedron: Asymmetry 1999, 10, 3381-3389; or R. Fischer, EP 537606 (U.S. Pat. No. 5,286,875) A119930421 (1993), or analogously or similarly thereto.

Yet alternatively, selected alcohol building blocks of formula HarB-CH₂—OH, in which HarB is optionally substituted by R13, and is 4,5-dihydro-oxazol-4-yl, in which R13 has the meanings given above (in particular R13 is 1-4C-alkyl, in more particular methyl) may be obtained as outlined in reaction scheme 7 starting from corresponding 2-acylamino-propane-1,3-diol compounds, particularly 2-acetylamino-propane-1,3-diole (which diole compounds can be prepared analogously to W. Zimmermann, Archiv der Pharmazie (Weinheim, Germany), 1989, 322, 639-640), via cyclization reaction, for example via Wipf cyclodehydration using Burgess reagent e.g. as described in P. Wipf et al. Org. Lett. 2006, 8, 2381-2384.

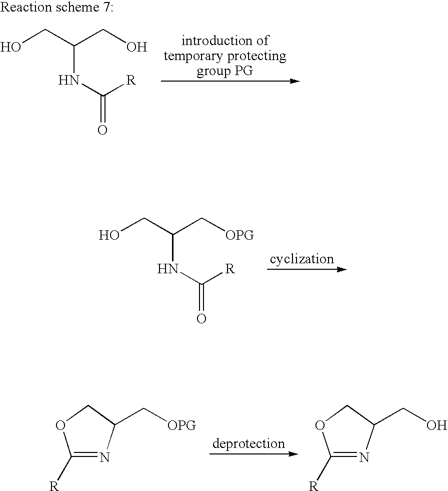

R is H or R13, in particular R13 is 1-4C-alkyl, in more particular R13 is methyl

Yet alternatively, selected alcohol building blocks of formula HarB-CH₂—OH, in which HarB is optionally substituted by R13, and is 4,5-dihydro-oxazol-2-yl, in which R13 has the meanings given above (in particular R13 is 1-4C-alkyl, in more particular methyl) may be obtained as outlined in reaction scheme 8 starting from corresponding aminoalcohol compounds, particularly 2-amino-propanol, via cyclization with glycolic acid derivatives (in which the hydroxy function is protected with a suitable temporary protecting group) suitably in the presence of an appropriate (Lewis) acid catalyst, for example in a manner as described in L. N. Pridgen et al. J. Heterocycl. Chem. 1983, 20, 1223, or in J. V. Allen et al. Tetrahedron Asymmetry 1994, 5, 277-282, or in W. E. Fristad et al. EP 394849 A1 19901031 (1990), or by azeotropic removal of water as described in P. Stepnicka et al. Collect. Czech. Chem. Commun. 2003, 68, 1206-1232.

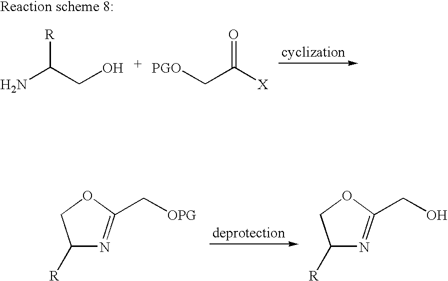

R is H or R13, in particular R13 is 1-4C-alkyl, in more particular R13 is methyl
X is Cl or OMe
PG is a suitable temporary protecting group, e.g. allyl

Yet alternatively, selected alcohol building blocks of formula HarB-CH₂CH₂—OH, in which HarB is optionally substituted by R13, and is 4,5-dihydro-oxazol-2-yl, in which R13 has the meanings given above (in particular R13 is 1-4C-alkyl, in more particular methyl) may be obtained starting from corresponding 2-methyl-4,5-dihydro-oxazoles of formula HarB-CH₃ (which 2-methyl-4,5-dihydro-oxazoles are known or can be obtained according to known procedures or analogously as described above), via hydroxymethylation reaction using e.g. formaldehyde in the presence of a base, for example as described in W. Seeliger et al. Angew. Chem. 1966, 78, 913-27.

The aforementioned alcohol building blocks can be converted into the corresponding amino building blocks such as e.g. described above.

It is to be understood for the skilled worker, that certain compounds of this invention can be converted into further compounds of this invention by art-known synthesis strategies and reactions habitual per se to a person of ordinary skill in the art.

Therefore, optionally, compounds of formula I can be converted into further compounds of formula I by methods known to one of ordinary skill in the art. More specifically, for example, from compounds of the formula I in which
a) Raa is acyloxy, such as e.g. acetoxy, the corresponding free hydroxyl compounds can be obtained by removal of the acyl group, such as e.g. by saponification reaction;
b) Rab and Rac taken together form a cyclic acetal or ketal, such as e.g. the 2,2-dimethyl-[1,3]dioxolan acetal, the corresponding free dihydroxy compounds can be obtained by cleavage of the acetal or ketal, such as e.g. by deacetalization reaction;
c) Raa is an ester group, such as e.g. methoxycarbonyl, the corresponding free carboxyl compounds can be obtained by deesterification, such as e.g. by saponification reaction.

The methods mentioned under a) to c) can be expediently carried out analogously to the methods known to the person skilled in the art or as described by way of example in the following examples.

Optionally, compounds of the formula I can be converted into their salts, or, optionally, salts of the compounds of the formula I can be converted into the free compounds. Corresponding processes are habitual per se to the skilled person.

When one of the final steps or purification is carried out under the presence of an inorganic or organic acid (e.g. hydrochloric, trifluoroacetic, acetic or formic acid or the like), the compounds of formula I may be obtained—depending on their individual chemical nature and the individual nature of the acid used—as free base or containing said acid in an stoechiometric or non-stoechiometric quantity. The amount of the acid contained can be determined according to art-known procedures, e.g. by titration or NMR.

It is moreover known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in “Protective Groups in Organic Synthesis” by T. Greene and P. Wuts (John Wiley & Sons, Inc. 1999, 3^{rd} Ed.) or in “Protecting Groups (Thieme Foundations Organic Chemistry Series N Group” by P. Kocienski (Thieme Medical Publishers, 2000).

The substances according to the invention are isolated and purified in a manner known per se, for example by distilling off the solvent under reduced pressure and recrystallizing the residue obtained from a suitable solvent or subjecting it to one of the customary purification methods, such as, for example, column chromatography on a suitable support material.

Salts are obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low-molecular-weight aliphatic alcohol, such as methanol, ethanol or isopropanol) which contains the desired acid or base, or to which the desired acid or base is then added. The salts are obtained by filtering, reprecipitating, precipitating with a nonsolvent for the addition salt or by evaporating the solvent. Salts obtained can be converted into the free compounds, which can in turn be converted into salts, by alkalization or by acidification. In this manner, pharmacologically unacceptable salts can be converted into pharmacologically acceptable salts.

Suitably, the conversions mentioned in this invention can be carried out analogously or similarly to methods which are familiar per se to the person skilled in the art.

The person skilled in the art may be familiar on the basis of his/her knowledge and on the basis of those synthesis routes, which are shown and described within the description of this invention, to find other possible synthesis routes for compounds according to this invention. All these other possible synthesis routes are also part of this invention.

The present invention also relates to the intermediates (including their salts, stereoisomers and salts of the stereoisomers), methods and processes, which are disclosed herein and which are useful in synthesizing compounds according to this invention. Thus, the present invention also relates to processes disclosed herein for preparing compounds according to this invention, which processes comprise one or more steps of converting and/or reacting the mentioned intermediates with the appropriate reaction partners under conditions as disclosed herein.

Having described the invention in detail, the scope of the present invention is not limited only to those described characteristics or embodiments. As will be apparent to persons skilled in the art, modifications, analogies, variations, derivations, homologisations, alternatives and adaptations to the described invention can be made on the base of art-known knowledge and/or, particularly, on the base of the disclosure (e.g. the explicite, implicite or inherent disclosure) of the present invention without departing from the spirit and scope of this invention as defined by the scope of the appended claims.

The following examples serve to illustrate the invention further without restricting it. Likewise, further compounds according to this invention, the preparation of which is not explicitly described, can be prepared in an analogous or similar manner or in a manner familiar per se to the person skilled in the art using customary process techniques.

Any or all of the compounds of formula I according to the present invention which are mentioned in the following examples as final compounds as well as their salts, stereoisomers and salts of the stereoisomers are a preferred subject of the present invention.

In the examples, MS stands for mass spectrum, M is the molecular ion in mass spectroscopy, calc. for calculated, fnd. for found, Boc for the tertbutoxycarbonyl group, EDC or EDCI for 1-ethyl-3-(3-dimethyl-aminopropyl)carbodiimide hydrochloride and other abbreviations have their meanings customary per se to the skilled person.

Further on, according to common practice in stereochemistry, the term “(RS)” characterizes a racemate comprising the one enantiomer having the configuration R and the other enantiomer having the configuration S; each of these enantiomers and their salts in pure form as well as their mixtures including the racemic mixtures is part of this invention.

Yet further on, according to common practice in stereochemistry, when more than one chiral center is present in a molecule, the symbols RS and SR are used to denote the specific configuration of each of the chiral centers of a racemate. In more detail, for example, the term “(1RS,2RS)” stands for a racemate (racemic mixture) comprising the one enantiomer having the configuration (1R,2R) and the other enantiomer having the configuration (1S,2S); each of these enantiomers and their salts in pure form as well as their mixtures including the racemic mixtures is part of this invention.

### EXAMPLES

### Final Compounds

Compound names throughout this document have been generated by use of AutoNom Engine, version 4.0, by Beilstein Institut, Frankfurt, Germany.

### General Procedure A:

Using the appropriate starting materials A1-A24 and the appropriate amines, the final compounds can be prepared as described as follows:

1.8 eq. of the appropriate amine is dissolved in dichloromethane and 1.25 eq. of CDI is added. After the gas evolution has deceased 1 eq. of the appropriate starting material A1 to A24 together with 5 eq. triethylamine are added. After stirring over night the reaction mixture is concentrated and the desired product is obtained after crystallization or is triturated from ethanol. In case crystallization or trituaration does not give sufficiently pure product, the evaporated reaction mixture or impure product is subjected to flash chromatography or HPLC with or without subsequent cystallization.

Using this procedure the following compounds may be prepared:
1. 3-Cyano-2-[3-(2-methoxy-phenyl)-butanoylamino]-4,7-dihydro-5H-thieno[2,3-c]pyridine-6-carboxylic acid(pyridin-2-ylmethyl)-amide

MS: calc.: C, 26; H, 27; N, 5; O, 3; S, (489.60). fnd.: 490.1; [M+H].
2. 3-Cyano-2-[3-(2-ethoxy-phenyl)-butanoylamino]-4,7-dihydro-5H-thieno[2,3-c]pyridine-6-carboxylic acid(pyridin-2-ylmethyl)-amide

MS: calc.: C, 27; H, 29; N, 5; O, 3; S, (503.63). fnd.: 504.1; [M+H].
3. 3-Cyano-2-[3-(3-methoxy-phenyl)-propanoylamino]-4,7-dihydro-5H-thieno[2,3-c]pyridine-6-carboxylic acid(pyridin-2-ylmethyl)-amide

MS: calc.: C, 25; H, 25; N, 5; O, 3; S, (475.57). fnd.: 476.1; [M+H].
4. 3-Cyano-2-[3-(2-methoxy-phenyl)-propanoylamino]-4,7-dihydro-5H-thieno[2,3-c]pyridine-6-carboxylic acid(pyridin-2-ylmethyl)-amide

MS: calc.: C, 25; H, 25; N, 5; O, 3; S, (475.57). fnd.: 476.1; [M+H].
5. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(pyridin-2-ylmethyl)-amide

MS: calc.: C, 26; H, 25; N, 5; O, 3; S, (487.58). fnd.: 488.0; [M+H].
6. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(pyridin-2-ylmethyl)-amide

MS: calc.: C, 26; H, 25; N, 5; O, 3; S, (487.58). fnd.: 488.0; [M+H].
7. 3-Cyano-2-((RS)-3-phenyl-butanoylamino)-4,7-dihydro-5H-thieno[2,3-c]pyridine-6-carboxylic acid(pyridin-2-ylmethyl)-amide

MS: calc.: C, 25; H, 25; N, 5; O, 2; S, (459.57). fnd.: 460.1; [M+H].
8. 3-Cyano-2-(3-phenyl-propanoylamino)-4,7-dihydro-5H-thieno[2,3-c]pyridine-6-carboxylic acid(pyridin-2-ylmethyl)-amide

MS: calc.: C, 24; H, 23; N, 5; O, 2; S, (445.55)
9. 3-Cyano-2-(3-cyclohexyl-propanoylamino)-4,7-dihydro-5H-thieno[2,3-c]pyridine-6-carboxylic acid(pyridin-2-ylmethyl)-amide

MS: calc.: C, 24; H, 29; N, 5; O, 2; S, (451.60). fnd.: 452.2; [M+H].
10. 3-Cyano-2-[(RS)-3-(2-methoxy-phenyl)-butanoylamino]-4,7-dihydro-5H-thieno[2,3-c]pyridine-6-carboxylic acid(pyridin-4-ylmethyl)-amide

MS: calc.: C, 26; H, 27; N, 5; O, 3; S, (489.60). fnd.: 490.2; [M+H].
11. 3-Cyano-2-[(RS)-3-(2-ethoxy-phenyl)-butanoylamino]-4,7-dihydro-5H-thieno[2,3-c]pyridine-6-carboxylic acid(pyridin-4-ylmethyl)-amide

MS: calc.: C, 27; H, 29; N, 5; O, 3; S, (503.63). fnd.: 504.2; [M+H].
12. 3-Cyano-2-[3-(3-methoxy-phenyl)-propanoylamino]-4,7-dihydro-5H-thieno[2,3-c]pyridine-6-carboxylic acid(pyridin-4-ylmethyl)-amide

MS: calc.: C, 25; H, 25; N, 5; O, 3; S, (475.57). fnd.: 476.2; [M+H].
13. 3-Cyano-2-[3-(2-methoxy-phenyl)-propanoylamino]-4,7-dihydro-5H-thieno[2,3-c]pyridine-6-carboxylic acid(pyridin-4-ylmethyl)-amide

MS: calc.: C, 25; H, 25; N, 5; O, 3; S, (475.57). fnd.: 476.1; [M+H].
14. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(pyridin-4-ylmethyl)-amide

MS: calc.: C, 26; H, 25; N, 5; O, 3; S, (487.58). fnd.: 488.2; [M+H].
15. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(pyridin-4-ylmethyl)-amide

MS: calc.: C, 26; H, 25; N, 5; O, 3; S, (487.58). fnd.: 488.2; [M+H].
16. 3-Cyano-2-((RS)-3-phenyl-butanoylamino)-4,7-dihydro-5H-thieno[2,3-c]pyridine-6-carboxylic acid(pyridin-4-ylmethyl)-amide

MS: calc.: C, 25; H, 25; N, 5; O, 2; S, (459.57). fnd.: 460.2; [M+H].
17. 3-Cyano-2-(3-phenyl-propanoylamino)-4,7-dihydro-5H-thieno[2,3-c]pyridine-6-carboxylic acid(pyridin-4-ylmethyl)-amide

MS: calc.: C, 24; H, 23; N, 5; O, 2; S, (445.55)
18. 3-Cyano-2-(3-cyclohexyl-propanoylamino)-4,7-dihydro-5H-thieno[2,3-c]pyridine-6-carboxylic acid(pyridin-4-ylmethyl)-amide

MS: calc.: C, 24; H, 29; N, 5; O, 2; S, (451.60). fnd.: 452.3; [M+H].
19. 3-Cyano-2-[(RS)-3-(2-methoxy-phenyl)-butanoylamino]-4,7-dihydro-5H-thieno[2,3-c]pyridine-6-carboxylic acid(2-pyridin-2-yl-ethyl)-amide

MS: calc.: C, 27; H, 29; N, 5; O, 3; S, (503.63). fnd.: 504.1; [M+H].
20. 3-Cyano-2-[(RS)-3-(2-ethoxy-phenyl)-butanoylamino]-4,7-dihydro-5H-thieno[2,3-c]pyridine-6-carboxylic acid(2-pyridin-2-yl-ethyl)-amide

MS: calc.: C, 28; H, 31; N, 5; O, 3; S, (517.65). fnd.: 518.2; [M+H].
21. 3-Cyano-2-[3-(3-methoxy-phenyl)-propanoylamino]-4,7-dihydro-5H-thieno[2,3-c]pyridine-6-carboxylic acid(2-pyridin-2-yl-ethyl)-amide

MS: calc.: C, 26; H, 27; N, 5; O, 3; S, (489.60). fnd.: 490.1; [M+H].
22. 3-Cyano-2-[3-(2-methoxy-phenyl)-propanoylamino]-4,7-dihydro-5H-thieno[2,3-c]pyridine-6-carboxylic acid(2-pyridin-2-yl-ethyl)-amide

MS: calc.: C, 26; H, 27; N, 5; O, 3; S, (489.60). fnd.: 489.9; [M+H].
23. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(2-pyridin-2-yl-ethyl)-amide

MS: calc.: C, 27; H, 27; N, 5; O, 3; S, (501.61). fnd.: 502.1; [M+H].
24. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(2-pyridin-2-yl-ethyl)-amide

MS: calc.: C, 27; H, 27; N, 5; O, 3; S, (501.61). fnd.: 502.1; [M+H].
25. 3-Cyano-2-((RS)-3-phenyl-butanoylamino)-4,7-dihydro-5H-thieno[2,3-c]pyridine-6-carboxylic acid(2-pyridin-2-yl-ethyl)-amide

MS: calc.: C, 26; H, 27; N, 5; O, 2; S, (473.60). fnd.: 474.1; [M+H].
26. 3-Cyano-2-(3-phenyl-propanoylamino)-4,7-dihydro-5H-thieno[2,3-c]pyridine-6-carboxylic acid(2-pyridin-2-yl-ethyl)-amide

MS: calc.: C, 25; H, 25; N, 5; O, 2; S, (459.57)
27. 3-Cyano-2-(3-cyclohexyl-propanoylamino)-4,7-dihydro-5H-thieno[2,3-c]pyridine-6-carboxylic acid(2-pyridin-2-yl-ethyl)-amide

MS: calc.: C, 25; H, 31; N, 5; O, 2; S, (465.62). fnd.: 466.1; [M+H].
28. 3-Cyano-2-[(RS)-3-(2-methoxy-phenyl)-butanoylamino]-4,7-dihydro-5H-thieno[2,3-c]pyridine-6-carboxylic acid(2-pyridin-3-yl-ethyl)-amide

MS: calc.: C, 27; H, 29; N, 5; O, 3; S, (503.63). fnd.: 504.2; [M+H].
29. 3-Cyano-2-[(RS)-3-(2-ethoxy-phenyl)-butanoylamino]-4,7-dihydro-5H-thieno[2,3-c]pyridine-6-carboxylic acid(2-pyridin-3-yl-ethyl)-amide

MS: calc.: C, 28; H, 31; N, 5; O, 3; S, (517.65). fnd.: 518.2; [M+H].
30. 3-Cyano-2-[(RS)-3-(3-methoxy-phenyl)-propanoylamino]-4,7-dihydro-5H-thieno[2,3-c]pyridine-6-carboxylic acid(2-pyridin-3-yl-ethyl)-amide

MS: calc.: C, 26; H, 27; N, 5; O, 3; S, (489.60). fnd.: 490.2; [M+H].
31. 3-Cyano-2-[3-(2-methoxy-phenyl)-propanoylamino]-4,7-dihydro-5H-thieno[2,3-c]pyridine-6-carboxylic acid(2-pyridin-3-yl-ethyl)-amide

MS: calc.: C, 26; H, 27; N, 5; O, 3; S, (489.60). fnd.: 490.2; [M+H].
32. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(2-pyridin-3-yl-ethyl)-amide

MS: calc.: C, 27; H, 27; N, 5; O, 3; S, (501.61). fnd.: 502.2; [M+H].
33. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(2-pyridin-3-yl-ethyl)-amide

MS: calc.: C, 27; H, 27; N, 5; O, 3; S, (501.61). fnd.: 502.2; [M+H].
34. 3-Cyano-2-((RS)-3-phenyl-butanoylamino)-4,7-dihydro-5H-thieno[2,3-c]pyridine-6-carboxylic acid(2-pyridin-3-yl-ethyl)-amide

MS: calc.: C, 26; H, 27; N, 5; O, 2; S, (473.60). fnd.: 474.2; [M+H].
35. 3-Cyano-2-(3-phenyl-propanoylamino)-4,7-dihydro-5H-thieno[2,3-c]pyridine-6-carboxylic acid(2-pyridin-3-yl-ethyl)-amide

MS: calc.: C, 25; H, 25; N, 5; O, 2; S, (459.57)
36. 3-Cyano-2-(3-cyclohexyl-propanoylamino)-4,7-dihydro-5H-thieno[2,3-c]pyridine-6-carboxylic acid(2-pyridin-3-yl-ethyl)-amide

MS: calc.: C, 25; H, 31; N, 5; O, 2; S, (465.62). fnd.: 466.3; [M+H].
37. 3-Cyano-2-[(RS)-3-(2-methoxy-phenyl)-butanoylamino]-4,7-dihydro-5H-thieno[2,3-c]pyridine-6-carboxylic acid(2-pyridin-4-yl-ethyl)-amide

MS: calc.: C, 27; H, 29; N, 5; O, 3; S, (503.63). fnd.: 504.3; [M+H].
38. 3-Cyano-2-[(RS)-3-(2-ethoxy-phenyl)-butanoylamino]-4,7-dihydro-5H-thieno[2,3-c]pyridine-6-carboxylic acid(2-pyridin-4-yl-ethyl)-amide

MS: calc.: C, 28; H, 31; N, 5; O, 3; S, (517.65). fnd.: 518.2; [M+H].
39. 3-Cyano-2-[3-(3-methoxy-phenyl)-propanoylamino]-4,7-dihydro-5H-thieno[2,3-c]pyridine-6-carboxylic acid(2-pyridin-4-yl-ethyl)-amide

MS: calc.: C, 26; H, 27; N, 5; O, 3; S, (489.60). fnd.: 490.2; [M+H].
40. 3-Cyano-2-[3-(2-methoxy-phenyl)-propanoylamino]-4,7-dihydro-5H-thieno[2,3-c]pyridine-6-carboxylic acid(2-pyridin-4-yl-ethyl)-amide

MS: calc.: C, 26; H, 27; N, 5; O, 3; S, (489.60). fnd.: 490.0; [M+H].
41. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(2-pyridin-4-yl-ethyl)-amide

MS: calc.: C, 27; H, 27; N, 5; O, 3; S, (501.61). fnd.: 502.1; [M+H].
42. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(2-pyridin-4-yl-ethyl)-amide

MS: calc.: C, 27; H, 27; N, 5; O, 3; S, (501.61). fnd.: 502.2; [M+H].
43. 3-Cyano-2-(3-phenyl-butanoylamino)-4,7-dihydro-5H-thieno[2,3-c]pyridine-6-carboxylic acid(2-pyridin-4-yl-ethyl)-amide

MS: calc.: C, 26; H, 27; N, 5; O, 2; S, (473.60). fnd.: 474.2; [M+H].
44. 3-Cyano-2-(3-phenyl-propanoylamino)-4,7-dihydro-5H-thieno[2,3-c]pyridine-6-carboxylic acid(2-pyridin-4-yl-ethyl)-amide

MS: calc.: C, 25; H, 25; N, 5; O, 2; S, (459.57)
45. 3-Cyano-2-(3-cyclohexyl-propanoylamino)-4,7-dihydro-5H-thieno[2,3-c]pyridine-6-carboxylic acid(2-pyridin-4-yl-ethyl)-amide

MS: calc.: C, 25; H, 31; N, 5; O, 2; S, (465.62). fnd.: 466.3; [M+H].
46. 3-Cyano-2-[(RS)-3-(2-methoxy-phenyl)-butanoylamino]-4,7-dihydro-5H-thieno[2,3-c]pyridine-6-carboxylic acid(pyridin-3-ylmethyl)-amide

MS: calc.: C, 26; H, 27; N, 5; O, 3; S, (489.60). fnd.: 490.2; [M+H].
47. 3-Cyano-2-[(RS)-3-(2-ethoxy-phenyl)-butanoylamino]-4,7-dihydro-5H-thieno[2,3-c]pyridine-6-carboxylic acid(pyridin-3-ylmethyl)-amide

MS: calc.: C, 27; H, 29; N, 5; O, 3; S, (503.63). fnd.: 504.3; [M+H].
48. 3-Cyano-2-[3-(3-methoxy-phenyl)-propanoylamino]-4,7-dihydro-5H-thieno[2,3-c]pyridine-6-carboxylic acid(pyridin-3-ylmethyl)-amide

MS: calc.: C, 25; H, 25; N, 5; O, 3; S, (475.57). fnd.: 476.1; [M+H].
49. 3-Cyano-2-[3-(2-methoxy-phenyl)-propanoylamino]-4,7-dihydro-5H-thieno[2,3-c]pyridine-6-carboxylic acid(pyridin-3-ylmethyl)-amide

MS: calc.: C, 25; H, 25; N, 5; O, 3; S, (475.57). fnd.: 476.1; [M+H].
50. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(pyridin-3-ylmethyl)-amide

MS: calc.: C, 26; H, 25; N, 5; O, 3; S, (487.58). fnd.: 488.2; [M+H].
51. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(pyridin-3-ylmethyl)-amide

MS: calc.: C, 26; H, 25; N, 5; O, 3; S, (487.58). fnd.: 488.2; [M+H].
52. 3-Cyano-2-((RS)-3-phenyl-butanoylamino)-4,7-dihydro-5H-thieno[2,3-c]pyridine-6-carboxylic acid(pyridin-3-ylmethyl)-amide

MS: calc.: C, 25; H, 25; N, 5; O, 2; S, (459.57). fnd.: 460.2; [M+H].
53. 3-Cyano-2-(3-phenyl-propanoylamino)-4,7-dihydro-5H-thieno[2,3-c]pyridine-6-carboxylic acid(pyridin-3-ylmethyl)-amide

MS: calc.: C, 24; H, 23; N, 5; O, 2; S, (445.55)
54. 3-Cyano-2-(3-cyclohexyl-propanoylamino)-4,7-dihydro-5H-thieno[2,3-c]pyridine-6-carboxylic acid(pyridin-3-ylmethyl)-amide

MS: calc.: C, 24; H, 29; N, 5; O, 2; S, (451.60). fnd.: 452.1; [M+H].
55. 3-Cyano-2-[3-(3-methoxy-phenyl)-propanoylamino]-4,7-dihydro-5H-thieno[2,3-c]pyridine-6-carboxylic acid phenethyl-amide

MS: calc.: C, 27; H, 28; N, 4; O, 3; S, (488.61). fnd.: 489.1; [M+H].
56. 3-Cyano-2-[3-(2-ethoxy-phenyl)-propanoylamino]-4,7-dihydro-5H-thieno[2,3-c]pyridine-6-carboxylic acid phenethyl-amide

MS: calc.: C, 28; H, 30; N, 4; O, 3; S, (502.64). fnd.: 503.2; [M+H].
57. 3-Cyano-2-[3-(2-methoxy-5-methyl-phenyl)-propanoylamino]-4,7-dihydro-5H-thieno[2,3-c]pyridine-6-carboxylic acid(2-pyridin-2-yl-ethyl)-amide

MS: calc.: C, 27; H, 29; N, 5; O, 3; S, (503.63). fnd.: 504.1; [M+H].
58. 3-Cyano-2-[3-(2-methoxy-phenyl)-propanoylamino]-4,7-dihydro-5H-thieno[2,3-c]pyridine-6-carboxylic acid(2-morpholin-4-yl-ethyl)-amide

MS: calc.: C, 25; H, 31; N, 5; O, 4; S, (497.62). fnd.: 498.2; [M+H].
59. 3-Cyano-2-[3-(3-methoxy-phenyl)-propanoylamino]-4,7-dihydro-5H-thieno[2,3-c]pyridine-6-carboxylic acid(2-morpholin-4-yl-ethyl)-amide

MS: calc.: C, 25; H, 31; N, 5; O, 4; S, (497.62). fnd.: 498.2; [M+H].
60. 3-Cyano-2-[3-(2-methoxy-phenyl)-propanoylamino]-4,7-dihydro-5H-thieno[2,3-c]pyridine-6-carboxylic acid(2-methoxy-ethyl)-amide

MS: calc.: C, 22; H, 26; N, 4; O, 4; S, (442.54). fnd.: 443.0; [M+H].
61. 3-Cyano-2-[3-(3-methoxy-phenyl)-propanoylamino]-4,7-dihydro-5H-thieno[2,3-c]pyridine-6-carboxylic acid(2-methoxy-ethyl)-amide

MS: calc.: C, 22; H, 26; N, 4; O, 4; S, (442.54). fnd.: 443.0; [M+H].
62. 3-Cyano-2-[3-(2-methoxy-phenyl)-propanoylamino]-4,7-dihydro-5H-thieno[2,3-c]pyridine-6-carboxylic acid(2-imidazol-1-yl-ethyl)-amide

MS: calc.: C, 24; H, 26; N, 6; O, 3; S, (478.58). fnd.: 479.1; [M+H].
63. 3-Cyano-2-[3-(3-methoxy-phenyl)-propanoylamino]-4,7-dihydro-5H-thieno[2,3-c]pyridine-6-carboxylic acid(2-imidazol-1-yl-ethyl)-amide

MS: calc.: C, 24; H, 26; N, 6; O, 3; S, (478.58). fnd.: 479.1; [M+H].
64. 3-Cyano-2-[3-(2-methoxy-phenyl)-propanoylamino]-4,7-dihydro-5H-thieno[2,3-c]pyridine-6-carboxylic acid(3-methyl-3H-imidazol-4-ylmethyl)-amide

MS: calc.: C, 24; H, 26; N, 6; O, 3; S, (478.58). fnd.: 479.2; [M+H].
65. 3-Cyano-2-[3-(3-methoxy-phenyl)-propanoylamino]-4,7-dihydro-5H-thieno[2,3-c]pyridine-6-carboxylic acid(3-methyl-3H-imidazol-4-ylmethyl)-amide

MS: calc.: C, 24; H, 26; N, 6; O, 3; S, (478.58). fnd.: 479.2; [M+H].
66. 3-Cyano-2-[3-(2-methoxy-phenyl)-propanoylamino]-4,7-dihydro-5H-thieno[2,3-c]pyridine-6-carboxylic acid(1-methyl-1H-imidazol-4-ylmethyl)-amide

MS: calc.: C, 24; H, 26; N, 6; O, 3; S, (478.58). fnd.: 479.0; [M+H].
67. 3-Cyano-2-[3-(3-methoxy-phenyl)-propanoylamino]-4,7-dihydro-5H-thieno[2,3-c]pyridine-6-carboxylic acid(1-methyl-1H-imidazol-4-ylmethyl)-amide

MS: calc.: C, 24; H, 26; N, 6; O, 3; S, (478.58). fnd.: 479.0; [M+H].
68. 3-Cyano-2-[3-(2-methoxy-phenyl)-propanoylamino]-4,7-dihydro-5H-thieno[2,3-c]pyridine-6-carboxylic acid [2-(3-methyl-3H-imidazol-4-yl)-ethyl]-amide

MS: calc.: C, 25; H, 28; N, 6; O, 3; S, (492.60)
69. 3-Cyano-2-[3-(3-methoxy-phenyl)-propanoylamino]-4,7-dihydro-5H-thieno[2,3-c]pyridine-6-carboxylic acid [2-(3-methyl-3H-imidazol-4-yl)-ethyl]-amide

MS: calc.: C, 25; H, 28; N, 6; O, 3; S, (492.60)
70. 3-Cyano-2-[3-(2-methoxy-phenyl)-propanoylamino]-4,7-dihydro-5H-thieno[2,3-c]pyridine-6-carboxylic acid [2-(1-methyl-1H-imidazol-4-yl)-ethyl]-amide

MS: calc.: C, 25; H, 28; N, 6; O, 3; S, (492.60). fnd.: 493.0; [M+H].
71. 3-Cyano-2-[3-(3-methoxy-phenyl)-propanoylamino]-4,7-dihydro-5H-thieno[2,3-c]pyridine-6-carboxylic acid [2-(1-methyl-1H-imidazol-4-yl)-ethyl]-amide

MS: calc.: C, 25; H, 28; N, 6; O, 3; S, (492.60). fnd.: 493.0; [M+H].
72. 3-Cyano-2-[3-(2-methoxy-phenyl)-propanoylamino]-4,7-dihydro-5H-thieno[2,3-c]pyridine-6-carboxylic acid(5-methyl-[1,3,4]oxadiazol-2-ylmethyl)-amide

MS: calc.: C, 23; H, 24; N, 6; O, 4; S, (480.55). fnd.: 481.0; [M+H].
73. 3-Cyano-2-[3-(3-methoxy-phenyl)-propanoylamino]-4,7-dihydro-5H-thieno[2,3-c]pyridine-6-carboxylic acid(5-methyl-[1,3,4]oxadiazol-2-ylmethyl)-amide

MS: calc.: C, 23; H, 24; N, 6; O, 4; S, (480.55). fnd.: 481.1; [M+H].
74. 3-Cyano-2-[3-(2-methoxy-phenyl)-propanoylamino]-4,7-dihydro-5H-thieno[2,3-c]pyridine-6-carboxylic acid(3-morpholin-4-yl-propyl)-amide

MS: calc.: C, 26; H, 33; N, 5; O, 4; S, (511.65). fnd.: 512.1; [M+H].
75. 3-Cyano-2-[3-(3-methoxy-phenyl)-propanoylamino]-4,7-dihydro-5H-thieno[2,3-c]pyridine-6-carboxylic acid(3-morpholin-4-yl-propyl)-amide

MS: calc.: C, 26; H, 33; N, 5; O, 4; S, (511.65). fnd.: 512.1; [M+H].
76. 3-Cyano-2-[3-(2-methoxy-phenyl)-propanoylamino]-4,7-dihydro-5H-thieno[2,3-c]pyridine-6-carboxylic acid(1-methyl-1H-pyrrol-2-ylmethyl)-amide

MS: calc.: C, 25; H, 27; N, 5; O, 3; S, (477.59). fnd.: 478.0; [M+H].
77. 3-Cyano-2-[3-(3-methoxy-phenyl)-propanoylamino]-4,7-dihydro-5H-thieno[2,3-c]pyridine-6-carboxylic acid(1-methyl-1H-pyrrol-2-ylmethyl)-amide

MS: calc.: C, 25; H, 27; N, 5; O, 3; S, (477.59). fnd.: 478.0; [M+H].
78. 3-Cyano-2-[3-(2-methoxy-phenyl)-propanoylamino]-4,7-dihydro-5H-thieno[2,3-c]pyridine-6-carboxylic acid(5-methyl-isoxazol-3-ylmethyl)-amide

MS: calc.: C, 24; H, 25; N, 5; O, 4; S, (479.56). fnd.: 480.0; [M+H].
79. 3-Cyano-2-[3-(3-methoxy-phenyl)-propanoylamino]-4,7-dihydro-5H-thieno[2,3-c]pyridine-6-carboxylic acid(5-methyl-isoxazol-3-ylmethyl)-amide

MS: calc.: C, 24; H, 25; N, 5; O, 4; S, (479.56). fnd.: 480.0; [M+H].
80. 3-Cyano-2-[3-(2-methoxy-phenyl)-propanoylamino]-4,7-dihydro-5H-thieno[2,3-c]pyridine-6-carboxylic acid(tetrahydro-furan-2-ylmethyl)-amide

MS: calc.: C, 24; H, 28; N, 4; O, 4; S, (468.58)
81. 3-Cyano-2-[3-(3-methoxy-phenyl)-propanoylamino]-4,7-dihydro-5H-thieno[2,3-c]pyridine-6-carboxylic acid(tetrahydro-furan-2-ylmethyl)-amide

MS: calc.: C, 24; H, 28; N, 4; O, 4; S, (468.58)
82. 3-Cyano-2-[3-(2-methoxy-phenyl)-propanoylamino]-4,7-dihydro-5H-thieno[2,3-c]pyridine-6-carboxylic acid carbamoylmethyl-amide

MS: calc.: C, 21; H, 23; N, 5; O, 4; S, (441.51)
83. 3-Cyano-2-[3-(3-methoxy-phenyl)-propanoylamino]-4,7-dihydro-5H-thieno[2,3-c]pyridine-6-carboxylic acid carbamoylmethyl-amide

MS: calc.: C, 21; H, 23; N, 5; O, 4; S, (441.51)
84. 3-Cyano-2-[3-(2-methoxy-phenyl)-propanoylamino]-4,7-dihydro-5H-thieno[2,3-c]pyridine-6-carboxylic acid(2,5-dimethyl-2H-pyrazol-3-ylmethyl)-amide

MS: calc.: C, 25; H, 28; N, 6; O, 3; S, (492.60). fnd.: 493.2; [M+H].
85. 3-Cyano-2-[3-(3-methoxy-phenyl)-propanoylamino]-4,7-dihydro-5H-thieno[2,3-c]pyridine-6-carboxylic acid(2,5-dimethyl-2H-pyrazol-3-ylmethyl)-amide

MS: calc.: C, 25; H, 28; N, 6; O, 3; S, (492.60). fnd.: 493.2; [M+H].
86. 3-Cyano-2-[3-(2-methoxy-phenyl)-propanoylamino]-4,7-dihydro-5H-thieno[2,3-c]pyridine-6-carboxylic acid(1-methyl-1H-pyrrol-2-ylmethyl)-amide

MS: calc.: C, 25; H, 27; N, 5; O, 3; S, (477.59)
87. 3-Cyano-2-[3-(3-methoxy-phenyl)-propanoylamino]-4,7-dihydro-5H-thieno[2,3-c]pyridine-6-carboxylic acid(1-methyl-1H-pyrrol-2-ylmethyl)-amide

MS: calc.: C, 25; H, 27; N, 5; O, 3; S, (477.59)
88. 3-Cyano-2-[3-(2-methoxy-phenyl)-propanoylamino]-4,7-dihydro-5H-thieno[2,3-c]pyridine-6-carboxylic acid(1,3-dimethyl-1H-pyrazol-4-ylmethyl)-amide

MS: calc.: C, 25; H, 28; N, 6; O, 3; S, (492.60). fnd.: 493.2; [M+H].
89. 3-Cyano-2-[3-(3-methoxy-phenyl)-propanoylamino]-4,7-dihydro-5H-thieno[2,3-c]pyridine-6-carboxylic acid(1,3-dimethyl-1H-pyrazol-4-ylmethyl)-amide

MS: calc.: C, 25; H, 28; N, 6; O, 3; S, (492.60). fnd.: 493.2; [M+H].
90. 3-Cyano-2-[3-(2-methoxy-phenyl)-propanoylamino]-4,7-dihydro-5H-thieno[2,3-c]pyridine-6-carboxylic acid(isoxazol-3-ylmethyl)-amide

MS: calc.: C, 23; H, 23; N, 5; O, 4; S, (465.53)
91. 3-Cyano-2-[3-(3-methoxy-phenyl)-propanoylamino]-4,7-dihydro-5H-thieno[2,3-c]pyridine-6-carboxylic acid(isoxazol-3-ylmethyl)-amide

MS: calc.: C, 23; H, 23; N, 5; O, 4; S, (465.53)
92. 3-Cyano-2-[3-(2-methoxy-phenyl)-propanoylamino]-4,7-dihydro-5H-thieno[2,3-c]pyridine-6-carboxylic acid(3-methyl-isoxazol-5-ylmethyl)-amide

MS: calc.: C, 24; H, 25; N, 5; O, 4; S, (479.56)
93. 3-Cyano-2-[3-(3-methoxy-phenyl)-propanoylamino]-4,7-dihydro-5H-thieno[2,3-c]pyridine-6-carboxylic acid(3-methyl-isoxazol-5-ylmethyl)-amide

MS: calc.: C, 24; H, 25; N, 5; O, 4; S, (479.56)
94. 3-Cyano-2-[3-(2-methoxy-phenyl)-propanoylamino]-4,7-dihydro-5H-thieno[2,3-c]pyridine-6-carboxylic acid(oxazol-2-ylmethyl)-amide

MS: calc.: C, 23; H, 23; N, 5; O, 4; S, (465.53)
95. 3-Cyano-2-[3-(3-methoxy-phenyl)-propanoylamino]-4,7-dihydro-5H-thieno[2,3-c]pyridine-6-carboxylic acid(oxazol-2-ylmethyl)-amide

MS: calc.: C, 23; H, 23; N, 5; O, 4; S, (465.53)
96. 3-Cyano-2-[3-(2-methoxy-phenyl)-propanoylamino]-4,7-dihydro-5H-thieno[2,3-c]pyridine-6-carboxylic acid(1-methyl-1H-pyrazol-4-ylmethyl)-amide

MS: calc.: C, 24; H, 26; N, 6; O, 3; S, (478.58). fnd.: 479.2; [M+H].
97. 3-Cyano-2-[3-(3-methoxy-phenyl)-propanoylamino]-4,7-dihydro-5H-thieno[2,3-c]pyridine-6-carboxylic acid(1-methyl-1H-pyrazol-4-ylmethyl)-amide

MS: calc.: C, 24; H, 26; N, 6; O, 3; S, (478.58). fnd.: 479.2; [M+H].
98. 3-Cyano-2-[3-(3-methoxy-phenyl)-propanoylamino]-4,7-dihydro-5H-thieno[2,3-c]pyridine-6-carboxylic acid(2-methyl-2H-pyrazol-3-ylmethyl)-amide

MS: calc.: C, 24; H, 26; N, 6; O, 3; S, (478.58). fnd.: 479.2; [M+H].
99. 3-Cyano-2-[3-(2-methoxy-phenyl)-propanoylamino]-4,7-dihydro-5H-thieno[2,3-c]pyridine-6-carboxylic acid(2-methyl-2H-pyrazol-3-ylmethyl)-amide

MS: calc.: C, 24; H, 26; N, 6; O, 3; S, (478.58). fnd.: 479.2; [M+H].
100. 3-Cyano-2-[3-(2-methoxy-phenyl)-propanoylamino]-4,7-dihydro-5H-thieno[2,3-c]pyridine-6-carboxylic acid(1-methyl-1H-imidazol-2-ylmethyl)-amide

MS: calc.: C, 24; H, 26; N, 6; O, 3; S, (478.58). fnd.: 479.2; [M+H].
101. 3-Cyano-2-[3-(3-methoxy-phenyl)-propanoylamino]-4,7-dihydro-5H-thieno[2,3-c]pyridine-6-carboxylic acid(1-methyl-1H-imidazol-2-ylmethyl)-amide

MS: calc.: C, 24; H, 26; N, 6; O, 3; S, (478.58). fnd.: 479.2; [M+H].
102. 3-Cyano-2-[3-(3-methoxy-phenyl)-propanoylamino]-4,7-dihydro-5H-thieno[2,3-c]pyridine-6-carboxylic acid(3-methyl-1H-pyrazol-4-ylmethyl)-amide

MS: calc.: C, 24; H, 26; N, 6; O, 3; S, (478.58)
103. 3-Cyano-2-[3-(2-methoxy-phenyl)-propanoylamino]-4,7-dihydro-5H-thieno[2,3-c]pyridine-6-carboxylic acid(3-methyl-1H-pyrazol-4-ylmethyl)-amide

MS: calc.: C, 24; H, 26; N, 6; O, 3; S, (478.58)
104. 3-Cyano-2-[3-(3-methoxy-phenyl)-propanoylamino]-4,7-dihydro-5H-thieno[2,3-c]pyridine-6-carboxylic acid [2-(1-methyl-1H-imidazol-4-yl)-ethyl]-amide

MS: calc.: C, 25; H, 28; N, 6; O, 3; S, (492.60)
105. 3-Cyano-2-[3-(2-methoxy-phenyl)-propanoylamino]-4,7-dihydro-5H-thieno[2,3-c]pyridine-6-carboxylic acid [2-(1-methyl-1H-imidazol-4-yl)-ethyl]-amide

MS: calc.: C, 25; H, 28; N, 6; O, 3; S, (492.60)

In a different approach (General Procedure B) the following ethyl ureas can be prepared as described as follows:

2.5 eq. of ethyl isocyanate is dissolved in dichloromethane and 1 eq. of the appropriate amino building block A1-A24 is added and the reaction mixture stirred over night. In case the reaction is not complete, 1.5 eq of triethylamine is added and the reaction is evaporated after another several hours. The reaction mixture is evaporated and the desired product is obtained after crystallization from ethanol. In case crystallization does not give sufficiently pure product, the evaporateed reaction mixture or impure product is subjected to flash chromatography or HPLC with or without subsequent cystallization.

Using this procedure the following compounds may be prepared:
106. 3-Cyano-2-[3-(2-ethoxy-phenyl)-propanoylamino]-4,7-dihydro-5H-thieno[2,3-c]pyridine-6-carboxylic acid ethylamide

MS: calc.: C, 22; H, 26; N, 4; O, 3; S, (426.54). fnd.: 427.1; [M+H].
107. 3-Cyano-2-[3-(2-methoxy-5-methyl-phenyl)-propanoylamino]-4,7-dihydro-5H-thieno[2,3-c]pyridine-6-carboxylic acid ethylamide

MS: calc.: C, 22; H, 26; N, 4; O, 3; S, (426.54). fnd.: 427.2; [M+H].
108. 3-Cyano-2-[3-(3-methoxy-phenyl)-propanoylamino]-4,7-dihydro-5H-thieno[2,3-c]pyridine-6-carboxylic acid ethylamide

MS: calc.: C, 21; H, 24; N, 4; O, 3; S, (412.51). fnd.: 413.0; [M+H].
109. 3-Cyano-2-(3-furan-2-yl-propanoylamino)-4,7-dihydro-5H-thieno[2,3-c]pyridine-6-carboxylic acid ethylamide

MS: calc.: C, 18; H, 20; N, 4; O, 3; S, (372.45). fnd.: 373.0; [M+H].
110. 3-Cyano-2-[3-(3-methoxy-phenyl)-butanoylamino]-4,7-dihydro-5H-thieno[2,3-c]pyridine-6-carboxylic acid ethylamide

MS: calc.: C, 22; H, 26; N, 4; O, 3; S, (426.54). fnd.: 427.2; [M+H].
111. 3-Cyano-2-[3-(2-methoxy-phenyl)-butanoylamino]-4,7-dihydro-5H-thieno[2,3-c]pyridine-6-carboxylic acid ethylamide

MS: calc.: C, 22; H, 26; N, 4; O, 3; S, (426.54). fnd.: 427.2; [M+H].
112. 3-Cyano-2-[3-(2-ethoxy-phenyl)-butanoylamino]-4,7-dihydro-5H-thieno[2,3-c]pyridine-6-carboxylic acid ethylamide

MS: calc.: C, 23; H, 28; N, 4; O, 3; S, (440.57). fnd.: 441.2; [M+H].
113. 3-Cyano-2-[3-(2-methoxy-phenyl)-propanoylamino]-4,7-dihydro-5H-thieno[2,3-c]pyridine-6-carboxylic acid ethylamide

MS: calc.: C, 21; H, 24; N, 4; O, 3; S, (412.51). fnd.: 413.2; [M+H].
114. 3-Cyano-2-(3-phenyl-butanoylamino)-4,7-dihydro-5H-thieno[2,3-c]pyridine-6-carboxylic acid ethylamide

MS: calc.: C, 21; H, 24; N, 4; O, 2; S, (396.52). fnd.: 397.2; [M+H].
115. 3-Cyano-2-(3-phenyl-propanoylamino)-4,7-dihydro-5H-thieno[2,3-c]pyridine-6-carboxylic acid ethylamide

MS: calc.: C, 20; H, 22; N, 4; O, 2; S, (382.49). fnd.: 483.1; [M+H].
116. 3-Cyano-2-({1-[(RS)-2-(2-methoxy-phenyl)-cyclopropyl]-methanoyl}-amino)-4,7-dihydro-5H-thieno[2,3-c]pyridine-6-carboxylic acid ethylamide

MS: calc.: C, 22; H, 24; N, 4; O, 3; S, (424.53). fnd.: 424.9; [M+H].
117. 3-Cyano-2-({1-[(RS)-2-(3-methoxy-phenyl)-cyclopropyl]-methanoyl}-amino)-4,7-dihydro-5H-thieno[2,3-c]pyridine-6-carboxylic acid ethylamide

MS: calc.: C, 22; H, 24; N, 4; O, 3; S, (424.53). fnd.: 425.0; [M+H].
118. 3-Cyano-2-(3-cyclohexyl-propanoylamino)-4,7-dihydro-5H-thieno[2,3-c]pyridine-6-carboxylic acid ethylamide

MS: calc.: C, 20; H, 28; N, 4; O, 2; S, (388.54). fnd.: 489.0; [M+H].

### Starting Materials:

A1. N-(3-Cyano-4,5,6,7-tetrahydro-thieno[2,3-c]pyridin-2-yl)-3-(3-methoxy-phenyl)-propionamide

2.59 g of Boc-4-piperidinone and 0.86 g of malonitrile are dissolved in 5.5 ml of ethanol and 0.42 g finely ground sulfur is added. After addition of 1.04 ml of diethyl amine the reaction mixture gets hot within seconds and is refluxed for several minutes until complete dissolution took place. Upon cooling to room temperature the reaction mixture solidifies over night. Addition of ˜20 ml ethanol affords a suspension, which can be poured on ice water to yield fine powder (after stirring for about one hour) which can be separated by filtration. Recrystallization from ethanol affords 3.12 g (86%) of sufficiently pure 2-Amino-3-cyano-4,7-dihydro-5H-thieno[2,3-c]pyridine-6-carboxylic acid tert-butyl ester.

0.14 g of 3-(3-methoxy-phenyl) propionic acid are dissolved in 0.8 ml dichloromethane and 0.12 g of CDI are added. After the gas evolution has deceased 0.25 ml triethylamine and 0.1 g 2-Amino-3-cyano-4,7-dihydro-5H-thieno[2,3-c]pyridine-6-carboxylic acid tert-butyl ester are added and the reaction mixture stirred at room temperature for several days. The reaction mixture is evaporated and the remaining residue crystallizes from ethanol to yield 0.15 g of 3-Cyano-2-[3-(2-methoxy-phenyl)-propanoylamino]-4,7-dihydro-5H-thieno[2,3-c]pyridine-6-carboxylic acid tert-butyl ester (mp 198° C., Rf=0.75 DCM/MeOH=95/5). In case the crystallization do not afford sufficiently pure product, a flash chromatography using a dichloromethane/methanol gradient is used for purification.

0.15 g of 3-Cyano-2-[3-(2-methoxy-phenyl)-propanoylamino]-4,7-dihydro-5H-thieno[2,3-c]pyridine-6-carboxylic acid tert-butyl ester are suspended in 1 ml dichloromethane, 0.4 ml trifluoro acetic acid are added and the reaction mixture stirred for several hours. Removal of the solvents and recrystallization from ethanol affords 0.15 of N-(3-Cyano-4,5,6,7-tetrahydro-thieno[2,3-c]pyridin-2-yl)-3-(2-methoxy-phenyl)-propionamide as its trifluoro acetate salt (mp dec., Rf=0.4 DCM/MeOH=90/10).

The following starting materials may be prepared according to this procedure. In case a product is not sufficiently pure a flash chromatography or HPLC may be performed.
A2. N-(3-Cyano-4,5,6,7-tetrahydro-thieno[2,3-c]pyridin-2-yl)-3-(2-methoxy-phenyl)-propionamide
A3. (RS)—N-(3-Cyano-4,5,6,7-tetrahydro-thieno[2,3-c]pyridin-2-yl)-3-(2-methoxy-phenyl)-butyramide
A4. (RS)—N-(3-Cyano-4,5,6,7-tetrahydro-thieno[2,3-c]pyridin-2-yl)-3-(2-ethoxy-phenyl)-butyramide
A5. (1RS,2RS)-2-(3-Methoxy-phenyl)-cyclopropanecarboxylic acid(3-cyano-4,5,6,7-tetrahydro-thieno[2,3-c]pyridin-2-yl)-amide
A6. (1RS,2RS)-2-(2-Methoxy-phenyl)-cyclopropanecarboxylic acid(3-cyano-4,5,6,7-tetrahydro-thieno[2,3-c]pyridin-2-yl)-amide
A7. N-(3-Cyano-4,5,6,7-tetrahydro-thieno[2,3-c]pyridin-2-yl)-3-phenyl-propionamide
A8. (RS)—N-(3-Cyano-4,5,6,7-tetrahydro-thieno[2,3-c]pyridin-2-yl)-3-phenyl-butyramide
A9. (RS)—N-(3-Cyano-4,5,6,7-tetrahydro-thieno[2,3-c]pyridin-2-yl)-3-cyclohexyl-butyramide
A10. N-(3-Cyano-4,5,6,7-tetrahydro-thieno[2,3-c]pyridin-2-yl)-3-(2-methoxy-5-methyl-phenyl)-propionamide
A11. N-(3-Cyano-4,5,6,7-tetrahydro-thieno[2,3-c]pyridin-2-yl)-3-furan-2-yl-propionamide
A12. N-(3-Cyano-4,5,6,7-tetrahydro-thieno[2,3-c]pyridin-2-yl)-3-(2-ethoxy-phenyl)-propionamide
A13. N-(3-Cyano-4,5,6,7-tetrahydro-thieno[2,3-c]pyridin-2-yl)-3-pyridin-2-yl-propionamide
A14. N-(3-Cyano-4,5,6,7-tetrahydro-thieno[2,3-c]pyridin-2-yl)-3-pyridin-3-yl-propionamide
A15. N-(3-Cyano-4,5,6,7-tetrahydro-thieno[2,3-c]pyridin-2-yl)-3-cyclohexyl-propionamide
A16. (1RS,2RS)-2-(2-Ethoxy-phenyl)-cyclopropanecarboxylic acid(3-cyano-4,5,6,7-tetrahydro-thieno[2,3-c]pyridin-2-yl)-amide
A17. (1RS,2RS)-2-Pyridin-2-yl-cyclopropanecarboxylic acid(3-cyano-4,5,6,7-tetrahydro-thieno[2,3-c]pyridin-2-yl)-amide
A18. (1RS,2RS)-2-Pyridin-3-yl-cyclopropanecarboxylic acid(3-cyano-4,5,6,7-tetrahydro-thieno[2,3-c]pyridin-2-yl)-amide
A19. (1RS,2RS)-2-Furan-2-yl-cyclopropanecarboxylic acid(3-cyano-4,5,6,7-tetrahydro-thieno[2,3-c]pyridin-2-yl)-amide
A20. (1RS,2RS)-2-Phenyl-cyclopropanecarboxylic acid(3-cyano-4,5,6,7-tetrahydro-thieno[2,3-c]pyridin-2-yl)-amide
A21. (RS)—N-(3-Cyano-4,5,6,7-tetrahydro-thieno[2,3-c]pyridin-2-yl)-3-(3-methoxy-phenyl)-butyramide
A22. (RS)—N-(3-Cyano-4,5,6,7-tetrahydro-thieno[2,3-c]pyridin-2-yl)-3-pyridin-2-yl-butyramide
A23. (RS)—N-(3-Cyano-4,5,6,7-tetrahydro-thieno[2,3-c]pyridin-2-yl)-3-pyridin-3-yl-butyramide
A24. (RS)—N-(3-Cyano-4,5,6,7-tetrahydro-thieno[2,3-c]pyridin-2-yl)-3-furan-2-yl-butyramide

### Further General Procedures:

### General Procedure for the Preparation of Carboxylic Acids

Synthesis of Propionic Acids/Acrylic Acids Starting from Aldehyde:

10 mmol of the appropriate aldehyde are dissolved with 1.1 eq. of triethyl phosphonoacetate in 7 ml THF. At 0° C. 1 eq. of DBU is added and the reaction mixture is stirred over night at room temperature. Then, the reaction mixture is diluted with water, acidified with aq. HCl and extracted with diethyl ether. The organic layer is dried over MgSO₄ and the solvent removed. This acrylic acid ester is used without further purification. The crude acrylic acid ester is suspended in 20 ml 1N NaOH and stirred over night. After the reaction is completed, the reaction mixture is acidified with 1N HCl and extracted with diethyl ether. The organic layer is dried over MgSO₄ and the solvent evaporated; the desired acrylic acid is obtained in almost pure form.

11 mmol of the acrylic acid are dissolved in 20 ml MeOH, 1 eq. of NaHCO₃ and 200 mg Pd/C (10%) are added and the reaction hydrogenated over night at room temperature and normal pressure. Filtration of the reaction mixture over Celite and removal of the solvent affords the desired product in good yield in pure form. In case one of the products is not sufficiently pure, one can also purify them via flash chromatography. According to the above-mentioned procedure, the following compound can be prepared: 2 g of 2-methoxy-5-methyl-benaldehyde is transformed to 2.2 g of (2-methoxy-5-methyl-phenyl)-acrylic acid. 21 g of the before-mentioned acrylic acid are hydrogenated to yield 20 g of the desired 3-(2-methoxy-5-methyl-phenyl)-propionic acid. Further relevant starting compounds can be prepared similarly, such as e.g. 3-(2-methoxy-phenyl)-propionic acid, 3-(2-ethoxy-phenyl)-propionic acid or 3-(3-methoxy-phenyl)-propionic acid.

Synthesis of β-methyl propionic acid starting from acetophenone:

1.9 mmol of sodium hydride are suspended in 5 ml toluene and 1.6 mmol triethyl phosphonoacetate are added at 0° C. After stirring for 30 min at 0° C., 1.1 mmol of the appropriate acetophenone is dissolved in 1 ml toluene, added to the reaction mixture and the reaction mixture stirred over night or for several days at room temperature or heated to 60° C. After addition of some water, the reaction mixture is extracted with toluene and the combined organic layers are dried over MgSO₄. The crude acrylic acid ester is obtained as cis/trans mixture and used without further purification. The acrylic acid ester is suspended in a mixture of EtOH and 1N NaOH and stirred over night at room temperature. After acidification with 1N HCl the acrylic acid crystallizes and can be obtained by filtration. In case no crystallization can be achieved, the acrylic acid can be purified via flash chromatography. The acrylic acid is hydrogenated in MeOH with Pd/C (10%) and 1 eq. NaHCO₃ under normal pressure at room temperature. After filtration over Celite, the solvent is removed and the desired β-methyl propionic acid purified via flash chromatography if necessary. According to the above-mentioned procedure, the following compound can be prepared: Starting from 180 mg 2-methoxy-5-methyl-acetophenone, 75 mg of 2-methoxy-5-methyl crotonic acid can be obtained as cis/trans mixture. Hydrogenation of 200 mg of the crotonic acid affords 190 mg of the 3-(2-methoxy-5-methyl-phenyl)-butyric acid. Further relevant starting compounds can be prepared similarly, such as e.g. 3-(2-ethoxy-phenyl)-butyric acid from 2-ethoxy-acetophenone or, accordingly, 3-(2-methoxy-phenyl)-butyric acid or 3-(3-methoxy-phenyl)-butyric acid.

### Cyclopropanation:

113 mg of sodium hydride and 1.1 g of trimethyl sulfoxonium iodide are stirred for one hour in 7 ml DMSO at room temperature. 500 mg of trans cinnamic acid ethyl ester are dissolved in 6 ml DMSO/THF (1:1) and added to the reaction mixture. After completion of the reaction (3 h, TLC) 1N HCl is added and the reaction mixture extracted with diethyl ether. The combined organic layers are dried over MgSO₄, the solvent removed and the crude product (393 mg) is used without further purification. In case the purity is not sufficient, the product can be purified by flash chromatography. Saponification of the ester to give the corresponding carboxylic acid can be obtained similarly as described in the foregoing procedures. Further relevant starting compounds can be obtained similarly. Thus, e.g. 2-(pyridin-2-yl)-cyclopropanecarboxylic acid, 2-(pyridin-3-yl)-cyclopropanecarboxylic acid, 2-(furan-2-yl)-cyclopropanecarboxylic acid and 2-cyclohexyl-cyclopropanecarboxylic acid may be obtained similarly.

### 2-(3-Methoxy-phenyl)-cyclopropanecarboxylic acid

9.7 g of 3-methoxycinnamic acid are suspended in 100 ml EtOH and 4 ml H₂SO₄. After stirring over night, the solvent is evaporated and 100 ml ice water added. Neutralization and extraction with dichloromethane followed by removal of the solvent affords the ethyl ester in almost quantitative yield. This crude product is used without further purification.

2 g sodium hydride and 22 g trimethylsulfoxonium chloride are suspended in ˜50 ml DMSO and after gas evolution has deceased 11.5 g of the above 3-methoxycinnamic acid ethyl ester in 20 ml DMSO/THF are added. After stirring for several days, 1N HCl is added under ice cooling and the mixture is extracted with diethylether. The combined organic phases are dried over MgSO₄ and the solvent removed. This crude product is used without further purification.

The crude 2-(3-methoxy-phenyl)-cyclopropanecarboxylic acid ethyl ester is dissolved in 30 ml EtOH and 15 ml 1N NaOH. After stirring over night, the reaction mixture is acidified with 1N HCl and extracted with diethyl ether. After removal of the solvent 7.1 g of the 2-(3-methoxy-phenyl)-cyclopropanecarboxylic acid is obtained. This 2-(3-methoxy-phenyl)-cyclopropanecarboxylic acid is used without further purification.

2-(2-Methoxy-phenyl)-cyclopropanecarboxylic acid, 2-(2-ethoxy-phenyl)-cyclopropanecarboxylic acid and 2-(2-methoxy-5-methyl-phenyl)-cyclopropanecarboxylic acid may be obtained similarly.

### 3-Pyridin-2-yl-butyric acid

The title compound can be obtained from the corresponding methyl ester, which is described e.g. in Lindstedt E.-L., Nilsson M., Acta Chem. Scand. Ser. B, EN, 40, 6, 1986, 466-469, by standard saponification using e.g. NaOH or LiOH.

### 3-Pyridin-3-yl-butyric acid

The title compound can be obtained from the corresponding ethyl ester, which is described e.g. in Sainsbury M., Weerasinghe D., Dolman D., J. Chem. Soc. Perkin Trans. 1, EN, 1982, 587-590, by standard saponification using e.g. NaOH or LiOH.

3-Phenyl-butyric acid, 3-cyclohexyl-butyric acid and 3-(furan-2-yl)-butyric acid can be obtained from the corresponding acetophenone similarly as described above.

3-Cyclohexyl-propionic acid is known or can be obtained analogously or similarly to known procedures.

Relevant 3-pyridyl-propionic acids, 3-furyl-propionic acids, 3-pyridyl-acrylic acids, 3-furyl-acrylic acids or other relevant propionic acid/acrylic acid derivatives are known or can be obtained analogously or similarly to known procedures.

Further compounds according to the present invention that can be obtained according to the procedures mentioned above include:
119. 3-Cyano-2-[3-(3-methoxy-phenyl)-propanoylamino]-4,7-dihydro-5H-thieno[2,3-c]pyridine-6-carboxylic acid(2-pyridin-3-yl-ethyl)-amide

MS: calc.: C, 26; H, 27; N, 5; O, 3; S, (489.6). fnd.: 490.1; [M+H].
120. 3-Cyano-2-[3-(2-methoxy-phenyl)-propanoylamino]-4,7-dihydro-5H-thieno[2,3-c]pyridine-6-carboxylic acid(6-trifluoromethyl-pyridin-3-ylmethyl)-amide

MS: calc.: C₂₆H₂₄F₃N₅O₃S (543.57). fnd.: 544.0; [M+H].
121. 3-Cyano-2-{[1-(2-phenyl-cyclopropyl)-methanoyl]-amino}-4,7-dihydro-5H-thieno[2,3-c]pyridine-6-carboxylic acid(2-pyridin-4-yl-ethyl)-amide

MS: calc.: C, 26; H, 25; N, 5; O, 2; S, (471.59). fnd.: 472.0; [M+H].
122. 3-Cyano-2-{[1-(2-phenyl-cyclopropyl)-methanoyl]-amino}-4,7-dihydro-5H-thieno[2,3-c]pyridine-6-carboxylic acid(pyridin-2-ylmethyl)-amide

MS: calc.: C, 25; H, 23; N, 5; O, 2; S, (457.56). fnd.: 457.8; [M+H].
123. N-[3-Cyano-6-(1-imidazol-1-yl-methanoyl)-4,5,6,7-tetrahydro-thieno[2,3-c]pyridin-2-yl]-3-(2-ethoxy-phenyl)-propionamide

MS: calc.: C, 23; H, 23; N, 5; O, 3; S, (449.54). fnd.: 450.1; [M+H].
124. N-[3-Cyano-6-(1-imidazol-1-yl-methanoyl)-4,5,6,7-tetrahydro-thieno[2,3-c]pyridin-2-yl]-3-furan-2-yl-propionamide

MS: calc.: C, 19; H, 17; N, 5; O, 3; S, (395.44). fnd.: 396.1; [M+H].
125. N-[3-Cyano-6-(1-imidazol-1-yl-methanoyl)-4,5,6,7-tetrahydro-thieno[2,3-c]pyridin-2-yl]-3-phenyl-propionamide

MS: calc.: C, 21; H, 19; N, 5; O, 2; S, (405.48). fnd.: 405.9; M+H].
126. 3-Cyano-2-[3-(2-methoxy-phenyl)-propanoylamino]-4,7-dihydro-5H-thieno[2,3-c]pyridine-6-carboxylic acid(5-tert-butyl-2-methyl-2H-pyrazol-3-ylmethyl)-amide

MS: calc.: C, 28; H, 34; N, 6; O, 3; S, (534.69). fnd.: 535.1; [M+H].
127. 3-Cyano-2-[3-(3-methoxy-phenyl)-propanoylamino]-4,7-dihydro-5H-thieno[2,3-c]pyridine-6-carboxylic acid(pyrazin-2-ylmethyl)-amide

MS: calc.: C, 24; H, 24; N, 6; O, 3; S, (476.56). fnd.: 476.9; [M+H].
128. 3-Cyano-2-[3-(3-methoxy-phenyl)-propanoylamino]-4,7-dihydro-5H-thieno[2,3-c]pyridine-6-carboxylic acid(6-methoxy-pyridin-3-ylmethyl)-amide

MS: calc.: C, 26; H, 27; N, 5; O, 4; S, (505.6). fnd.: 506.1; [M+H].
129. 3-Cyano-2-[3-(3-methoxy-phenyl)-propanoylamino]-4,7-dihydro-5H-thieno[2,3-c]pyridine-6-carboxylic acid(2,4-dimethyl-thiazol-5-ylmethyl)-amide

MS: calc.: C, 25; H, 27; N, 5; O, 3; S, 2; (509.65). fnd.: 510.1; [M+H].
130. 3-Cyano-2-[3-(3-methoxy-phenyl)-propanoylamino]-4,7-dihydro-5H-thieno[2,3-c]pyridine-6-carboxylic acid(2-methyl-thiazol-4-ylmethyl)-amide

MS: calc.: C, 24; H, 25; N, 5; O, 3; S, 2; (495.63). fnd.: 496.0; [M+H].
131. 3-Cyano-2-{[1-(2-phenyl-cyclopropyl)-methanoyl]-amino}-4,7-dihydro-5H-thieno[2,3-c]pyridine-6-carboxylic acid(pyridin-3-ylmethyl)-amide

MS: calc.: C, 25; H, 23; N, 5; O, 2; S, (457.56). fnd.: 458.1; [M+H].
132. 3-Cyano-2-{[1-(2-phenyl-cyclopropyl)-methanoyl]-amino}-4,7-dihydro-5H-thieno[2,3-c]pyridine-6-carboxylic acid(pyridin-4-ylmethyl)-amide

MS: calc.: C, 25; H, 23; N, 5; O, 2; S, (457.56). fnd.: 458.1; [M+H].
133. 3-Cyano-2-{[1-(2-phenyl-cyclopropyl)-methanoyl]-amino}-4,7-dihydro-5H-thieno[2,3-c]pyridine-6-carboxylic acid(2-pyridin-3-yl-ethyl)-amide

MS: calc.: C, 26; H, 25; N, 5; O, 2; S, (471.59). fnd.: 472.1; [M+H].
134. 3-Cyano-2-[3-(2-ethoxy-phenyl)-butanoylamino]-4,7-dihydro-5H-thieno[2,3-c]pyridine-6-carboxylic acid(2-pyridin-3-yl-ethyl)-amide

MS: calc.: C, 28; H, 31; N, 5; O, 3; S, (517.65). fnd.: 518.2; [M+H].
135. 3-Cyano-2-[3-(3-methoxy-phenyl)-propanoylamino]-4,7-dihydro-5H-thieno[2,3-c]pyridine-6-carboxylic acid(2-methoxy-pyridin-4-ylmethyl)-amide

MS: calc.: C, 26; H, 27; N, 5; O, 4; S, (505.6). fnd.: 506.1; [M+H].
136. 3-Cyano-2-[3-(3-methoxy-phenyl)-propanoylamino]-4,7-dihydro-5H-thieno[2,3-c]pyridine-6-carboxylic acid(isoxazol-5-ylmethyl)-amide

MS: calc.: C, 23; H, 23; N, 5; O, 4; S, (465.53). fnd.: 466.0; [M+H].
137. 3-Cyano-2-[3-(2-methoxy-phenyl)-propanoylamino]-4,7-dihydro-5H-thieno[2,3-c]pyridine-6-carboxylic acid(6-methoxy-pyridin-3-ylmethyl)-amide

MS: calc.: C, 26; H, 27; N, 5; O, 4; S, (505.6). fnd.: 506.1; [M+H].
138. 3-Cyano-2-[3-(2-ethoxy-phenyl)-butanoylamino]-4,7-dihydro-5H-thieno[2,3-c]pyridine-6-carboxylic acid(pyridin-3-ylmethyl)-amide

MS: calc.: C, 27; H, 29; N, 5; O, 3; S, (503.63). fnd.: 504.3; [M+H].
139. 3-Cyano-2-[3-(2-methoxy-phenyl)-propanoylamino]-4,7-dihydro-5H-thieno[2,3-c]pyridine-6-carboxylic acid(2-methyl-thiazol-4-ylmethyl)-amide

MS: calc.: C, 24; H, 25; N, 5; O, 3; S, 2; (495.63). fnd.: 495.9; [M+H].
140. 3-Cyano-2-[3-(2-methoxy-phenyl)-propanoylamino]-4,7-dihydro-5H-thieno[2,3-c]pyridine-6-carboxylic acid(pyrazin-2-ylmethyl)-amide

MS: calc.: C, 24; H, 24; N, 6; O, 3; S, (476.56). fnd.: 477.0; [M+H].
141. 3-Cyano-2-[3-(2-methoxy-phenyl)-propanoylamino]-4,7-dihydro-5H-thieno[2,3-c]pyridine-6-carboxylic acid(2,4-dimethyl-thiazol-5-ylmethyl)-amide

MS: calc.: C, 25; H, 27; N, 5; O, 3; S, 2; (509.65). fnd.: 510.0; [M+H].
142. 3-Cyano-2-[3-(3-methoxy-phenyl)-propanoylamino]-4,7-dihydro-5H-thieno[2,3-c]pyridine-6-carboxylic acid(thiazol-2-ylmethyl)-amide

MS: calc.: C, 23; H, 23; N, 5; O, 3; S, 2; (481.6). fnd.: 481.9; [M+H].
143. 3-Cyano-2-[3-(3-methoxy-phenyl)-propanoylamino]-4,7-dihydro-5H-thieno[2,3-c]pyridine-6-carboxylic acid(5-methyl-isoxazol-4-ylmethyl)-amide

MS: calc.: C, 24; H, 25; N, 5; O, 4; S, (479.56). fnd.: 480.0; [M+H].
144. 3-Cyano-2-[3-(2-methoxy-phenyl)-propanoylamino]-4,7-dihydro-5H-thieno[2,3-c]pyridine-6-carboxylic acid(isoxazol-5-ylmethyl)-amide

MS: calc.: C, 23; H, 23; N, 5; O, 4; S, (465.53). fnd.: 466.0; [M+H].
145. 3-Cyano-2-[3-(2-methoxy-phenyl)-propanoylamino]-4,7-dihydro-5H-thieno[2,3-c]pyridine-6-carboxylic acid(2-methoxy-pyridin-4-ylmethyl)-amide

MS: calc.: C, 26; H, 27; N, 5; O, 4; S, (505.6). fnd.: 506.0; [M+H].
146. 3-Cyano-2-[3-(3-methoxy-phenyl)-propanoylamino]-4,7-dihydro-5H-thieno[2,3-c]pyridine-6-carboxylic acid(3-methyl-isoxazol-4-ylmethyl)-amide

MS: calc.: C, 24; H, 25; N, 5; O, 4; S, (479.56). fnd.: 480.0; [M+H].
147. 3-Cyano-2-[3-(2-methoxy-phenyl)-propanoylamino]-4,7-dihydro-5H-thieno[2,3-c]pyridine-6-carboxylic acid(3-methyl-isoxazol-4-ylmethyl)-amide

MS: calc.: C, 24; H, 25; N, 5; O, 4; S, (479.56). fnd.: 480.0; [M+H].
148. 3-Cyano-2-[3-(2-methoxy-phenyl)-propanoylamino]-4,7-dihydro-5H-thieno[2,3-c]pyridine-6-carboxylic acid(5-methyl-isoxazol-4-ylmethyl)-amide

MS: calc.: C, 24; H, 25; N, 5; O, 4; S, (479.56). fnd.: 480.0; [M+H].
149. 3-Cyano-2-[3-(2-methoxy-phenyl)-propanoylamino]-4,7-dihydro-5H-thieno[2,3-c]pyridine-6-carboxylic acid(thiazol-2-ylmethyl)-amide

MS: calc.: C, 23; H, 23; N, 5; O, 3; S, 2; (481.6). fnd.: 481.9; [M+H].

### Commercial Utility

The compounds according to the present invention have miscellaneous valuable pharmacological properties which make them commercially useful.

The compounds according to the invention therefore can be employed as therapeutic agents for the treatment and prophylaxis of diseases in human and veterinary medicine.

Thus, for example, in more embodimental detail, the compounds according to this invention are potent and highly efficacious cell-cycle specific inhibitors of cellular (hyper)proliferation and/or inducers of apoptosis in cancer cells. Therefore, these compounds are expected to be useful for treating (hyper)proliferative diseases and/or disorders responsive to the induction of apoptosis, in particular cancer.

Further on, these compounds can be useful in the treatment of benign or malignant neoplasia.

A “neoplasia” is defined by cells displaying aberrant cell proliferation and/or survival and/or a block in differentiation. A “benign neoplasia” is described by hyperproliferation of cells, incapable of forming an aggressive, metastasizing tumor in-vivo. In contrast, a “malignant neoplasia” is described by cells with multiple cellular and biochemical abnormalities, capable of forming a systemic disease, for example forming tumor metastasis in distant organs.

Various diseases are caused by aberrant cell proliferation (“hyperproliferation”) as well as evasion from apoptosis. These diseases include e.g. benign hyperplasia like that of the prostate (“BPH”) or colon epithelium, psoriasias, glomerulonephritis or osteoarthritis. Most importantly these diseases include malignant neoplasia commonly described as cancer and characterized by tumor cells finally metastasizing into distinct organs or tissues. Malignant neoplasia include solid and hematological tumors. Solid tumors are exemplified by tumors of the breast, bladder, bone, brain, central and peripheral nervus system, colon, endocrine glands (eg thyroid and adrenal cortex), esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva. Malignant neoplasia include inherited cancers exemplified by retinoblastoma and Wilms tumor. In addition, malignant neoplasia include primary tumors in said organs and corresponding secondary tumors in distant organs (“tumor metastases”). Hematological tumors are exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, cancers of unknown primary site as well as AIDS related malignancies.

It is to be noted that a cancer disease as well as a malignant neoplasia does not necessarily require the formation of metastases in distant organs. Certain tumors exert devastating effects on the primary organ itself through their aggressive growth properties. These can lead to the destruction of the tissue and organ structure finally resulting in failure of the assigned organ function.

Neoplastic cell proliferation might affect normal cell behaviour and organ function. For example the formation of new blood vessels, a process described as neovascularization, is induced by tumors or tumor metastases. Compounds according to this invention can be commercially applicable for treatment of pathophysiological relevant processes caused by benign or neoplastic cell proliferation, such as but not limited to neovascularization by unphysiological proliferation of vascular endothelial cells.

Drug resistance is of particular importance for the frequent failure of standard cancer therapeutics. This drug resistance is caused by various cellular and molelcular mechanisms like overexpression of drug efflux pumps or mutation within the cellular target protein. The commercial applicability of the compounds according to this invention is not limited to 1^{st} line treatment of patients. Patients with resistance to defined cancer chemotherapeutics or target specific anti-cancer drugs (2^{nd} or 3^{rd} line treatment) can be also amenable for treatment with the compounds according to this invention.

The compounds according to the present invention display a cell cycle dependent cytotoxic activity, more precisely a mitosis confined activity, leading to a mitotic arrest which inevitably results in the onset of apoptosis and/or cell death.

Compounds of the present invention induce a strongly increased phosphorylation of histone H3 when incubated with test cells for more than 8 hours and less than 48 hours at concentrations around the IC50 value of the cytotoxicity or above. Moreover, treatment of cells with compounds of this invention does not induce polyploidy or multinuclearity as primary mode of action.

Compounds according to the present invention can be commercially applicable for treatment, prevention or amelioration of the diseases of benign and malignant behavior as described before, such as e.g. benign or malignant neoplasia, particularly cancer, such as e.g. any of those cancer diseases described above.

Accordingly, the invention relates to compounds according to the invention or pharmaceutically acceptable salts thereof for the treatment of (hyper)proliferative diseases and/or disorders responsive to the induction of apoptosis. The invention further relates to a pharmaceutical composition, comprising a compound according to the invention or a pharmaceutically acceptable salt thereof, for the treatment of (hyper)proliferative diseases and/or disorders responsive to the induction of apoptosis.

In the context of their properties, functions and usabilities mentioned herein, the compounds according to the present invention are expected to be distinguished by valuable and desirable effects related therewith, such as e.g. by low toxicity, superior bioavailability in general (such as e.g. good enteral absorption), superior therapeutic window, absence of significant side effects, and/or further beneficial effects related with their therapeutic and pharmaceutical suitability.

The invention further includes a method for treating (hyper)proliferative diseases and/or disorders responsive to the induction of apoptosis, particularly those diseases, disorders, conditions or illnesses mentioned above, in mammals, including humans, suffering therefrom comprising administering to said mammals in need thereof a pharmacologically active and therapeutically effective and tolerable amount of one or more of the compounds according to this invention.

The present invention further includes a method useful to modulate apoptosis and/or aberrant cell growth in the therapy of benign or malignant neoplastic diseases, such as e.g. cancer, comprising administering to a subject in need of such therapy a therapeutically active and pharmacologically effective and tolerable amount of one or more of the compounds according to this invention.

The present invention further relates to the use of the compounds according to this invention for the production of pharmaceutical compositions which are employed for the treatment, prophylaxis and/or amelioration of the illnesses mentioned.

The present invention further relates to the use of the compounds according to this invention for the production of pharmaceutical compositions which can be used in the treatment, prevention or amelioration of (hyper)proliferative diseases of benign or malignant behaviour and/or disorders responsive to the induction of apoptosis in a mammal, such as, for example, benign or malignant neoplasia, e.g. cancer.

The present invention further relates to the use of the compounds according to this invention for the production of pharmaceutical compositions which can be used use in the treatment, prevention or amelioration of disorders responsive to arresting of aberrant cell growth and/or induction of apoptosis.

The present invention further relates to the use of the compounds according to this invention for the production of pharmaceutical compositions for treating, preventing or ameliorating benign or malignant neoplasia, particularly cancer, such as e.g. any of those cancer diseases described above.

The present invention further relates to pharmaceutical compositions comprising one or more of the compounds according to this invention and a pharmaceutically acceptable carrier or diluent.

The present invention further relates to pharmaceutical compositions made by combining one or more of the compounds according to this invention and a pharmaceutically acceptable carrier or diluent.

The present invention further relates to pharmaceutical compositions comprising one or more of the compounds according to this invention and pharmaceutically acceptable auxiliaries and/or excipients.

The present invention further relates to combinations comprising one or more compounds according to this invention and pharmaceutically acceptable auxiliaries, excipients and/or vehicles, e.g. for treating, preventing or ameliorating benign or malignant neoplasia, particularly cancer, such as e.g. any of those cancer diseases described above.

The present invention further relates to a combination comprising a compound according to this invention and a pharmaceutically acceptable excipient, carrier and/or diluent, e.g. for treating, preventing or ameliorating benign or malignant neoplasia, particularly cancer, such as e.g. any of those cancer diseases described above.

The present invention further relates to a composition consisting essentially of a therapeutically effective and tolerable amount of one or more compounds according to this invention together with the usual pharmaceutically acceptable vehicles, diluents and/or excipients for use in therapy, e.g. for treating, preventing or ameliorating hyperproliferative diseases, such as e.g. cancer, and/or disorders responsive to induction of apoptosis.

The present invention further relates to compounds according to this invention for use in therapy, such as, for example, in the treatment, prevention or amelioration (hyper)proliferative diseases of benign or malignant behaviour and/or disorders responsive to the induction of apoptosis, such as e.g. those diseases mentioned herein, particularly cancer.

The present invention further relates to compounds according to this invention having anti-proliferative and/or apoptosis inducing activity.

The present invention further relates to pharmaceutical compositions according to this invention having anti-proliferative activity.

The present invention further relates to pharmaceutical compositions according to this invention having apoptosis inducing activity.

The invention further relates to the use of a pharmaceutical composition comprising one or more of the compounds according to this invention as sole active ingredient(s) and a pharmaceutically acceptable carrier or diluent in the manufacture of pharmaceutical products for the treatment and/or prophylaxis of the illnesses mentioned above.

Additionally, the invention relates to an article of manufacture, which comprises packaging material and a pharmaceutical agent contained within said packaging material, wherein the pharmaceutical agent is therapeutically effective inhibiting cellular (hyper)proliferation and/or inducing apoptosis, ameliorating the symptoms of a (hyper)proliferative disease and/or a disorder responsive to the induction of apoptosis, and wherein the packaging material comprises a label or package insert which indicates that the pharmaceutical agent is useful for treating, preventing or ameliorating a (hyper)proliferative disease and/or a disorder responsive to the induction of apoptosis, and wherein said pharmaceutical agent comprises one or more compounds according to the invention. The packaging material, label and package insert otherwise parallel or resemble what is generally regarded as standard packaging material, labels and package inserts for pharmaceuticals having related utilities.

The pharmaceutical compositions according to this invention are prepared by processes which are known per se and familiar to the person skilled in the art. As pharmaceutical compositions, the compounds of the invention (=active compounds) are either employed as such, or preferably in combination with suitable pharmaceutical auxiliaries and/or excipients, e.g. in the form of tablets, coated tablets, dragees, pills, cachets, granules, capsules, caplets, suppositories, patches (e.g. as TTS), emulsions (such as e.g. micro-emulsions or lipid emulsions), suspensions (such as e.g. nano suspensions), gels, solubilisates or solutions (e.g. sterile solutions), or encapsuled in liposomes or as beta-cyclodextrine or beta-cyclodextrine derivative inclusion complexes or the like, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other active compound excipients, for example antioxidants, dispersants, emulsifiers, preservatives, solubilizers (such as e.g. polyoxyethylenglyceroltriricinoleat 35, PEG 400, Tween 80, Captisol, Solutol HS15 or the like), colorants, complexing agents, permeation promoters, stabilizers, fillers, binders, thickeners, disintegrating agents, buffers, pH regulators (e.g. to obtain neutral, alkaline or acidic formulations), polymers, lubricants, coating agents, propellants, tonicity adjusting agents, surfactants, flavorings, sweeteners or dyes, can be used.

In particular, auxiliaries and/or excipients of a type appropriate to the desired formulation and the desired mode of administration are used.

The administration of the compounds, pharmaceutical compositions or combinations according to the invention may be performed in any of the generally accepted modes of administration available in the art. Illustrative examples of suitable modes of administration include intravenous, oral, nasal, parente-ral, topical, transdermal and rectal delivery. Oral and intravenous delivery are preferred.

For the treatment of dermatoses, the compounds of the invention can be in particular administered in the form of those pharmaceutical compositions which are suitable for topical application. For the production of the pharmaceutical compositions, the compounds of the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, lotions, pastes, gels or solutions.

The pharmaceutical compositions according to the invention are prepared by processes known per se. The dosage of the compounds of the invention (=active compounds) is carried out in the order of magnitude customary for inhibitors of cellular (hyper)proliferation or apoptosis inducers. Topical application forms (such as ointments) for the treatment of dermatoses thus contain the active compounds in a concentration of, for example, 0.1-99%. The customary dose in the case of systemic therapy (p.o.) may be between 0.03 and 60 mg/kg per day, (i. v.) may be between 0.03 and 60 mg/kg/h. In another embodiment, the customary dose in the case of systemic therapy (p.o.) is between 0.3 and 30 mg/kg per day, (i. v.) is between 0.3 and 30 mg/kg/h.

The choice of the optimal dosage regime and duration of medication, particularly the optimal dose and manner of administration of the active compounds necessary in each case can be determined by a person skilled in the art on the basis of his/her expert knowledge.

Depending upon the particular disease, to be treated or prevented, additional therapeutic active agents, which are normally administered to treat or prevent that disease, may optionally be coadministered with the compounds according to this invention. As used herein, additional therapeutic agents that are normally administered to treat or prevent a particular disease are known as appropriate for the disease being treated.

For example, compounds according to this invention may be combined with one or more standard therapeutic agents used for treatment of the diseases as mentioned before.

In one particular embodiment, compounds according to this invention may be combined with one or more art-known anti-cancer agents, such as e.g. with one or more chemotherapeutic and/or target specific anti-cancer agents as described below.

Examples of known chemotherapeutic anti-cancer agents frequently used in combination therapy include, but not are limited to (i) alkylating/carbamylating agents such as Cyclophosphamid (Endoxan®), Ifosfamid (Holoxan®), Thiotepa (Thiotepa Lederle®), Melphalan (Alkeran®), or chloroethylnitrosourea (BCNU); (ii) platinum derivatives like cis-platin (Platinex® BMS), oxaliplatin, satraplatin or carboplatin (Cabroplat® BMS); (iii) antimitotic agents/tubulin inhibitors such as vinca alkaloids (vincristine, vinblastine, vinorelbine), taxanes such as Paclitaxel (Taxol®), Docetaxel (Taxotere®) and analogs as well as new formulations and conjugates thereof, epothilones such as Epothilone B (Patupilone®), Azaepothilone (Ixabepilone®) or ZK-EPO, a fully synthetic epothilone B analog; (iv) topoisomerase inhibitors such as anthracyclines (exemplified by Doxorubicin/Adriblastin®), epipodophyllotoxines (examplified by Etoposide/Etopophos®) and camptothecin and camptothecin analogs (exemplified by Irinotecan/Camptosar® or Topotecan/Hycamtin®); (v) pyrimidine antagonists such as 5-fluorouracil (5-FU), Capecitabine (Xeloda®), Arabinosylcytosine/Cytarabin (Alexan®) or Gemcitabine (Gemzar®); (vi) purin antagonists such as 6-mercaptopurine (Puri-Nethol®), 6-thioguanine or fludarabine (Fludara®) and finally (vii) folic acid antagonists such as methotrexate (Farmitrexat®) or premetrexed (Alimta®).

Examples of target specific anti-cancer drug classes used in experimental or standard cancer therapy include but are not limited to (i) kinase inhibitors such as e.g. Imatinib (Glivec®), ZD-1839/Gefitinib (Iressa®), Bay43-9006 (Sorafenib, Nexavar®), SU11248/Sunitinib (Sutent®) or OSI-774/Erlotinib (Tarceva®), Dasatinib (Sprycel®), Lapatinib (Tykerb®), or, see also below, Vatalanib, Vandetanib (Zactima®) or Pazopanib; (ii) proteasome inhibitors such as PS-341/Bortezumib (Velcade®); (iii) histone deacetylase inhibitors like SAHA, PXD101, MS275, MGCD0103, Depsipeptide/FK228, NVP-LBH589, NVP-LAQ824, Valproic acid (VPA) and butyrates (iv) heat shock protein 90 inhibitors like 17-allylaminogeldanamycin (17-AAG); (v) vascular targeting agents (VTAs) like combretastin A4 phosphate or AVE8062/AC7700 and anti-angiogenic drugs like the VEGF antibodies, such as Bevacizumab (Avastin®), or KDR tyrosine kinase inhibitors such as PTK787/ZK222584 (Vatalanib) or Vandetanib (Zactima®) or Pazopanib; (vi) monoclonal antibodies such as Trastuzumab (Herceptin®) or Rituximab (MabThera/Rituxan®) or Alemtuzumab (Campath®) or Tositumomab (Bexxar®) or C225/Cetuximab (Erbitux®) or Avastin (see above) or Panitumumab as well as mutants and conjugates of monoclonal antibodies, e.g. Gemtuzumab ozogamicin (Mylotarg®) or Ibritumomab tiuxetan (Zevalin®), and antibody fragments; (vii) oligonucleotide based therapeutics like G-3139/Oblimersen (Genasense®); (viii) Toll-like receptor/TLR 9 agonists like Promune®, TLR 7 agonists like Imiquimod (Aldara®) or Isatoribine and analogues thereof, or TLR 7/8 agonists like Resiquimod as well as immunostimulatory RNA as TLR 7/8 agonists; (ix) protease inhibitors (x) hormonal therapeutics such as anti-estrogens (e.g. Tamoxifen or Raloxifen), anti-androgens (e.g. Flutamide or Casodex), LHRH analogs (e.g. Leuprolide, Goserelin or Triptorelin) and aromatase inhibitors.

Other known target specific anti-cancer agents which may be used for combination therapy include bleomycin, retinoids such as all-trans retinoic acid (ATRA), DNA methyltransferase inhibitors such as the 2-deoxycytidine derivative Decitabine (Docagen®) and 5-Azacytidine, alanosine, cytokines such as interleukin-2, interferons such as interferon α2 or interferon-γ, death receptor agonists, such as TRAIL, DR4/5 agonistic antibodies, FasL and TNF-R agonists (e.g. TRAIL receptor agonists like mapatumumab or lexatumumab).

As exemplary anti-cancer agents, which may be useful in the combination therapy according to the present invention, any of the following drugs may be mentioned, without being restricted thereto, 5 FU, actinomycin D, ABARELIX, ABCIXIMAB, ACLARUBICIN, ADAPALENE, ALEMTUZUMAB, ALTRETAMINE, AMINOGLUTETHIMIDE, AMIPRILOSE, AMRUBICIN, ANASTROZOLE, ANCITABINE, ARTEMISININ, AZATHIOPRINE, BASILIXIMAB, BENDAMUSTINE, BEVACIZUMAB, BEXXAR, BICALUTAMIDE, BLEOMYCIN, BORTEZOMIB, BROXURIDINE, BUSULFAN, CAMPATH, CAPECITABINE, CARBOPLATIN, CARBOQUONE, CARMUSTINE, CETRORELIX, CHLORAMBUCIL, CHLORMETHINE, CISPLATIN, CLADRIBINE, CLOMIFENE, CYCLOPHOSPHAMIDE, DACARBAZINE, DACLIZUMAB, DACTINOMYCIN, DASATINIB, DAUNORUBICIN, DECITABINE, DESLORELIN, DEXRAZOXANE, DOCETAXEL, DOXIFLURIDINE, DOXORUBICIN, DROLOXIFENE, DROSTANOLONE, EDELFOSINE, EFLORNITHINE, EMITEFUR, EPIRUBICIN, EPITIOSTANOL, EPTAPLATIN, ERBITUX, ERLOTINIB, ESTRAMUSTINE, ETOPOSIDE, EXEMESTANE, FADROZOLE, FINASTERIDE, FLOXURIDINE, FLUCYTOSINE, FLUDARABINE, FLUOROURACIL, FLUTAMIDE, FORMESTANE, FOSCARNET, FOSFESTROL, FOTEMUSTINE, FULVESTRANT, GEFITINIB, GENASENSE, GEMCITABINE, GLIVEC, GOSERELIN, GUSPERIMUS, HERCEPTIN, IDARUBICIN, IDOXURIDINE, IFOSFAMIDE, IMATINIB, IMPROSULFAN, INFLIXIMAB, IRINOTECAN, IXABEPILONE, LANREOTIDE, LAPATINIB, LETROZOLE, LEUPRORELIN, LOBAPLATIN, LOMUSTINE, LUPROLIDE, MELPHALAN, MERCAPTOPURINE, METHOTREXATE, METUREDEPA, MIBOPLATIN, MIFEPRISTONE, MILTEFOSINE, MIRIMOSTIM, MITOGUAZONE, MITOLACTOL, MITOMYCIN, MITOXANTRONE, MIZORIBINE, MOTEXAFIN, MYLOTARG, NARTOGRASTIM, NEBAZUMAB, NEDAPLATIN, NILUTAMIDE, NIMUSTINE, OCTREOTIDE, ORMELOXIFENE, OXALIPLATIN, PACLITAXEL, PALIVIZUMAB, PANITUMUMAB, PATUPILONE, PAZOPANIB, PEGASPARGASE, PEGFILGRASTIM, PEMETREXED, PENTETREOTIDE, PENTOSTATIN, PERFOSFAMIDE, PIPOSULFAN, PIRARUBICIN, PLICAMYCIN, PREDNIMUSTINE, PROCARBAZINE, PROPAGERMANIUM, PROSPIDIUM CHLORIDE, RALOXIFEN, RALTITREXED, RANIMUSTINE, RANPIRNASE, RASBURICASE, RAZOXANE, RITUXIMAB, RIFAMPICIN, RITROSULFAN, ROMURTIDE, RUBOXISTAURIN, SARGRAMOSTIM, SATRAPLATIN, SIROLIMUS, SOBUZOXANE, SORAFENIB, SPIROMUSTINE, STREPTOZOCIN, SUNITINIB, TAMOXIFEN, TASONERMIN, TEGAFUR, TEMOPORFIN, TEMOZOLOMIDE, TENIPOSIDE, TESTOLACTONE, THIOTEPA, THYMALFASIN, TIAMIPRINE, TOPOTECAN, TOREMIFENE, TRAIL, TRASTUZUMAB, TREOSULFAN, TRIAZIQUONE, TRIMETREXATE, TRIPTORELIN, TROFOSFAMIDE, UREDEPA, VALRUBICIN, VATALANIB, VANDETANIB, VERTEPORFIN, VINBLASTINE, VINCRISTINE, VINDESINE, VINORELBINE, VOROZOLE and ZEVALIN.

The anti-cancer agents mentioned herein above as combination partners of the compounds according to this invention are meant to include pharmaceutically acceptable derivatives thereof, such as e.g. their pharmaceutically acceptable salts.

The person skilled in the art is aware on the base of his/her expert knowledge of the kind, total daily dosage(s) and administration form(s) of the additional therapeutic agent(s) coadministered. Said total daily dosage(s) can vary within a wide range.

In practicing the present invention, the compounds according to this invention may be administered in combination therapy separately, sequentially, simultaneously, concurrently or chronologically staggered (such as e.g. as combined unit dosage forms, as separate unit dosage forms, as adjacent discrete unit dosage forms, as fixed or non-fixed combinations, as kit-of-parts or as admixtures) with one or more standard therapeutics, in particular art-known anti-cancer agents (chemotherapeutic and/or target specific anti-cancer agents), such as e.g. any of those mentioned above.

In this context, the present invention further relates to a combination comprising

a first active ingredient, which is at least one compound according to this invention, and
a second active ingredient, which is at least one art-known anti-cancer agent, such as e.g. one or more of those mentioned herein above,
for separate, sequential, simultaneous, concurrent or chronologically staggered use in therapy, such as e.g. in therapy of any of those diseases mentioned herein.

The term “combination” according to this invention may be present as a fixed combination, a non-fixed combination or a kit-of-parts.

A “fixed combination” is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously, concurrently or chronologically staggered.

Sequential administration encompasses a short time period between the administration of components (A), (B) and optionally (C) of the combination product or the kit-of-parts according to the invention (for example, the time that is needed to swallow one tablet after the other).

Separate administration encompasses both short and long time periods between the administration of components (A), (B) and optionally (C) of the combination product or the kit-of-parts according to the invention. However, for the purposes of the present invention at least one of the components is administered while the other component(s) is (are) still having an effect on the patient being treated. In a preferred embodiment of the invention the effect on the patient being treated is a synergistic effect.

The combined administration of compound (A) or a pharmaceutically acceptable salt thereof and one or two other active compound(s) or pharmaceutically acceptable salt(s) thereof which is (are) used in the treatment of (hyper)proliferative diseases, particularly cancer, either in form of the pharmaceutical composition, combination product or kit-of-parts according to the invention, lead to an effective treatment of (hyper)proliferative diseases, particularly cancer, and in a preferred embodiment is superior to the use of either active agent alone. Moreover, in a particularly preferred embodiment, the combined administration of compound (A) or a pharmaceutically acceptable salt thereof and one or two other active compound(s) or pharmaceutically acceptable salt(s) thereof which is (are) used in the treatment of (hyper)proliferative diseases, particularly cancer, shows a synergistic efficacy for treating (hyper)proliferative diseases.

As used herein, the term “synergistic” refers to the combination of compound (A) or a pharmaceutically acceptable salt thereof with one or two other active compound(s) or pharmaceutically acceptable salt(s) thereof which is (are) used in the treatment of (hyper)proliferative diseases, particularly cancer, either in form of the pharmaceutical composition, combination product or kit-of-parts according to the invention having an efficacy for the treatment of (hyper)proliferative diseases that is greater than would be expected from the sum of their individuals effects. The synergistic effects of the embodiments of the present invention encompass additional unexpected advantages for the treatment of (hyper)proliferative diseases, particularly cancer. Such additional advantages may include, but are not limited to, lowering the required dose of one or more of the active agents of the combination, reducing the side effects of one or more of the active agents of the combination, or rendering one or more of the active agents more tolerable to the patient in need of a (hyper)proliferative disease therapy. The combined administration of compound (A) or a pharmaceutically acceptable salt thereof and one or two other active compound(s) or pharmaceutically acceptable salts thereof which is (are) used in the treatment of (hyper)proliferative diseases may also be useful for decreasing the required number of separate dosages, thus, potentially improving compliance of the patient in need of (hyper)proliferative diseases therapy.

The present invention further relates to a pharmaceutical composition comprising

a first active ingredient, which is at least one compound according to this invention, and
a second active ingredient, which is at least one art-known anti-cancer agent, such as e.g. one or more of those mentioned herein above, and, optionally,
a pharmaceutically acceptable carrier or diluent,
for separate, sequential, simultaneous, concurrent or chronologically staggered use in therapy.

The present invention further relates to a combination product comprising

a.) at least one compound according to this invention formulated with a pharmaceutically acceptable carrier or diluent, and
b.) at least one art-known anti-cancer agent, such as e.g. one or more of those mentioned herein above, formulated with a pharmaceutically acceptable carrier or diluent.

The present invention further relates to a kit-of-parts comprising a preparation of a first active ingredient, which is a compound according to this invention, and a pharmaceutically acceptable carrier or diluent; a preparation of a second active ingredient, which is an art-known anti-cancer agent, such as one of those mentioned above, and a pharmaceutically acceptable carrier or diluent; for simultaneous, concurrent, sequential, separate or chronologically staggered use in therapy. Optionally, said kit comprises instructions for its use in therapy, e.g. to treat (hyper)proliferative diseases and/or disorders responsive to the induction of apoptosis, such as e.g. cancer, more precisely, any of those cancer diseases described above.

The present invention further relates to a combined preparation comprising at least one compound according to this invention and at least one art-known anti-cancer agent for simultaneous, concurrent, sequential or separate administration.

In this connection, the present invention further relates to combinations, compositions, formulations, preparations or kits according to the present invention having anti-proliferative and/or apoptosis inducing properties.

In addition, the present invention further relates to a method for treating in combination therapy (hyper)proliferative diseases and/or disorders responsive to the induction of apoptosis, such as e.g. cancer, in a patient comprising administering a combination, composition, formulation, preparation or kit as described herein to said patient in need thereof.

In addition, the present invention further relates to a method for treating (hyper)proliferative diseases of benign or malignant behaviour and/or disorders responsive to the induction of apoptosis, such as e.g. cancer, in a patient comprising administering in combination therapy separately, simultaneously, concurrently, sequentially or chronologically staggered a pharmaceutically active and therapeutically effective and tolerable amount of a pharmaceutical composition, which comprises a compound according to this invention and a pharmaceutically acceptable carrier or diluent, and a Pharmaceutically active and therapeutically effective and tolerable amount of one or more art-known anti-cancer agents, such as e.g. one or more of those mentioned herein, to said patient in need thereof.

In further addition, the present invention relates to a method for treating, preventing or ameliorating (hyper)proliferative diseases and/or disorders responsive to induction of apoptosis, such as e.g. benign or malignant neoplasia, e.g. cancer, particularly any of those cancer diseases mentioned herein, in a patient comprising administering separately, simultaneously, concurrently, sequentially or chronologically staggered to said patient in need thereof an amount of a first active compound, which is a compound according to the present invention, and an amount of at least one second active compound, said at least one second active compound being a standard therapeutic agent, particularly at least one art-known anti-cancer agent, such as e.g. one or more of those chemotherapeutic and target-specific anti-cancer agents mentioned herein, wherein the amounts of the first active compound and said second active compound result in a therapeutic effect.

In yet further addition, the present invention relates to a method for treating, preventing or ameliorating (hyper)proliferative diseases and/or disorders responsive to induction of apoptosis, such as e.g. benign or malignant neoplasia, e.g. cancer, particularly any of those cancer diseases mentioned herein, in a patient comprising administering a combination according to the present invention.

In addition, the present invention further relates to the use of a composition, combination, formulation, preparation or kit according to this invention in the manufacture of a pharmaceutical product, such as e.g. a commercial package or a medicament, for treating, preventing, or ameliorating (hyper)proliferative diseases, such as e.g. cancer, and/or disorders responsive to the induction of apoptosis, particularly those diseases mentioned herein, such as e.g. malignant or benign neoplasia.

The present invention further relates to a commercial package comprising one or more compounds of the present invention together with instructions for simultaneous, concurrent, sequential or separate use with one or more chemotherapeutic and/or target specific anti-cancer agents, such as e.g. any of those mentioned herein.

The present invention further relates to a commercial package consisting essentially of one or more compounds of the present invention as sole active ingredient together with instructions for simultaneous, concurrent, sequential or separate use with one or more chemotherapeutic and/or target specific anti-cancer agents, such as e.g. any of those mentioned herein.

The present invention further relates to a commercial package comprising one or more chemotherapeutic and/or target specific anti-cancer agents, such as e.g. any of those mentioned herein, together with instructions for simultaneous, concurrent, sequential or separate use with one or more compounds according to the present invention.

The compositions, combinations, preparations, formulations, kits or packages mentioned in the context of the combination therapy according to this invention may also include more than one of the compounds according to this invention and/or more than one of the art-known anti-cancer agents mentioned.

The first and second active ingredient of a combination or kit-of-parts according to this invention may be provided as separate formulations (i.e. independently of one another), which are subsequently brought together for simultaneous, concurrent, sequential, separate or chronologically staggered use in combination therapy; or packaged and presented together as separate components of a combination pack for simultaneous, concurrent, sequential, separate or chronologically staggered use in combination therapy.

The type of pharmaceutical formulation of the first and second active ingredient of a combination or kit-of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. intravenous administration.

The amounts of the first and second active ingredients of the combinations, compositions or kits according to this invention may together comprise a therapeutically effective amount for the treatment, prophylaxis or amelioration of a (hyper)proliferative diseases and/or a disorder responsive to the induction of apoptosis, particularly one of those diseases mentioned herein, e.g. benign or malignant neoplasia, especially cancer, like any of those cancer diseases mentioned herein.

In addition, compounds according to the present invention can be used in the pre- or post-surgical treatment of cancer.

In further addition, compounds of the present invention can be used in combination with radiation therapy.

A combination according to this invention can refer to a composition comprising both the compound(s) according to this invention and the other active anti-cancer agent(s) in a fixed combination (fixed unit dosage form), or a medicament pack comprising the two or more active ingredients as discrete separate dosage forms (non-fixed combination). In case of a medicament pack comprising the two or more active ingredients, the active ingredients are preferably packed into blister cards which are suited for improving compliance.

Each blister card preferably contains the medicaments to be taken on one day of treatment. If the medicaments are to be taken at different times of day, the medicaments can be disposed in different sections on the blister card according to the different ranges of times of day at which the medicaments are to be taken (for example morning and evening or morning, midday and evening). The blister cavities for the medicaments to be taken together at a particular time of day are accommodated in the respective range of times of day. The various times of day are, of course, also put on the blister in a clearly visible way. It is also possible, of course, for example to indicate a period in which the medicaments are to be taken, for example stating the times.

The daily sections may represent one line of the blister card, and the times of day are then identified in chronological sequence in this column.

Medicaments which must be taken together at a particular time of day are placed together at the appropriate time on the blister card, preferably a narrow distance apart, allowing them to be pushed out of the blister easily, and having the effect that removal of the dosage form from the blister is not forgotten.

### Biological Investigations

The anti-proliferative/cytotoxic activity of the compounds described herein, can be tested on subclones of RKO (RKOp27) human colon adenocarcinoma cells (Schmidt et al., Oncogene 19, 2423-2429; 2000) using the Alamar Blue cell viability assay (described in O'Brien et al. Eur J Biochem 267, 5421-5426, 2000). The compounds are dissolved as 20 mM solutions in dimethylsulfoxide (DMSO) and subsequently diluted in semi-logarithmic steps. DMSO dilutions are further diluted 1:100 into Dulbecco's modified Eagle's medium (DMEM) containing 10% fetal calf serum to a final concentration twice as much as the final concentration in the test. RKO subclones are seeded into 96 well flat bottom plates at a density of 5000 cells per well in a volume of 50 μl per well. 24 hours after seeding the 50 μl each of the compound dilutions in DMEM medium are added into each well of the 96 Well plate. Each compound dilution is tested as quadruplicates. Wells containing untreated control cells are filled with 50 μl DMEM medium containing 1% DMSO. The cells are then incubated with the substances for 72 hours at 37° C. in a hunified atmosphere containing 5% carbon dioxide. To determine the viability of the cells, 10 μl of an Alamar Blue solution (Biosource) are added and the fluorescence is measured at an extinction of 544 nm and an emission of 590 nm. For the calculation of the cell viability the emission value from untreated cells is set as 100% viability and the emission rates of treated cells are set in relation to the values of untreated cells. Viabilities are expressed as % values.

The corresponding IC₅₀ values of the compounds for anti-proliferative/cytotoxic activity are determined from the concentration-effect curves.

To determine the cell cycle specific mode of action, subclones of RKO colon adenocarcinoma cells (RKOp27 or RKOp21 as described by Schmidt et al. in Oncogene 19, 2423-2429; 2000) are seeded into 96 well flat bottom plates at a density of 15000 cells per well in a volume of 50 μl per well in DMEM growth medium with 10% FCS containing 10 μM Ponasterone A. 24 hours after seeding the 50 μl each of the compound dilutions in DMEM medium are added into each well of the 96 Well plate. Each compound dilution is tested as quadruplicates. Wells containing untreated control cells are filled with 50 μl DMEM medium containing 1% DMSO. The cells are then incubated with the substances for 72 hours at 37° C. in a humidified athmosphere containing 5% carbon dioxide. To determine the viability of the cells, 10 μl of an Alamar Blue solution (Biosource) are added and the fluorescence was measured at an extinction of 544 nm and an emission of 590 nm. For the calculation of the cell viability the emission value from untreated cells is set as 100% viability and the emission rates of treated cells are set in relation to the values of untreated cells. Viabilities are expressed as % values. Viability is compared of proliferating cells grown in the absence of the inducer Ponasterone A, versus viability of cells arrested by the expression of ectopic p27Kip1 induced by Ponasterone A.

Representative IC₅₀ values for anti-proliferation/cytotoxicity determined in the mentioned assays follow from the following table A, in which the numbers of the compound correspond to the numbers of the examples.

TABLE A
Anti-proliferative/cytotoxic activity

| | IC₅₀ RKO p27 induced | IC₅₀ RKO p27 induced |
|---|---|---|
| | (arrested) ≧100 μM | (arrested) ≧60 μM |
| IC₅₀ RKO p27 | 3, 13, 14, 15, 16, 18, | 1, 19, 20, 29, 37, 46 |
| uninduced | 21, 22, 25, 28, 31, 33, | 52, 101, 111, 117 |
| (proliferating) ≦0.5 μM | 34, 43, 48, 51, 54, 58, | |
| | 60, 61, 70, 76, 77, 78, | |
| | 80, 84, 85, 88, 89, 96, | |
| | 97, 98, 99, 105 to 110, | |
| | 112 to 116, 118 | |
| IC₅₀ RKO p27 | 7, 23, 27, 36, 39, 41, | 24, 62 |
| uninduced | 42, 45, 50, 59, 65, 71, | |
| (proliferating) >0.5 μM | 72, 79, 104 | |
| but ≦2 μM | | |

To test the anti-proliferative activity/cytotoxicity on cells known to be highly resistant towards distinct classes of chemotherapeutics, HCT15 cells (with P-glycoprotein overexpression) and MCF7 ADR cells, both of them are known to overexpress certain classes of multidrug resistance transporters are used in Alamar Blue assays as described above. Briefly, the compounds are dissolved as 20 mM solutions in dimethylsulfoxide (DMSO) and subsequently diluted in semi-logarithmic steps. DMSO dilutions were further diluted 1:100 into Dulbecco's modified Eagle's medium (DMEM) containing 10% fetal calf serum to a final concentration twice as much as the final concentration in the test. The cells to be tested are seeded into 96 well flat bottom plates at a density of 10000 cells per well in a volume of 50 μl per well. 24 hours after seeding the 50 μl each of the compound dilutions in DMEM medium are added into each well of the 96 Well plate. Each compound dilution is tested as quadruplicates. Wells containing untreated control cells are filled with 50 μl DMEM medium containing 1% DMSO. The cells are then incubated with the substances for 72 hours at 37° C. in a humidified athmosphere containing 5% carbon dioxide. To determine the viability of the cells, 10 μl of an Alamar Blue solution (Biosource) are added and the fluorescence was measured at an extinction of 544 nm and an emission of 590 nm. For the calculation of the cell viability the emission value from untreated cells is set as 100% viability and the emission rates of treated cells are set in relation to the values of untreated cells. Viabilities are expressed as % values.

The induction of apoptosis can be measured by using a Cell death detection ELISA (Roche Biochemicals, Mannheim, Germany). RKO subclones are seeded into 96 well flat bottom plates at a density of 10000 cells per well in a volume of 50 μl per well. 24 hours after seeding the 50 μl each of the compound dilutions in DMEM medium are added into each well of the 96 Well plate. Each compound dilution is tested at least as triplicates. Wells containing untreated control cells are filled with 50 μl DMEM medium containing the same amount of DMSO as wells treated with compounds. The cells are then incubated with the substances for 24 hours at 37° C. in a humidified athmosphere containing 5% carbon dioxide. As a positive control for the induction of apoptosis, cells are treated with 50 μM Cisplatin (Gry Pharmaceuticals, Kirchzarten, Germany). Medium is then removed and the cells are lysed in 200 μl lysis buffer. After centrifugation as described by the manufacturer, 10 μl of cell lysate is processed as described in the protocol. The degree of apoptosis is calculated as follows: The absorbance at 405 nm obtained with lysates from cells treated with 50 μM cisplatin is set as 100 cpu (cisplatin units), while an absorbance at 405 nm of 0.0 was set as 0.0 cpu. The degree of apoptosis is expressed as cpu in relation to the value of 100 cpu reached with the lysates obtained from cells treated with 50 μM cisplatin.

1. Compounds of formula I 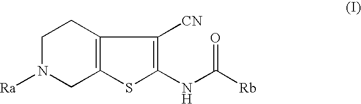
wherein
Ra is —C(O)—N(R11)-R1, in which
R1 is 1-4C-alkyl, 3-7C-cycloalkyl, HetA, phenyl, HarA, 1-4C-alkyl substituted by Raa, or 2-4C-alkyl substituted by Rab and Rac on different carbon atoms,
wherein said 3-7C-cycloalkyl may be optionally substituted by one or two substituents independently selected from R12, and
wherein each of said phenyl and HarA may be optionally substituted by one, two or three substituents independently selected from R13,
R11 is hydrogen, 1-4C-alkyl, 3-7C-cycloalkyl or 3-7C-cycloalkyl-1-4C-alkyl,
or R1 and R11 together and with inclusion of the nitrogen atom, to which they are attached, form a heterocyclic radical HET, in which
either
HET is optionally substituted by one or two substituents independently selected from R12, and is piperidin-1-yl, pyrrolidin-1-yl, morpholin-4-yl, thiomorpholin-4-yl or 4N-(1-4C-alkylcarbonyl)-piperazin-1-yl,
or
HET is optionally substituted by one or two substituents independently selected from R13, and is pyrrol-1-yl, imidazol-1-yl, pyrazol-1-yl or triazol-1-yl,
Rb is -T-Q, in which
T is a ethane-1,2-diyl, cyclopropane-1,2-diyl, or propane-1,2-diyl bridge, and
either
Q is optionally substituted by Rba and/or Rbb, and is phenyl,
or
Q is optionally substituted by Rca and/or Rcb, and is pyridyl,
or
Q is optionally substituted by Rda and/or Rdb, and is furyl or thienyl,
or
Q is optionally substituted by Rea and/or Reb, and is 3-7C-cycloalkyl,
wherein
Raa is selected from the group consisting of:
3-7C-cycloalkyl, phenyl,
halogen, trifluoromethyl, cyano, hydroxyl,
HarB, HetB, HetC, morpholino,
—C(O)R2, —C(O)OR3, —C(O)N(R4)R5,
—N(R4)R5, —N(R6)C(O)R7, —OC(O)R8,
completely or predominantly fluorine-substituted 1-4C-alkoxy, and
—OR9,
wherein said 3-7C-cycloalkyl may be optionally substituted by one or two substituents independently selected from R12, and
wherein each of said phenyl and HarB may be optionally substituted by one, two or three substituents independently selected from R13,
in which
R2, R3, R4, R5, R6, R7 and R8 may be the same or different and are independently selected from the group consisting of:
hydrogen and 1-4C-alkyl,
R9 is selected from the group consisting of:
1-4C-alkyl, 3-7C-cycloalkyl, 3-7C-cycloalkyl-1-4C-alkyl, hydroxy-2-4C-alkyl, 1-4C-alkoxy-2-4C-alkyl, phenyl-1-4C-alkyl, pyridyl-1-4C-alkyl, and (1-4C-alkoxy-2-4C-alkoxy)-2-4C-alkyl,
either
HarA is bonded to the parent molecular group via a ring carbon atom, and is a 5-membered monocyclic partially unsaturated or aromatic heterocyclic ring comprising one to four heteroatoms independently selected from nitrogen, oxygen and sulphur,
or
HarA is bonded to the parent molecular group via a ring carbon atom, and is a 6-membered monocyclic partially unsaturated or aromatic heterocyclic ring comprising one or two nitrogen atoms,
or
HarA is bonded to the parent molecular group via a ring carbon atom, and is a 5-membered monocyclic partially unsaturated or aromatic heterocyclic ring comprising one to three heteroatoms independently selected from nitrogen, oxygen and sulphur, which heterocyclic ring is substituted by one oxo group,
or
HarA is bonded to the parent molecular group via a ring carbon atom, and is a 6-membered monocyclic partially unsaturated or aromatic heterocyclic ring comprising one or two nitrogen atoms, which heterocyclic ring is substituted by one oxo group,
either
HarB is bonded to the parent molecular group via a ring carbon or a ring nitrogen atom, and is a 5-membered monocyclic partially unsaturated or aromatic heterocyclic ring comprising one to four heteroatoms independently selected from nitrogen, oxygen and sulphur,
or
HarB is bonded to the parent molecular group via a ring carbon atom, and is a 6-membered monocyclic partially unsaturated or aromatic heterocyclic ring comprising one or two nitrogen atoms,
or
HarB is bonded to the parent molecular group via a ring carbon or a ring nitrogen atom, and is a 5-membered monocyclic partially unsaturated or aromatic heterocyclic ring comprising one to three heteroatoms independently selected from nitrogen, oxygen and sulphur, which heterocyclic ring is substituted by one oxo group,
or
HarB is bonded to the parent molecular group via a ring carbon or a ring nitrogen atom, and is a 6-membered monocyclic partially unsaturated or aromatic heterocyclic ring comprising one or two nitrogen atoms, which heterocyclic ring is substituted by one oxo group,
each R12 may be the same or different and is independently selected from the group consisting of:
1-4C-alkyl, halogen, hydroxyl, and 1-4C-alkoxy,
each R13 may be the same or different and is independently selected from the group consisting of:
1-4C-alkyl, halogen, hydroxyl, 1-4C-alkoxy, amino, amino-1-4C-alkyl, mono- or di-1-4C-alkylamino, hydroxy-2-4C-alkoxy, 1-4C-alkoxy-2-4C-alkoxy, hydroxy-1-4C-alkyl, and 1-4C-alkoxy-1-4C-alkyl,
HetA is bonded to the parent molecular group via a ring carbon atom, and is tetrahydropyranyl, tetrahydrofuryl, 1N-(1-4C-alkylcarbonyl)-piperidinyl, 1N-(1-4C-alkylcarbonyl)-pyrrolidinyl, 1N-(formyl)-piperidinyl, 1N-(formyl)-pyrrolidinyl, tetrahydrothiapyranyl, tetrahydrothienyl, 1N—(R14)-piperidin-2-onyl, 1N—(R14)-pyrrolidin-2-onyl, tetrahydropyran-2-onyl, tetrahydrofuran-2-onyl, 3N—(R14)-oxazolidin-2-onyl, or 1N—(R14)-3N—(R15)-imidazolidin-2-onyl,
wherein each of said HetA may be optionally substituted by one or two substituents independently selected from R16,
HetB is bonded to the parent molecular group via a ring nitrogen atom, and is piperidin-2-on-1-yl, pyrrolidin-2-on-1-yl, oxazolidin-2-on-1-yl, or 3N—(R15)-imidazolidin-2-on-1-yl,
wherein each of said HetB may be optionally substituted by one or two substituents independently selected from R16,
HetC is bonded to the parent molecular group via a ring carbon atom, and is tetrahydropyranyl, tetrahydrofuryl, 1N-(1-4C-alkylcarbonyl)-piperidinyl, 1N-(1-4C-alkylcarbonyl)-pyrrolidinyl, 1N-(formyl)-piperidinyl, 1N-(formyl)-pyrrolidinyl, tetrahydrothiapyranyl, tetrahydrothienyl, 1N—(R14)-piperidin-2-onyl, 1N—(R14)-pyrrolidin-2-onyl, tetrahydropyran-2-onyl, tetrahydrofuran-2-onyl, 3N—(R14)-oxazolidin-2-onyl, or 1N—(R14)-3N—(R15)-imidazolidin-2-onyl,
wherein each of said HetC may be optionally substituted by one or two substituents independently selected from R16,
in which
R14 is hydrogen or 1-4C-alkyl,
R15 is hydrogen or 1-4C-alkyl,
each R16 may be the same or different and is independently selected from the group consisting of:
1-4C-alkyl, halogen, hydroxyl, and 1-4C-alkoxy,
Rab is hydroxyl,
Rac is hydroxyl,
or Rab and Rac bonded to adjacent carbon atoms form together an 1-2C-alkylenedioxy bridge which is optionally substituted by one or two substituents independently selected from fluorine and methyl,
or Rab and Rac bonded to carbon atoms two bonds distant from each other form together a methylenedioxy bridge which is optionally substituted by one or two substituents independently selected from fluorine and methyl,
Rba is 1-4C-alkyl, 1-4C-alkoxy or halogen,
Rbb is 1-4C-alkyl, 1-4C-alkoxy or halogen,
Rca is 1-4C-alkyl, 1-4C-alkoxy or halogen,
Rcb is 1-4C-alkyl, 1-4C-alkoxy or halogen,
Rda is 1-4C-alkyl or halogen,
Rdb is 1-4C-alkyl or halogen,
Rea is 1-4C-alkyl, 1-4C-alkoxy, halogen or hydroxyl,
Reb is 1-4C-alkyl, 1-4C-alkoxy, halogen or hydroxyl,
and the salts, as well as the stereoisomers and salts of the stereoisomers thereof.

2. Compounds according to claim 1, which are from any one of the formulae Ia, Ib and Ic 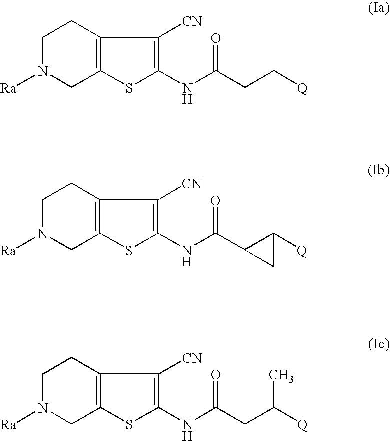
wherein
Ra is —C(O)—N(R11)-R1, in which
either
R1 is methyl, ethyl, propyl, isopropyl or isobutyl,
or
R1 is cyclopropyl, cyclobutyl, cyclopentyl or cyclohexyl,
wherein each of said cyclopropyl, cyclobutyl, cyclopentyl and cyclohexyl may be optionally substituted by one or two substituents independently selected from R12,
or
R1 is phenyl,
wherein said phenyl may be optionally substituted by one or two substituents independently selected from R13,
or
R1 is HarA, in which
either
HarA is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
or
HarA is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
or
HarA is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
or
HarA is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
or
HarA is pyridyl or pyrimidinyl,
wherein each of said HarA may be optionally substituted by one or two substituents independently selected from R13,
or
R1 is HetA, in which
HetA is tetrahydropyranyl, tetrahydrofuryl, tetrahydropyran-2-onyl, tetrahydrofuran-2-onyl, 1N-(acetyl)-piperidinyl, 1N-(acetyl)-pyrrolidinyl, 1N-(formyl)-piperidinyl, 1N-(formyl)-pyrrolidinyl, 1N-(methyl)-piperidin-2-onyl, 1N-(methyl)-pyrrolidin-2-onyl, 1N—(H)-piperidin-2-onyl or 1N—(H)-pyrrolidin-2-onyl,
wherein each of said tetrahydropyranyl and tetrahydrofuranyl may be optionally substituted by one or two substituents independently selected from R16,
or
R1 is (Raa)-methyl, 2-(Raa)-ethyl or 1-(Raa)-ethyl, in which
Raa is cyclopropyl, cyclobutyl, cyclopentyl or cyclohexyl,
wherein each of said cyclopropyl, cyclobutyl, cyclopentyl and cyclohexyl may be optionally substituted by one or two substituents independently selected from R12,
or
R1 is (Raa)-methyl, 2-(Raa)-ethyl or 1-(Raa)-ethyl, in which
Raa is phenyl,
wherein said phenyl may be optionally substituted by one or two substituents independently selected from R13,
or
R1 is (Raa)-methyl, 2-(Raa)-ethyl or 1-(Raa)-ethyl, in which
Raa is HarB, in which
either
HarB is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
or
HarB is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
or
HarB is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
or
HarB is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
or
HarB is pyridyl or pyrimidinyl,
wherein each of said HarB may be optionally substituted by one or two substituents independently selected from R13,
or
R1 is (Raa)-methyl, 2-(Raa)-ethyl or 1-(Raa)-ethyl, in which
Raa is HetC, in which
HetC is tetrahydropyranyl, tetrahydrofuryl, tetrahydropyran-2-onyl, tetrahydrofuran-2-onyl, 1N-(acetyl)-piperidinyl, 1N-(acetyl)-pyrrolidinyl, 1N-(formyl)-piperidinyl, 1N-(formyl)-pyrrolidinyl, 1N-(methyl)-piperidin-2-onyl, 1N-(methyl)-pyrrolidin-2-onyl, 1N—(H)-piperidin-2-onyl, 1N—(H)-pyrrolidin-2-onyl, 3N-(methyl)-oxazolidin-2-onyl, 3N—(H)-oxazolidin-2-onyl, 1N-(methyl)-3N—(H)-imidazolidin-2-onyl, 1N-(methyl)-3N-(methyl)-imidazolidin-2-onyl, or 1N—(H)-3N—(H)-imidazolidin-2-onyl,
wherein each of said tetrahydropyranyl and tetrahydrofuranyl may be optionally substituted by one or two substituents independently selected from R16,
or
R1 is 2-(Raa)-ethyl, in which
Raa is hydroxyl or —OR9, in which
R9 is methyl, ethyl, 2-methoxyethyl or 2-(2-methoxyethoxy)-ethyl,
or
R1 is 2-(Raa)-ethyl, in which
Raa is HarB, in which
HarB is imidazol-1-yl, pyrazol-1-yl, triazol-1-yl, mono- or di-(1-2C-alkyl)-substituted imidazol-1-yl, mono- or di-(1-2C-alkyl)-substituted pyrazol-1-yl, or mono- or di-(1-2C-alkyl)-substituted triazol-1-yl,
wherein each of said HarB may be optionally substituted by one or two substituents independently selected from R13,
or
R1 is 2,3-dihydroxy-propyl,
R11 is hydrogen,
and
either
Q is optionally substituted by Rba and/or Rbb, and is phenyl,
or
Q is optionally substituted by Rca and/or Rcb, and is pyridyl,
or
Q is optionally substituted by Rda and/or Rdb, and is furyl or thienyl,
or
Q is optionally substituted by Rea and/or Reb, and is cyclohexyl or cyclopentyl,
wherein
each R12 may be the same or different and is independently selected from the group consisting of:
methyl, ethyl, fluorine, chlorine, hydroxyl, and methoxy,
each R13 may be the same or different and is independently selected from the group consisting of:
methyl, ethyl, fluorine, chlorine, hydroxyl, methoxy, amino, aminomethyl, mono- or dimethylamino, 2-hydroxy-ethoxy, 2-(1-2C-alkoxy)-ethoxy, hydroxy-1-2C-alkyl, and (1-2C-alkoxy)-1-2C-alkyl,
each R16 may be the same or different and is independently selected from the group consisting of:
methyl, ethyl, fluorine, chlorine, hydroxyl, and methoxy,
Rba is methyl, methoxy, ethoxy, fluorine, chlorine or bromine,
Rbb is methyl, methoxy, ethoxy, fluorine, chlorine or bromine,
Rca is methyl, methoxy, ethoxy, fluorine, chlorine or bromine,
Rcb is methyl, methoxy, ethoxy, fluorine, chlorine or bromine,
Rda is methyl, fluorine, chlorine or bromine,
Rdb is methyl, fluorine, chlorine or bromine,
Rea is methyl, methoxy, ethoxy, fluorine, chlorine or hydroxyl,
Reb is methyl, methoxy, ethoxy, fluorine, chlorine or hydroxyl,
and the salts, as well as the stereoisomers and salts of the stereoisomers thereof.

3. Compounds according to claim 2, which are from any one of the formulae Ia, Ib and Ic,
wherein
Ra is —C(O)—N(R11)-R1, in which
either
R1 is methyl, ethyl, propyl, isopropyl or isobutyl,
or
R1 is cyclopropyl, cyclobutyl, cyclopentyl or cyclohexyl,
wherein each of said cyclopropyl, cyclobutyl, cyclopentyl and cyclohexyl may be optionally substituted by one or two substituents independently selected from R12,
or
R1 is HarA, in which
either
HarA is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
or
HarA is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
or
HarA is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
or
HarA is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
or
HarA is pyridyl,
wherein said pyridyl may be optionally substituted by one or two substituents independently selected from R13,
or
R1 is HetA, in which
HetA is tetrahydropyranyl, tetrahydrofuryl, tetrahydropyran-2-onyl, tetrahydrofuran-2-onyl, 1N-(acetyl)-piperidinyl, 1N-(acetyl)-pyrrolidinyl, 1N-(formyl)-piperidinyl, 1N-(formyl)-pyrrolidinyl, 1N-(methyl)-piperidin-2-onyl, 1N-(methyl)-pyrrolidin-2-onyl, 1N—(H)-piperidin-2-onyl or 1N—(H)-pyrrolidin-2-onyl,
or
R1 is (Raa)-methyl, 2-(Raa)-ethyl or 1-(Raa)-ethyl, in which
Raa is cyclopropyl, cyclobutyl, cyclopentyl or cyclohexyl,
wherein each of said cyclopropyl, cyclobutyl, cyclopentyl and cyclohexyl may be optionally substituted by one or two substituents independently selected from R12,
or
R1 is (Raa)-methyl, 2-(Raa)-ethyl or 1-(Raa)-ethyl, in which
Raa is phenyl,
wherein said phenyl may be optionally substituted by one or two substituents independently selected from R13,
or
R1 is (Raa)-methyl, 2-(Raa)-ethyl or 1-(Raa)-ethyl, in which
Raa is HarB, in which
either
HarB is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
or
HarB is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
or
HarB is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
or
HarB is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
or
HarB is pyridyl,
or
R1 is (Raa)-methyl, 2-(Raa)-ethyl or 1-(Raa)-ethyl, in which
Raa is HetC, in which
HetC is tetrahydropyranyl, tetrahydrofuryl, tetrahydropyran-2-onyl, tetrahydrofuran-2-onyl, 1N-(acetyl)-piperidinyl, 1N-(acetyl)-pyrrolidinyl, 1N-(formyl)-piperidinyl, 1N-(formyl)-pyrrolidinyl, 1N-(methyl)-piperidin-2-onyl, 1N-(methyl)-pyrrolidin-2-onyl, 1N—(H)-piperidin-2-onyl, 1N—(H)-pyrrolidin-2-onyl, 3N-(methyl)-oxazolidin-2-onyl or 3N—(H)-oxazolidin-2-onyl,
or
R1 is 2-(Raa)-ethyl, in which
Raa is hydroxyl or —OR9, in which
R9 is methyl, ethyl or 2-methoxyethyl,
or
R1 is 2-(Raa)-ethyl, in which
Raa is HarB, in which
HarB is imidazol-1-yl, pyrazol-1-yl, triazol-1-yl, mono- or di-(1-2C-alkyl)-substituted imidazol-1-yl, mono- or di-(1-2C-alkyl)-substituted pyrazol-1-yl, or mono- or di-(1-2C-alkyl)-substituted triazol-1-yl,
or
R1 is 2,3-dihydroxy-propyl,
R11 is hydrogen,
and
either
Q is phenyl,
or
Q is 2-methoxyphenyl, 2-ethoxyphenyl, 3-methoxyphenyl, 2-methoxy-5-methyl-phenyl or 2-ethoxy-5-methyl-phenyl,
or
Q is pyridin-2-yl or pyridin-3-yl,
or
Q is furan-2-yl, furan-3-yl, thiophen-2-yl or thiophen-3-yl,
or
Q is cyclohexyl or cyclopentyl,
wherein
each R12 may be the same or different and is independently selected from the group consisting of:
methyl, fluorine, hydroxyl, and methoxy,
each R13 may be the same or different and is independently selected from the group consisting of:
methyl, fluorine, hydroxyl, and methoxy,
and the salts, as well as the stereoisomers and salts of the stereoisomers thereof.

4. Compounds according to claim 2, which are from any one of the formulae Ia, Ib and Ic,
wherein
Ra is —C(O)—N(R11)-R1, in which
either
R1 is methyl or ethyl,
or
R1 is cyclopropyl, cyclobutyl, cyclopentyl or cyclohexyl,
or
R1 is HetA, in which
HetA is tetrahydropyranyl or tetrahydrofuryl,
or
R1 is (Raa)-methyl, 2-(Raa)-ethyl or 1-(Raa)-ethyl, in which
Raa is cyclopropyl, cyclobutyl, cyclopentyl or cyclohexyl,
or
R1 is (Raa)-methyl, 2-(Raa)-ethyl or 1-(Raa)-ethyl, in which
Raa is phenyl,
or
R1 is (Raa)-methyl, 2-(Raa)-ethyl or 1-(Raa)-ethyl, in which
Raa is HarB, in which
either
HarB is 1N-(1-2C-alkyl)-imidazolyl, 1N-(1-2C-alkyl)-pyrazolyl, 1N-(1-2C-alkyl)-triazolyl, 1N-(1-2C-alkyl)-pyrrolyl, 1-2C-alkyl-substituted 1N-(1-2C-alkyl)-imidazolyl, 1-2C-alkyl-substituted 1N-(1-2C-alkyl)-pyrazolyl, 1-2C-alkyl-substituted 1N-(1-2C-alkyl)-triazolyl, or 1-2C-alkyl-substituted 1N-(1-2C-alkyl)-pyrrolyl,
or
HarB is 1N—(H)-imidazolyl, 1N—(H)-pyrazolyl, 1-2C-alkyl-substituted 1N—(H)-imidazolyl, or 1-2C-alkyl-substituted 1N—(H)-pyrazolyl,
or
HarB is 4,5-dihydro-oxazolyl, 4,5-dihydro-thiazolyl, mono- or di-(1-2C-alkyl)-substituted 4,5-dihydro-oxazolyl, or mono- or di-(1-2C-alkyl)-substituted 4,5-dihydro-thiazolyl,
or
HarB is oxazolyl, thiazolyl, isoxazolyl, oxadiazolyl, thiadiazolyl, isothiazolyl, mono- or di-(1-2C-alkyl)-substituted oxazolyl, mono- or di-(1-2C-alkyl)-substituted thiazolyl, mono- or di-(1-2C-alkyl)-substituted isoxazolyl, mono- or di-(1-2C-alkyl)-substituted oxadiazolyl, mono- or di-(1-2C-alkyl)-substituted thiadiazolyl, or mono- or di-(1-2C-alkyl)-substituted isothiazolyl,
or
HarB is pyridyl,
or
R1 is (Raa)-methyl, 2-(Raa)-ethyl or 1-(Raa)-ethyl, in which
Raa is HetC, in which
HetC is tetrahydropyranyl, tetrahydrofuryl, tetrahydropyran-2-onyl, tetrahydrofuran-2-onyl, 1N-(acetyl)-piperidinyl, 1N-(acetyl)-pyrrolidinyl, 1N-(formyl)-piperidinyl, 1N-(formyl)-pyrrolidinyl, 1N-(methyl)-piperidin-2-onyl, 1N-(methyl)-pyrrolidin-2-onyl, 1N—(H)-piperidin-2-onyl, 1N—(H)-pyrrolidin-2-onyl, 3N-(methyl)-oxazolidin-2-onyl or 3N—(H)-oxazolidin-2-onyl,
or
R1 is 2-(Raa)-ethyl, in which
Raa is hydroxyl or —OR9, in which
R9 is methyl, ethyl or 2-methoxyethyl,
or
R1 is 2-(Raa)-ethyl, in which
Raa is HarB, in which
HarB is imidazol-1-yl, pyrazol-1-yl, triazol-1-yl, mono- or di-(1-2C-alkyl)-substituted imidazol-1-yl, mono- or di-(1-2C-alkyl)-substituted pyrazol-1-yl, or mono- or di-(1-2C-alkyl)-substituted triazol-1-yl,
or
R1 is 2,3-dihydroxy-propyl,
R11 is hydrogen,
and
either
Q is phenyl,
or
Q is 2-methoxyphenyl, 2-ethoxyphenyl or 3-methoxyphenyl,
or
Q is pyridin-2-yl or pyridin-3-yl,
or
Q is furan-2-yl,
or
Q is cyclohexyl,
and the salts, as well as the stereoisomers and salts of the stereoisomers thereof.

5. Compounds according to claim 1, in which Ra is selected from the following meanings 1) to 72):

| | Ra |
|---|---|
| 1) | 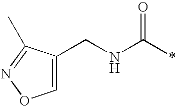 |
| 2) | 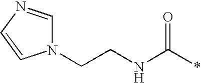 |
| 3) | 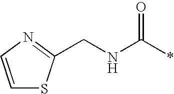 |
| 4) | 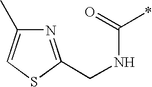 |
| 5) | 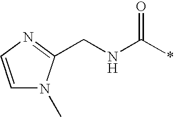 |
| 6) | 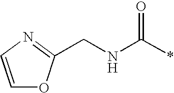 |
| 7) | 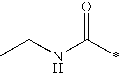 |
| 8) | 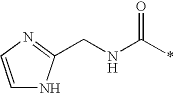 |
| 9) | 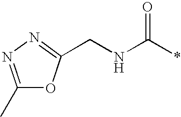 |
| 10) | 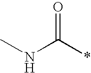 |
| 11) | 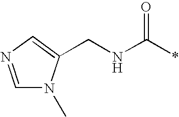 |
| 12) | 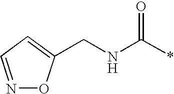 |
| 13) | 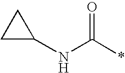 |
| 14) | 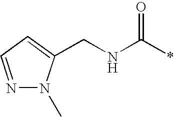 |
| 15) | 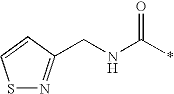 |
| 16) | 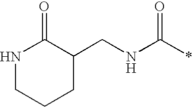 |
| 17) | 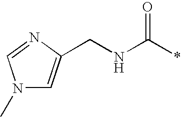 |
| 18) | 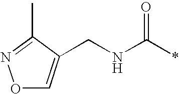 |
| 19) | 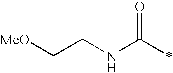 |
| 20) | 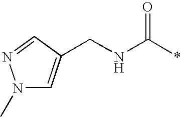 |
| 21) | 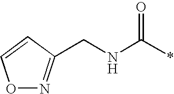 |
| 22) | 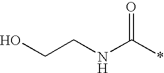 |
| 23) | 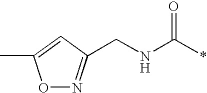 |
| 24) | 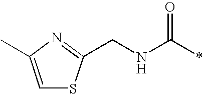 |
| 25) | 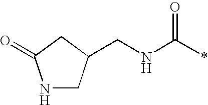 |
| 26) | 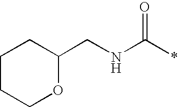 |
| 27) | 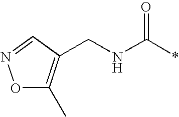 |
| 28) | 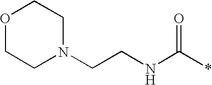 |
| 29) | 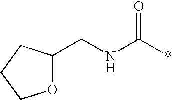 |
| 30) | 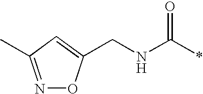 |
| 31) | 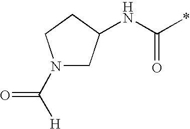 |
| 32) | 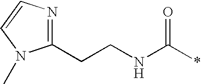 |
| 33) | 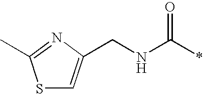 |
| 34) | 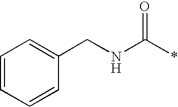 |
| 35) | 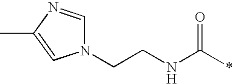 |
| 36) | 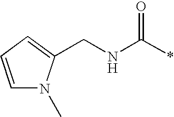 |
| 37) | 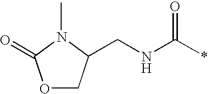 |
| 38) | 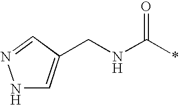 |
| 39) | 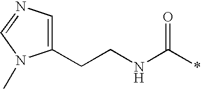 |
| 40) | 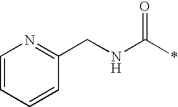 |
| 41) | 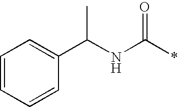 |
| 42) | 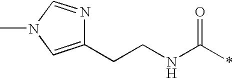 |
| 43) | 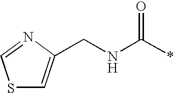 |
| 44) | 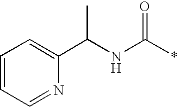 |
| 45) | 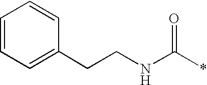 |
| 46) | 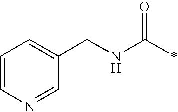 |
| 47) | 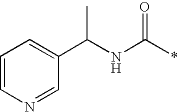 |
| 48) | 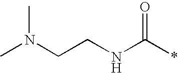 |
| 49) | 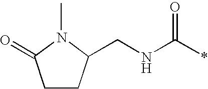 |
| 50) | 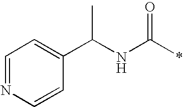 |
| 51) | 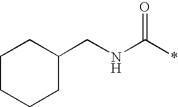 |
| 52) | 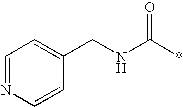 |
| 53) | 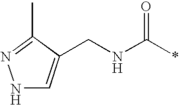 |
| 54) | 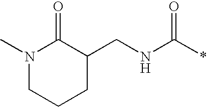 |
| 55) | 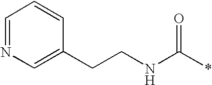 |
| 56) | 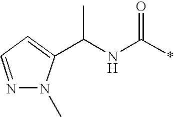 |
| 57) | 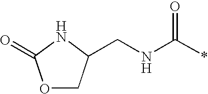 |
| 58) | 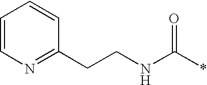 |
| 59) | 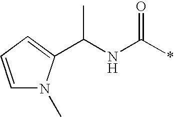 |
| 60) | 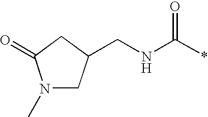 |
| 61) | 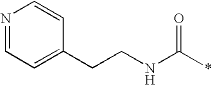 |
| 62) | 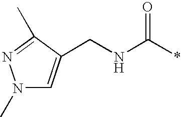 |
| 63) | 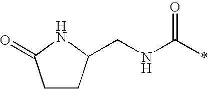 |
| 64) | 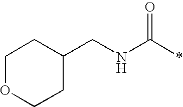 |
| 65) | 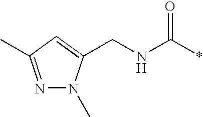 |
| 66) | 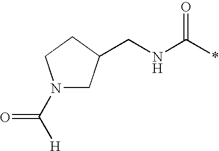 |
| 67) | 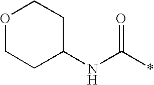 |
| 68) | 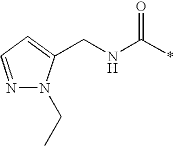 |
| 69) | 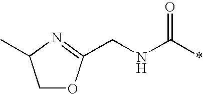 |
| 70) | 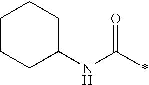 |
| 71) | 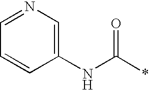 |
| 72) | 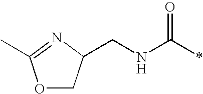 |

and the salts, as well as the stereoisomers and salts of the stereoisomers thereof.

6. A compound of formula I according to claim 1, which is selected from
1. 3-Cyano-2-[3-(2-methoxy-phenyl)-butanoylamino]-4,7-dihydro-5H-thieno[2,3-c]pyridine-6-carboxylic acid(pyridin-2-ylmethyl)-amide
2. 3-Cyano-2-[3-(2-ethoxy-phenyl)-butanoylamino]-4,7-dihydro-5H-thieno[2,3-c]pyridine-6-carboxylic acid(pyridin-2-ylmethyl)-amide
3. 3-Cyano-2-[3-(3-methoxy-phenyl)-propanoylamino]-4,7-dihydro-5H-thieno[2,3-c]pyridine-6-carboxylic acid(pyridin-2-ylmethyl)-amide
4. 3-Cyano-2-[3-(2-methoxy-phenyl)-propanoylamino]-4,7-dihydro-5H-thieno[2,3-c]pyridine-6-carboxylic acid(pyridin-2-ylmethyl)-amide
5. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(pyridin-2-ylmethyl)-amide
6. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(pyridin-2-ylmethyl)-amide
7. 3-Cyano-2-((RS)-3-phenyl-butanoylamino)-4,7-dihydro-5H-thieno[2,3-c]pyridine-6-carboxylic acid(pyridin-2-ylmethyl)-amide
8. 3-Cyano-2-(3-phenyl-propanoylamino)-4,7-dihydro-5H-thieno[2,3-c]pyridine-6-carboxylic acid(pyridin-2-ylmethyl)-amide
9. 3-Cyano-2-(3-cyclohexyl-propanoylamino)-4,7-dihydro-5H-thieno[2,3-c]pyridine-6-carboxylic acid(pyridin-2-ylmethyl)-amide
10. 3-Cyano-2-[(RS)-3-(2-methoxy-phenyl)-butanoylamino]-4,7-dihydro-5H-thieno[2,3-c]pyridine-6-carboxylic acid(pyridin-4-ylmethyl)-amide
11. 3-Cyano-2-[(RS)-3-(2-ethoxy-phenyl)-butanoylamino]-4,7-dihydro-5H-thieno[2,3-c]pyridine-6-carboxylic acid(pyridin-4-ylmethyl)-amide
12. 3-Cyano-2-[3-(3-methoxy-phenyl)-propanoylamino]-4,7-dihydro-5H-thieno[2,3-c]pyridine-6-carboxylic acid(pyridin-4-ylmethyl)-amide
13. 3-Cyano-2-[3-(2-methoxy-phenyl)-propanoylamino]-4,7-dihydro-5H-thieno[2,3-c]pyridine-6-carboxylic acid(pyridin-4-ylmethyl)-amide
14. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(pyridin-4-ylmethyl)-amide
15. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(pyridin-4-ylmethyl)-amide
16. 3-Cyano-2-((RS)-3-phenyl-butanoylamino)-4,7-dihydro-5H-thieno[2,3-c]pyridine-6-carboxylic acid(pyridin-4-ylmethyl)-amide
17. 3-Cyano-2-(3-phenyl-propanoylamino)-4,7-dihydro-5H-thieno[2,3-c]pyridine-6-carboxylic acid(pyridin-4-ylmethyl)-amide
18. 3-Cyano-2-(3-cyclohexyl-propanoylamino)-4,7-dihydro-5H-thieno[2,3-c]pyridine-6-carboxylic acid(pyridin-4-ylmethyl)-amide
19. 3-Cyano-2-[(RS)-3-(2-methoxy-phenyl)-butanoylamino]-4,7-dihydro-5H-thieno[2,3-c]pyridine-6-carboxylic acid(2-pyridin-2-yl-ethyl)-amide
20. 3-Cyano-2-[(RS)-3-(2-ethoxy-phenyl)-butanoylamino]-4,7-dihydro-5H-thieno[2,3-c]pyridine-6-carboxylic acid(2-pyridin-2-yl-ethyl)-amide
21. 3-Cyano-2-[3-(3-methoxy-phenyl)-propanoylamino]-4,7-dihydro-5H-thieno[2,3-c]pyridine-6-carboxylic acid(2-pyridin-2-yl-ethyl)-amide
22. 3-Cyano-2-[3-(2-methoxy-phenyl)-propanoylamino]-4,7-dihydro-5H-thieno[2,3-c]pyridine-6-carboxylic acid(2-pyridin-2-yl-ethyl)-amide
23. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(2-pyridin-2-yl-ethyl)-amide
24. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(2-pyridin-2-yl-ethyl)-amide
25. 3-Cyano-2-((RS)-3-phenyl-butanoylamino)-4,7-dihydro-5H-thieno[2,3-c]pyridine-6-carboxylic acid(2-pyridin-2-yl-ethyl)-amide
26. 3-Cyano-2-(3-phenyl-propanoylamino)-4,7-dihydro-5H-thieno[2,3-c]pyridine-6-carboxylic acid(2-pyridin-2-yl-ethyl)-amide
27. 3-Cyano-2-(3-cyclohexyl-propanoylamino)-4,7-dihydro-5H-thieno[2,3-c]pyridine-6-carboxylic acid(2-pyridin-2-yl-ethyl)-amide
28. 3-Cyano-2-[(RS)-3-(2-methoxy-phenyl)-butanoylamino]-4,7-dihydro-5H-thieno[2,3-c]pyridine-6-carboxylic acid(2-pyridin-3-yl-ethyl)-amide
29. 3-Cyano-2-[(RS)-3-(2-ethoxy-phenyl)-butanoylamino]-4,7-dihydro-5H-thieno[2,3-c]pyridine-6-carboxylic acid(2-pyridin-3-yl-ethyl)-amide
30. 3-Cyano-2-[(RS)-3-(3-methoxy-phenyl)-propanoylamino]-4,7-dihydro-5H-thieno[2,3-c]pyridine-6-carboxylic acid(2-pyridin-3-yl-ethyl)-amide
31. 3-Cyano-2-[3-(2-methoxy-phenyl)-propanoylamino]-4,7-dihydro-5H-thieno[2,3-c]pyridine-6-carboxylic acid(2-pyridin-3-yl-ethyl)-amide
32. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(2-pyridin-3-yl-ethyl)-amide
33. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(2-pyridin-3-yl-ethyl)-amide
34. 3-Cyano-2-((RS)-3-phenyl-butanoylamino)-4,7-dihydro-5H-thieno[2,3-c]pyridine-6-carboxylic acid(2-pyridin-3-yl-ethyl)-amide
35. 3-Cyano-2-(3-phenyl-propanoylamino)-4,7-dihydro-5H-thieno[2,3-c]pyridine-6-carboxylic acid(2-pyridin-3-yl-ethyl)-amide
36. 3-Cyano-2-(3-cyclohexyl-propanoylamino)-4,7-dihydro-5H-thieno[2,3-c]pyridine-6-carboxylic acid(2-pyridin-3-yl-ethyl)-amide
37. 3-Cyano-2-[(RS)-3-(2-methoxy-phenyl)-butanoylamino]-4,7-dihydro-5H-thieno[2,3-c]pyridine-6-carboxylic acid(2-pyridin-4-yl-ethyl)-amide
38. 3-Cyano-2-[(RS)-3-(2-ethoxy-phenyl)-butanoylamino]-4,7-dihydro-5H-thieno[2,3-c]pyridine-6-carboxylic acid(2-pyridin-4-yl-ethyl)-amide
39. 3-Cyano-2-[3-(3-methoxy-phenyl)-propanoylamino]-4,7-dihydro-5H-thieno[2,3-c]pyridine-6-carboxylic acid(2-pyridin-4-yl-ethyl)-amide
40. 3-Cyano-2-[3-(2-methoxy-phenyl)-propanoylamino]-4,7-dihydro-5H-thieno[2,3-c]pyridine-6-carboxylic acid(2-pyridin-4-yl-ethyl)-amide
41. 3-Cyano-2-({1-[(1RS,2RS)-2-(3-methoxy-phenyl)-cyclopropyl]-methanoyl}-amino)-4,7-dihydro-5H-thieno[2,3-c]pyridine-6-carboxylic acid(2-pyridin-4-yl-ethyl)-amide
42. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(2-pyridin-4-yl-ethyl)-amide
43. 3-Cyano-2-(3-phenyl-butanoylamino)-4,7-dihydro-5H-thieno[2,3-c]pyridine-6-carboxylic acid(2-pyridin-4-yl-ethyl)-amide
44. 3-Cyano-2-(3-phenyl-propanoylamino)-4,7-dihydro-5H-thieno[2,3-c]pyridine-6-carboxylic acid(2-pyridin-4-yl-ethyl)-amide
45. 3-Cyano-2-(3-cyclohexyl-propanoylamino)-4,7-dihydro-5H-thieno[2,3-c]pyridine-6-carboxylic acid(2-pyridin-4-yl-ethyl)-amide
46. 3-Cyano-2-[(RS)-3-(2-methoxy-phenyl)-butanoylamino]-4,7-dihydro-5H-thieno[2,3-c]pyridine-6-carboxylic acid(pyridin-3-ylmethyl)-amide
47. 3-Cyano-2-[(RS)-3-(2-ethoxy-phenyl)-butanoylamino]-4,7-dihydro-5H-thieno[2,3-c]pyridine-6-carboxylic acid(pyridin-3-ylmethyl)-amide
48. 3-Cyano-2-[3-(3-methoxy-phenyl)-propanoylamino]-4,7-dihydro-5H-thieno[2,3-c]pyridine-6-carboxylic acid(pyridin-3-ylmethyl)-amide
49. 3-Cyano-2-[3-(2-methoxy-phenyl)-propanoylamino]-4,7-dihydro-5H-thieno[2,3-c]pyridine-6-carboxylic acid(pyridin-3-ylmethyl)-amide
50. 3-Cyano-2-({1-[1RS,2RS)-2-(3-methoxy-phenyl)-cyclopropyl]-methanoyl}-amino)-4,7-dihydro-5H-thieno[2,3-c]pyridine-6-carboxylic acid(pyridin-3-ylmethyl)-amide
51. 3-Cyano-2-({1-[(1RS,2RS)-2-(2-methoxy-phenyl)-cyclopropyl]-methanoyl}-amino)-4,7-dihydro-5H-thieno[2,3-c]pyridine-6-carboxylic acid(pyridin-3-ylmethyl)-amide
52. 3-Cyano-2-((RS)-3-phenyl-butanoylamino)-4,7-dihydro-5H-thieno[2,3-c]pyridine-6-carboxylic acid(pyridin-3-ylmethyl)-amide
53. 3-Cyano-2-(3-phenyl-propanoylamino)-4,7-dihydro-5H-thieno[2,3-c]pyridine-6-carboxylic acid(pyridin-3-ylmethyl)-amide
54. 3-Cyano-2-(3-cyclohexyl-propanoylamino)-4,7-dihydro-5H-thieno[2,3-c]pyridine-6-carboxylic acid(pyridin-3-ylmethyl)-amide
55. 3-Cyano-2-[3-(3-methoxy-phenyl)-propanoylamino]-4,7-dihydro-5H-thieno[2,3-c]pyridine-6-carboxylic acid phenethyl-amide
56. 3-Cyano-2-[3-(2-ethoxy-phenyl)-propanoylamino]-4,7-dihydro-5H-thieno[2,3-c]pyridine-6-carboxylic acid phenethyl-amide
57. 3-Cyano-2-[3-(2-methoxy-5-methyl-phenyl)-propanoylamino]-4,7-dihydro-5H-thieno[2,3-c]pyridine-6-carboxylic acid(2-pyridin-2-yl-ethyl)-amide
58. 3-Cyano-2-[3-(2-methoxy-phenyl)-propanoylamino]-4,7-dihydro-5H-thieno[2,3-c]pyridine-6-carboxylic acid(2-morpholin-4-yl-ethyl)-amide
59. 3-Cyano-2-[3-(3-methoxy-phenyl)-propanoylamino]-4,7-dihydro-5H-thieno[2,3-c]pyridine-6-carboxylic acid(2-morpholin-4-yl-ethyl)-amide
60. 3-Cyano-2-[3-(2-methoxy-phenyl)-propanoylamino]-4,7-dihydro-5H-thieno[2,3-c]pyridine-6-carboxylic acid(2-methoxy-ethyl)-amide
61. 3-Cyano-2-[3-(3-methoxy-phenyl)-propanoylamino]-4,7-dihydro-5H-thieno[2,3-c]pyridine-6-carboxylic acid(2-methoxy-ethyl)-amide
62. 3-Cyano-2-[3-(2-methoxy-phenyl)-propanoylamino]-4,7-dihydro-5H-thieno[2,3-c]pyridine-6-carboxylic acid(2-imidazol-1-yl-ethyl)-amide
63. 3-Cyano-2-[3-(3-methoxy-phenyl)-propanoylamino]-4,7-dihydro-5H-thieno[2,3-c]pyridine-6-carboxylic acid(2-imidazol-1-yl-ethyl)-amide
64. 3-Cyano-2-[3-(2-methoxy-phenyl)-propanoylamino]-4,7-dihydro-5H-thieno[2,3-c]pyridine-6-carboxylic acid(3-methyl-3H-imidazol-4-ylmethyl)-amide
65. 3-Cyano-2-[3-(3-methoxy-phenyl)-propanoylamino]-4,7-dihydro-5H-thieno[2,3-c]pyridine-6-carboxylic acid(3-methyl-3H-imidazol-4-ylmethyl)-amide
66. 3-Cyano-2-[3-(2-methoxy-phenyl)-propanoylamino]-4,7-dihydro-5H-thieno[2,3-c]pyridine-6-carboxylic acid(1-methyl-1H-imidazol-4-ylmethyl)-amide
67. 3-Cyano-2-[3-(3-methoxy-phenyl)-propanoylamino]-4,7-dihydro-5H-thieno[2,3-c]pyridine-6-carboxylic acid(1-methyl-1H-imidazol-4-ylmethyl)-amide
68. 3-Cyano-2-[3-(2-methoxy-phenyl)-propanoylamino]-4,7-dihydro-5H-thieno[2,3-c]pyridine-6-carboxylic acid [2-(3-methyl-3H-imidazol-4-yl)-ethyl]amide
69. 3-Cyano-2-[3-(3-methoxy-phenyl)-propanoylamino]-4,7-dihydro-5H-thieno[2,3-c]pyridine-6-carboxylic acid [2-(3-methyl-3H-imidazol-4-yl)-ethyl]amide
70. 3-Cyano-2-[3-(2-methoxy-phenyl)-propanoylamino]-4,7-dihydro-5H-thieno[2,3-c]pyridine-6-carboxylic acid [2-(1-methyl-1H-imidazol-4-yl)-ethyl]-amide
71. 3-Cyano-2-[3-(3-methoxy-phenyl)-propanoylamino]-4,7-dihydro-5H-thieno[2,3-c]pyridine-6-carboxylic acid [2-(1-methyl-1H-imidazol-4-yl)-ethyl]-amide
72. 3-Cyano-2-[3-(2-methoxy-phenyl)-propanoylamino]-4,7-dihydro-5H-thieno[2,3-c]pyridine-6-carboxylic acid(5-methyl-[1,3,4]oxadiazol-2-ylmethyl)-amide
73. 3-Cyano-2-[3-(3-methoxy-phenyl)-propanoylamino]-4,7-dihydro-5H-thieno[2,3-c]pyridine-6-carboxylic acid(5-methyl-[1,3,4]oxadiazol-2-ylmethyl)-amide
74. 3-Cyano-2-[3-(2-methoxy-phenyl)-propanoylamino]-4,7-dihydro-5H-thieno[2,3-c]pyridine-6-carboxylic acid(3-morpholin-4-yl-propyl)-amide
75. 3-Cyano-2-[3-(3-methoxy-phenyl)-propanoylamino]-4,7-dihydro-5H-thieno[2,3-c]pyridine-6-carboxylic acid(3-morpholin-4-yl-propyl)-amide
76. 3-Cyano-2-[3-(2-methoxy-phenyl)-propanoylamino]-4,7-dihydro-5H-thieno[2,3-c]pyridine-6-carboxylic acid(1-methyl-1H-pyrrol-2-ylmethyl)-amide
77. 3-Cyano-2-[3-(3-methoxy-phenyl)-propanoylamino]-4,7-dihydro-5H-thieno[2,3-c]pyridine-6-carboxylic acid(1-methyl-1H-pyrrol-2-ylmethyl)-amide
78. 3-Cyano-2-[3-(2-methoxy-phenyl)-propanoylamino]-4,7-dihydro-5H-thieno[2,3-c]pyridine-6-carboxylic acid(5-methyl-isoxazol-3-ylmethyl)-amide
79. 3-Cyano-2-[3-(3-methoxy-phenyl)-propanoylamino]-4,7-dihydro-5H-thieno[2,3-c]pyridine-6-carboxylic acid(5-methyl-isoxazol-3-ylmethyl)-amide
80. 3-Cyano-2-[3-(2-methoxy-phenyl)-propanoylamino]-4,7-dihydro-5H-thieno[2,3-c]pyridine-6-carboxylic acid(tetrahydro-furan-2-ylmethyl)-amide
81. 3-Cyano-2-[3-(3-methoxy-phenyl)-propanoylamino]-4,7-dihydro-5H-thieno[2,3-c]pyridine-6-carboxylic acid(tetrahydro-furan-2-ylmethyl)-amide
82. 3-Cyano-2-[3-(2-methoxy-phenyl)-propanoylamino]-4,7-dihydro-5H-thieno[2,3-c]pyridine-6-carboxylic acid carbamoylmethyl-amide
83. 3-Cyano-2-[3-(3-methoxy-phenyl)-propanoylamino]-4,7-dihydro-5H-thieno[2,3-c]pyridine-6-carboxylic acid carbamoylmethyl-amide
84. 3-Cyano-2-[3-(2-methoxy-phenyl)-propanoylamino]-4,7-dihydro-5H-thieno[2,3-c]pyridine-6-carboxylic acid(2,5-dimethyl-2H-pyrazol-3-ylmethyl)-amide
85. 3-Cyano-2-[3-(3-methoxy-phenyl)-propanoylamino]-4,7-dihydro-5H-thieno[2,3-c]pyridine-6-carboxylic acid(2,5-dimethyl-2H-pyrazol-3-ylmethyl)-amide
86. 3-Cyano-2-[3-(2-methoxy-phenyl)-propanoylamino]-4,7-dihydro-5H-thieno[2,3-c]pyridine-6-carboxylic acid(1-methyl-1H-pyrrol-2-ylmethyl)-amide
87. 3-Cyano-2-[3-(3-methoxy-phenyl)-propanoylamino]-4,7-dihydro-5H-thieno[2,3-c]pyridine-6-carboxylic acid(1-methyl-1H-pyrrol-2-ylmethyl)-amide
88. 3-Cyano-2-[3-(2-methoxy-phenyl)-propanoylamino]-4,7-dihydro-5H-thieno[2,3-c]pyridine-6-carboxylic acid(1,3-dimethyl-1H-pyrazol-4-ylmethyl)-amide
89. 3-Cyano-2-[3-(3-methoxy-phenyl)-propanoylamino]-4,7-dihydro-5H-thieno[2,3-c]pyridine-6-carboxylic acid(1,3-dimethyl-1H-pyrazol-4-ylmethyl)-amide
90. 3-Cyano-2-[3-(2-methoxy-phenyl)-propanoylamino]-4,7-dihydro-5H-thieno[2,3-c]pyridine-6-carboxylic acid(isoxazol-3-ylmethyl)-amide
91. 3-Cyano-2-[3-(3-methoxy-phenyl)-propanoylamino]-4,7-dihydro-5H-thieno[2,3-c]pyridine-6-carboxylic acid(isoxazol-3-ylmethyl)-amide
92. 3-Cyano-2-[3-(2-methoxy-phenyl)-propanoylamino]-4,7-dihydro-5H-thieno[2,3-c]pyridine-6-carboxylic acid(3-methyl-isoxazol-5-ylmethyl)-amide
93. 3-Cyano-2-[3-(3-methoxy-phenyl)-propanoylamino]-4,7-dihydro-5H-thieno[2,3-c]pyridine-6-carboxylic acid(3-methyl-isoxazol-5-ylmethyl)-amide
94. 3-Cyano-2-[3-(2-methoxy-phenyl)-propanoylamino]-4,7-dihydro-5H-thieno[2,3-c]pyridine-6-carboxylic acid(oxazol-2-ylmethyl)-amide
95. 3-Cyano-2-[3-(3-methoxy-phenyl)-propanoylamino]-4,7-dihydro-5H-thieno[2,3-c]pyridine-6-carboxylic acid(oxazol-2-ylmethyl)-amide
96. 3-Cyano-2-[3-(2-methoxy-phenyl)-propanoylamino]-4,7-dihydro-5H-thieno[2,3-c]pyridine-6-carboxylic acid(1-methyl-1H-pyrazol-4-ylmethyl)-amide
97. 3-Cyano-2-[3-(3-methoxy-phenyl)-propanoylamino]-4,7-dihydro-5H-thieno[2,3-c]pyridine-6-carboxylic acid(1-methyl-1H-pyrazol-4-ylmethyl)-amide
98. 3-Cyano-2-[3-(3-methoxy-phenyl)-propanoylamino]-4,7-dihydro-5H-thieno[2,3-c]pyridine-6-carboxylic acid(2-methyl-2H-pyrazol-3-ylmethyl)-amide
99. 3-Cyano-2-[3-(2-methoxy-phenyl)-propanoylamino]-4,7-dihydro-5H-thieno[2,3-c]pyridine-6-carboxylic acid(2-methyl-2H-pyrazol-3-ylmethyl)-amide
100. 3-Cyano-2-[3-(2-methoxy-phenyl)-propanoylamino]-4,7-dihydro-5H-thieno[2,3-c]pyridine-6-carboxylic acid(1-methyl-1H-imidazol-2-ylmethyl)-amide
101. 3-Cyano-2-[3-(3-methoxy-phenyl)-propanoylamino]-4,7-dihydro-5H-thieno[2,3-c]pyridine-6-carboxylic acid(1-methyl-1H-imidazol-2-ylmethyl)-amide
102. 3-Cyano-2-[3-(3-methoxy-phenyl)-propanoylamino]-4,7-dihydro-5H-thieno[2,3-c]pyridine-6-carboxylic acid(3-methyl-1H-pyrazol-4-ylmethyl)-amide
103. 3-Cyano-2-[3-(2-methoxy-phenyl)-propanoylamino]-4,7-dihydro-5H-thieno[2,3-c]pyridine-6-carboxylic acid(3-methyl-1H-pyrazol-4-ylmethyl)-amide
104. 3-Cyano-2-[3-(3-methoxy-phenyl)-propanoylamino]-4,7-dihydro-5H-thieno[2,3-c]pyridine-6-carboxylic acid [2-(1-methyl-1H-imidazol-4-yl)-ethyl]-amide
105. 3-Cyano-2-[3-(2-methoxy-phenyl)-propanoylamino]-4,7-dihydro-5H-thieno[2,3-c]pyridine-6-carboxylic acid [2-(1-methyl-1H-imidazol-4-yl)-ethyl]-amide
106. 3-Cyano-2-[3-(2-ethoxy-phenyl)-propanoylamino]-4,7-dihydro-5H-thieno[2,3-c]pyridine-6-carboxylic acid ethylamide
107. 3-Cyano-2-[3-(2-methoxy-5-methyl-phenyl)-propanoylamino]-4,7-dihydro-5H-thieno[2,3-c]pyridine-6-carboxylic acid ethylamide
108. 3-Cyano-2-[3-(3-methoxy-phenyl)-propanoylamino]-4,7-dihydro-5H-thieno[2,3-c]pyridine-6-carboxylic acid ethylamide
109. 3-Cyano-2-(3-furan-2-yl-propanoylamino)-4,7-dihydro-5H-thieno[2,3-c]pyridine-6-carboxylic acid ethylamide
110. 3-Cyano-2-[3-(3-methoxy-phenyl)-butanoylamino]-4,7-dihydro-5H-thieno[2,3-c]pyridine-6-carboxylic acid ethylamide
111. 3-Cyano-2-[3-(2-methoxy-phenyl)-butanoylamino]-4,7-dihydro-5H-thieno[2,3-c]pyridine-6-carboxylic acid ethylamide
112. 3-Cyano-2-[3-(2-ethoxy-phenyl)-butanoyl amino]-4,7-dihydro-5H-thieno[2,3-c]pyridine-6-carboxylic acid ethylamide
113. 3-Cyano-2-[3-(2-methoxy-phenyl)-propanoylamino]-4,7-dihydro-5H-thieno[2,3-c]pyridine-6-carboxylic acid ethylamide
114. 3-Cyano-2-(3-phenyl-butanoyl amino)-4,7-dihydro-5H-thieno[2,3-c]pyridine-6-carboxylic acid ethylamide
115. 3-Cyano-2-(3-phenyl-propanoylamino)-4,7-dihydro-5H-thieno[2,3-c]pyridine-6-carboxylic acid ethylamide
116. 3-Cyano-2-({1-[(RS)-2-(2-methoxy-phenyl)-cyclopropyl]-methanoyl}-amino)-4,7-dihydro-5H-thieno[2,3-c]pyridine-6-carboxylic acid ethylamide
117. 3-Cyano-2-({1-[(RS)-2-(3-methoxy-phenyl)-cyclopropyl]-methanoyl}-amino)-4,7-dihydro-5H-thieno[2,3-c]pyridine-6-carboxylic acid ethylamide, and
118. 3-Cyano-2-(3-cyclohexyl-propanoylamino)-4,7-dihydro-5H-thieno[2,3-c]pyridine-6-carboxylic acid ethylamide
or a salt, stereoisomer or salt of a stereoisomer thereof.

7. Compounds according to claim 1 for use in the treatment of diseases.

8. A compound according claim 1 or a pharmaceutically acceptable salt thereof, or a pharmaceutical composition comprising a compound according to claim 1 or a pharmaceutically acceptable salt thereof, for the treatment of (hyper) proliferative diseases and/or disorders responsive to the induction of apoptosis.

9. A pharmaceutical composition comprising one or more compounds according to claim 1 together with customary pharmaceutical auxiliaries and/or excipients.

9. (canceled)

10. A method for treating, preventing or ameliorating (hyper)proliferative diseases and/or disorders responsive to induction of apoptosis, such as, for example, benign or malignant neoplasia, e.g. cancer, in a mammal comprising administering a therapeutically effective amount of one or more compounds according to claim 1 to said mammal in need thereof.

11. A combination comprising
a first active ingredient, which is at least one compound according to claim 1, and a second active ingredient, which is at least one anti-cancer agent selected from the group consisting of chemotherapeutic anti-cancer agents and target-specific anti-cancer agents, for separate, sequential, simultaneous, concurrent or chronologically staggered use in therapy, such as e.g. in therapy of benign or malignant neoplasia, e.g. cancer.

12. A method for treating, preventing or ameliorating hyperproliferative diseases and/or disorders responsive to induction of apoptosis, such as, for example, benign or malignant neoplasia, e.g. cancer, in a patient comprising administering separately, simultaneously, concurrently, sequentially or chronologically staggered to said patient in need thereof an amount of a first active compound, which is a compound according to claim 1, and an amount of at least one second active compound, said second active compound being an anti-cancer agent selected from the group consisting of chemotherapeutic anti-cancer agents and target-specific anti-cancer agents, wherein the amounts of the first active compound and said second active compound result in a therapeutic effect.

13. The combination or method according to claim 11, in which said chemotherapeutic anti-cancer agents are selected from (i) alkylating/carbamylating agents including Cyclophosphamid, Ifosfamid, Thiotepa, Melphalan and chloroethylnitrosourea; (ii) platinum derivatives including cis-platin, oxaliplatin, satraplatin and carboplatin; (iii) antimitotic agents/tubulin inhibitors including vinca alkaloids, such as e.g. vincristine, vinblastine or vinorelbine, taxanes, such as e.g. Paclitaxel, Docetaxel and analogs as well as formulations and conjugates thereof, and epothilones, such as e.g. Epothilone B, Azaepothilone or ZK-EPO; (iv) topoisomerase inhibitors including anthracyclines, such as e.g. Doxorubicin, epipodophyllotoxines, such as e.g. Etoposide, and camptothecin and camptothecin analogs, such as e.g. Irinotecan or Topotecan; (v) pyrimidine antagonists including 5-fluorouracil, Capecitabine, Arabinosylcytosine/Cytarabin and Gemcitabine; (vi) purin antagonists including 6-mercaptopurine, 6-thioguanine and fludarabine; and (vii) folic acid antagonists including methotrexate and pemetrexed.

14. The combination or method according to claim 11, in which said target-specific anti-cancer agents are selected from (i) kinase inhibitors including Imatinib, ZD-1839/Gefitinib, BAY43-9006/Sorafenib, SU11248/Sunitinib and OSI-774/Erlotinib, Dasatinib, Lapatinib, Vatalanib, Vandetanib and Pazopanib; (ii) proteasome inhibitors including PS-341/Bortezomib; (iii) histone deacetylase inhibitors including SAHA, PXD101, MS275, MGCD0103, Depsipeptide/FK228, NVP-LBH589, NVP-LAQ824, Valproic acid (VPA) and butyrates; (iv) heat shock protein 90 inhibitors including 17-allylaminogeldanamycin (17-AAG); (v) vascular targeting agents (VAT) including combretastatin A4 phosphate and AVE8062/AC7700, and anti-angiogenic drugs including VEGF antibodies, such as e.g. Bevacizumab, and KDR tyrosine kinase inhibitors, such as e.g. PTK787/ZK222584 (Vatalanib), Vandetanib or Pazopanib; (vi) monoclonal antibodies including Trastuzumab, Rituximab, Alemtuzumab, Tositumomab, Cetuximab, Bevacizumab and Panitumumab as well as mutants and conjugates of monoclonal antibodies, such as e.g. Gemtuzumab ozogamicin or Ibritumomab tiuxetan, and antibody fragments; (vii) oligonucleotide based therapeutics including G-3139/Oblimersen; (viii) Toll-like receptor/TLR 9 agonists including Promune®, TLR 7 agonists including Imiquimod and Isatoribine and analogues thereof, or TLR 7/8 agonists including Resiquimod as well as immunostimulatory RNA as TLR 7/8 agonists; (ix) protease inhibitors; (x) hormonal therapeutics including anti-estrogens, such as e.g. Tamoxifen or Raloxifen, anti-androgens, such as e.g. Flutamide or Casodex, LHRH analogs, such as e.g. Luprolide, Goserelin or Triptorelin, and aromatase inhibitors;
bleomycin; retinoids including all-trans retinoic acid (ATRA); DNA methyltransferase inhibitors including the 2-deoxycytidine derivative Decitabine and 5-Azacytidine; alanosine; cytokines including interleukin-2; interferons including interferon α2 and interferon-γ; and death receptor agonists including TRAIL, DR4/5 agonistic antibodies, FasL and TNF-R agonists, such as e.g. TRAIL receptor agonists like mapatumumab or lexatumumab.

15. The method according to claim 10, in which said cancer is selected from the group consisting of cancer of the breast, bladder, bone, brain, central and peripheral nervous system, colon, endocrine glands, esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva; inherited cancers, retinomblastoma and Wilms tumor;
leukemia, lymphoma, non-Hodgkins disease, chronic and acute myeloid leukaemia, acute lymphoblastic leukemia, Hodgkins disease, multiple myeloma and T-cell lymphoma; myelodysplastic syndrome, plasma cell neoplasia, paraneoplastic syndromes, cancers of unknown primary site and AIDS related malignancies.
